(19) United States
(12) Patent Application Publication
(10) Pub. No.: US 2003/0092757 A1
(43) Pub. Date: May 15, 2003

(54) Favorable modulation of health-related quality of life and health-related quality-adjusted time-to-progression of disease in patients with prostate cancer

(76) Inventors: Amitabh Singh, Gurnee, IL (US); Robert J. Padley, Lake Bluff, IL (US); Talat Ashraf, Vernon Hills, IL (US)

Correspondence Address: STEVEN F. WEINSTOCK; ABBOTT LABORATORIES, 100 ABBOTT PARK ROAD, DEPT. 377/AP6A, ABBOTT PARK, IL, 60064-6008, US

(21) Appl. No.: 10/118,300

(22) Filed: Apr. 8, 2002

Related U.S. Application Data
(60) Provisional application No. 60/283,159, filed on Apr. 11, 2001.

(51) Int. Cl. A61K 31/401

(52) U.S. Cl. 514/423

(57) ABSTRACT

Disclosed herein is a method for favorably modulating the health-related quality of life and the health-related quality-adjusted time-to-disease progression in a patient having prostate cancer and a method for measuring of the health-related quality-adjusted time-to-disease progression.

This application claims priority to co-pending U.S. Provisional Application Serial No. 60/283,159, filed Apr. 11, 2001, the specification of which is hereby incorporated by reference into this application.

### TECHNICAL FIELD

This invention is directed to a method for favorably modulating the health-related quality of life and the health-related quality-adjusted time-to-disease progression in a patient with prostate cancer and a method for measuring of the health-related quality-adjusted time-to-disease progression in a patient with prostate cancer.

### BACKGROUND OF THE INVENTION

Prostate cancer patients often face poor prognosis, limited treatment options, and a decline in their health-related quality of life (QoL) with disease progression. Because conventional analyses of responses in prostate cancer trials fail to account for the effect of treatment on a patient's self-perception of their health status and general well-being, qualitative and quantitative evaluation of the multidimensional health-related QoL responses of the patient over time could potentially provide a more comprehensive assessment and understanding of the benefit of a given therapeutic intervention.

Thus, there is a long-standing need in the art for a method of favorably modulating the health-related QoL and the health-related quality-adjusted time-to-progression (QATTP) of disease in patients with prostate cancer and a method for measuring the health-related QATTP of disease in patients undergoing treatment for prostate cancer.

### DISCLOSURE OF THE INVENTION

A first embodiment of this invention, therefore, is directed to a method for favorably modulating the health-related QoL of a patient with prostate cancer comprising administering thereto a therapeutically effective amount of an endothelin (ET) receptor antagonist.

A second embodiment of this invention is directed to a method for favorably modulating the health-related QATTP of disease of a patient with prostate cancer comprising administering thereto a therapeutically effective amount of an ET receptor antagonist.

As used herein, the following terms have the meanings ascribed.

The term “endothelin receptor antagonist” means a compound which binds to the endothelin receptor, which binding may be evaluated by the ability of the compound to inhibit endothelin from binding to its receptor, which inhibition is preferably between about 50%-100% at 1 μm inhibitor concentration, more preferably about 80%-100% at 1 μm inhibitor concentration, most preferably about 95%-100% at 1 μm inhibitor concentration.

The term “favorably modulating” means sustaining and/or improving the health-related QoL and/or sustaining and/or improving and/or extending the health-related QATTP of disease in a patient with prostate cancer.

The term “quality-adjusted time-to-progression of disease” or “QATTP of disease” means the interval between the initiation of chemotherapy in a patient with prostate cancer to the time of disease progression adjusted by the patient's health-related QoL score.

The term “health-related quality of life” or “health-related QoL” means domains comprising physical functioning, emotional functioning, social/family functioning, role functioning, cognitive functioning, self-perception, and other domains for patients with prostate cancer, the other domains comprising pain, fatigue, nausea and vomiting, change in appetite, dyspnea, sleep disturbance, diarrhea, constipation, urinary function, and change in weight.

A third embodiment of this invention is directed to a method for determining modulation of the health-related QATTP of disease in a patient undergoing endothelin antagonist chemotherapy for prostate cancer, the method comprising the steps of:

(a) providing a patient population,

in which the patient population comprises at least one patient with prostate cancer, preferably about 100 patients with prostate cancer, more preferably about 150 patients with prostate cancer, most preferably about 280 patients with prostate cancer;

(b) administering to each member of the patient population either a therapeutically effective amount of an ET receptor antagonist or placebo,

in which ET receptor antagonist favorably modulates, preferably sustains and/or extends, more preferably improves and/or extends, the health-related QATTP of disease of a patient with prostate cancer;

(c) measuring the health-related QoL domains of each patient over a period of time to provide a health-related QATTP of disease for each patient in the patient population,

in which the period of time is at least one interval time period between the beginning and end of the treatment, preferably about five to about seven weeks after the beginning of treatment, more preferably about six weeks after the beginning of the treatment; and

(d) determining the health-related QATTP for each health-related QoL domain and the sum of the mean or median health-related QATTP's of disease for the patient population.

A fourth embodiment of this invention is directed to a method for increasing the survival time of a patient with prostate cancer comprising administering thereto a therapeutically effective amount of an ET receptor antagonist.

In one part of the first, second, third, and fourth embodiments of this invention, the ET receptor antagonist may be administered at any time during disease progression, such as, for example, at or near beginning of disease (such as, for example, progression indicated by elevation of prostate-specific antigen levels) or toward the end of disease (such as progression indicated by signs and symptoms consistent with the progression of prostate cancer or progression indicated by hormone refractoriness).

In another part of the first, second, third, and fourth embodiments of this invention, the therapeutically effective amount of the ET receptor antagonist is between about 0.01 mg per day to about 100 mg per day, more preferably between about 1 mg per day to about 25 mg per day, most preferably about 2.5 mg or about 10 mg per day.

In still another part of the first, second, third, and fourth embodiments of this invention, the foregoing therapeutically effective amount of the ET receptor antagonist, or combinations of submultiples thereof, may be administered once or twice per day, preferably without missing a day, more preferably once per day without missing a day.

In still yet another part of the first, second, third, and fourth embodiments of this invention, the ET receptor antagonist is an endothelin A (ET_{A}) receptor antagonist, preferably an ET_{A} receptor antagonist having formula (I)-a

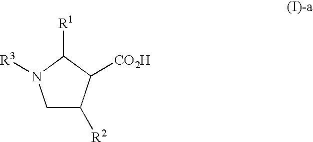

or an ET_{A} receptor antagonist having formula (I)-a with the relative or absolute stereochemistry shown in a compound having formula (I)-b

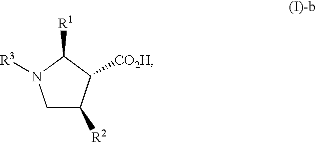

or a therapeutically acceptable salt, prodrug, or salt of prodrug of either, in which

R¹ and R² are independently alkyl, alkenyl, alkynyl, cycloalkyl, aryl, heteroaryl, or alkyl substituted with one cycloalkyl, halo, aryl, heteroaryl, heterocyclyl, —OH, or —O(alkyl) substituent;

R³ is R⁴SO₂R⁵— or R⁴C(O)R⁵—;

R⁴ is alkyl, —(CH₂)alkenyl, —(CH₂)alkynyl, —NR⁶R⁷, alkyl independently substituted with one or two cycloalkyl, aryl, heteroaryl, heterocyclyl, halo, —OH, —O(alkyl), —NH₂, —NH(alkyl), or —N(alkyl)₂ substituents, or alkenyl independently substituted with one or two cycloalkyl, aryl, heteroaryl, heterocyclyl, halo, —OH, —O(alkyl), —NH₂, —NH(alkyl), or —N(alkyl)₂ substituents;

R⁵ is a covalent bond, alkylene, —N(H)(alkylene)-, or —N(alkyl)(alkylene)-, the latter two of which are drawn from left or right, and

R⁶ and R⁷ are independently hydrogen, alkyl, —(CH₂)alkenyl, —(CH₂)alkynyl, cycloalkyl, aryl, or alkyl independently substituted with one or two cycloalkyl, aryl, heteroaryl, heterocyclyl, halo, —OH, —O(alkyl), —OCH₂CF₃, —OCH₂CF₂CF₃, —NH₂, —NH(alkyl), or —N(alkyl)₂ substituents;

in which, for the foregoing,

the term “alkenyl” means a monovalent, straight or branched hydrocarbon having two to ten carbon atoms and at least one carbon-carbon double bond, attached through a carbon atom;

the term “alkynyl” means a monovalent, straight or branched hydrocarbon, having two to ten carbon atoms and at least one carbon-carbon triple bond, attached through a carbon atom;

the term “alkyl means a monovalent, saturated, straight or branched hydrocarbon, having one to ten carbon atoms, attached through a carbon atom;

the term “aryl” means phenyl, unfused or fused with phenyl (naphthyl), cyclopentyl (indanyl), cyclopentenyl (indenyl) 1,3-dioxolanyl (1,3-benzodioxolyl), or 1,4-dioxanyl (1,4-benzodioxolyl) and unsubstituted or independently substituted with one, two, or three alkyl, halo, —CN, —OH, —CF₃, —CH₂CF₃, —CF₂CF₃, —OCF₃, —OCH₂CF₃, —OCH₂CF₂CF₃, —O(alkyl), —NO₂, —NH₂, —NH(alkyl), —N(alkyl)₂, —C(O)NH₂, —C(O)NH(alkyl), or —C(O)N(alkyl)₂ substituents;

the term “cycloalkyl” means a monovalent, saturated cyclic hydrocarbon, having three to six carbon atoms, attached through a carbon atom and unsubstituted or independently substituted with one or two alkyl, halo, —O(alkyl), ═O, —NH₂, —NH(alkyl), or —N(alkyl)₂ substituents;

the term “heteroaryl” means furanyl, oxazolyl, pyridyl, pyridazinyl, pyrimidinyl, pyrrolyl, pyrazinyl, thiazolyl, and thiophenyl, each of which is connected through a carbon atom and unsubstituted or independently substituted with one, two, or three alkyl, halo, —CN, —OH, —CF₃, —CH₂CF₃, —CF₂CF₃, —OCF₃, —OCH₂CF₃, —OCH₂CF₂CF₃, —O(alkyl), —NO₂, —NH₂, —NH(alkyl), —N(alkyl)₂, —C(O)NH₂, —C(O)NH(alkyl), or —C(O)N(alkyl)₂ substituents; and

the term “heterocyclyl” means 1,4-dioxanyl, 1,3-dioxolanyl, piperidinyl, pyrrolidinyl, morpholinyl, and thiomorpholinyl, each of which is connected through a carbon atom or nitrogen atom and unsubstituted or independently substituted with one or two alkyl, halo, —O(alkyl), ═O, —NH₂, —NH(alkyl), or —N(alkyl)₂ substituents; and

in which preferred R¹ moieties are butyl, 4-methoxyphenyl, 2,2-dimethyl-(E)-pent-3-enyl, 2,2-dimethylpentyl, 2-ethylbutyl, 3-fluoro-4-methoxyphenyl, heptyl, hexyl, 4-hydroxyphenyl, isopropyl, 2-methylbutyl, 3-methylbutyl, pentyl, propyl, 3-methyl-(E)-pent-3-enyl, 3-methylpentyl, 2-propylpentyl, and 2,2,4-trimethyl-(E)-pent-3-enyl;

preferred R² moieties are 1,3-benzodioxol-5-yl and 7-methoxy-1,3-benzodioxol-5-yl; and

preferred R³ moieties are ((N-(4-aminobutyl)-N-butylamino)carbonyl)methyl, (aminocarbonyl)methyl, ((N,N-bis(3-methylbutyl)amino)carbonyl)methyl, ((N-butylamino)carbonyl)methyl, 2-(N-butyl-N-((butyl)sulfonyl)amino)ethyl, ((N-butyl-N-(4-(dimethylamino)butyl)amino)carbonyl)methyl, ((N-butyl-N-ethylamino)carbonyl)methyl, ((N-butyl-N-methylamino)carbonyl)methyl, ((N-butyl-N-propylamino)carbonyl)methyl, 2-(N-butyl-N-((propyl)sulfonyl)amino)ethyl, 2-(N-((butyl)sulfonyl)-N-methylamino)ethyl, 2-(N-((butyl)sulfonyl)-N-(2-methylpropyl)amino)ethyl, 2-(N-((butyl)sulfonyl)-N-propylamino) ethyl, ((N-butyl-N-(3-trimethylaminopropyl)amino)carbonyl)methyl, (2-(N,N-dibutylamino)carbonyl)ethyl, ((N,N-dibutylamino)carbonyl)methyl, ((N,N-diethylamino)carbonyl)methyl, ((N,N-dihexylamino)carbonyl)methyl, ((N,N-diisobutylamino)carbonyl)methyl, ((N-(2,2-dimethylpropyl)-N-methylamino)carbonyl)methyl, ((N,N-dipentylamino)carbonyl)methyl, ((N,N-dipropylamino)carbonyl)methyl, ((1R)-1-(N,N-dipropylamino)carbonyl)but-1-yl, ((1S)-1-(N,N-dipropylamino)carbonyl)but-1-yl, 2-(N-((ethyl)sulfonyl)-N-propylamino)ethyl, 2-(N-((heptyl)sulfonyl)-N-propylamino)ethyl, ((N-hexyl-N-methylamino)carbonyl)methyl, 2-(N-((hexyl)sulfonyl)-N-propylamino)ethyl, ((N-isobutyl-N-methylamino)carbonyl)methyl, 2-(N-((isopropyl)sulfonyl)amino)ethyl, 2-(N-((3-methylbutyl)sulfonyl)-N-propylamino)ethyl, ((N-methyl-N-pentylamino)carbonyl)methyl, ((N-2-methylpropyl)carbonyl)methyl, ((N-methyl-N-propylamino)carbonyl)methyl, 2-(N-(2-methylpropyl)-N-((pentyl)sulfonyl)amino)ethyl, 2-(N-methyl-N-((propyl)sulfonyl)amino)ethyl, 2-((N-(2-methylpropyl)sulfonyl)-N-propylamino)ethyl, 2-(N-(2-methylpropyl)-N-((propyl)sulfonyl)amino)ethyl, (N-(non-5-ylamino)carbonyl)methyl, 2-(N-((pentyl)sulfonyl)-N-ethylamino)ethyl, 2-(N-((pentyl)sulfonyl)-N-propylamino)ethyl, 3-(N-((pentyl)sulfonyl)-N-propylamino)propyl, ((N-propenyl)carbonyl)methyl, ((N-propylamino)carbonyl)methyl, (2-(N-propyl-N-(((2-N,N-dimethylamino)ethyl)sulfonyl))amino)ethyl, and 2-(N-propyl-N-((propyl)sulfonyl)amino)ethyl;

which preferred variable moieties combine with the fixed moieties to form an ET_{A} receptor antagonist of the first, second, third, and fourth embodiments having formula (I)-a with the relative or absolute stereochemistry shown in the compound having formula (I)-b

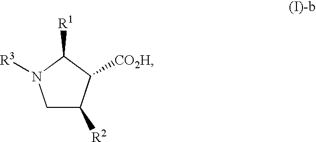

or a therapeutically acceptable salt, prodrug, or salt of a prodrug thereof, in which

R¹ is alkyl, alkenyl, or phenyl, in which the phenyl is independently substituted with one or two halo, —OH, or —O(alkyl) substituents;

R² is phenyl fused with 1,3-dioxolane and unsubstituted or substituted with one —O(alkyl) substituent;

R³ is (alkyl)SO₂N(alkyl)(alkylene)-, (alkyl)SO₂N(H)(alkylene)-, or (R⁷)(R⁸)NC(O)(alkylene)-; and

R⁷ and R⁸ are independently hydrogen, alkyl, or alkyl substituted with one —NH₂ and —N(alkyl)₂ substituent;

an ET_{A} receptor antagonist having formula (I)-a with the relative or absolute stereochemistry shown in the compound having formula (I)-b

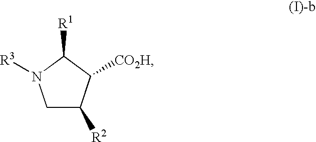

or a therapeutically acceptable salt, prodrug, or salt of a prodrug thereof, in which

R¹ is C₃-C₈-alkyl, C₆-C₈-alkenyl, or phenyl, in which the phenyl is independently substituted with one or two halo, —OH, or —O(C₁-alkyl) substituents;

R² is phenyl fused with 1,3-dioxolane and unsubstituted or substituted with one —O(C₁-alkyl) substituent;

R³ is (C₂-C₇-alkyl)SO₂N(C₁-C₄-alkyl)(C₂-C₃-alkylene)-, (C₂-C₇-alkyl)SO₂N(H)(C₁-C₂-alkylene)-, or (R⁷)(R⁸)NC(O)(C₁-C₄-alkylene)-; and

R⁷ and R⁸ are independently hydrogen, C₁-C₉-alkyl, or C₂-C₄-alkyl substituted with one —NH₂ or —N(C₁-alkyl)₂ substituent; and

an ET_{A} receptor antagonist having formula (I)-a with the relative or absolute stereochemistry shown in the compound having formula (I)-b , or a therapeutically acceptable salt, prodrug, or salt of a prodrug thereof, in which R¹ is 2,2-dimethylpentyl; R² is 1,3-benzodioxol-5-yl; and R³ is ((N,N-dibutylamino)carbonyl)methyl, (N-butyl-N-(4-(dimethylamino)butyl)amino)carbonyl)methyl, or 2-(N-((pentyl)sulfonyl)-N-propylamino)ethyl;

an ET_{A} receptor antagonist having formula (I)-a with the relative or absolute stereochemistry shown in the compound having formula (I)-b, or a therapeutically acceptable salt, prodrug, or salt of a prodrug thereof, in which R¹ is 3-fluoro-4-methoxyphenyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl,

an ET_{A} receptor antagonist having formula (I)-a with the relative or absolute stereochemistry shown in the compound having formula (I)-b, or a therapeutically acceptable salt, prodrug, or salt of a prodrug thereof, in which R¹ is 4-methoxyphenyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl;

an ET_{A} receptor antagonist having formula (I)-a with the relative or absolute stereochemistry shown in the compound having formula (I)-b, or a therapeutically acceptable salt, prodrug, or salt of a prodrug thereof, in which R¹ is 2,2-dimethylpentyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl;

an ET_{A} receptor antagonist having formula (I)-a with the relative or absolute stereochemistry shown in the compound having formula (I)-b, or a therapeutically acceptable salt, prodrug, or salt of a prodrug thereof, in which R¹ is 3-fluoro-4-methoxyphenyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl; and

an ET_{A} receptor antagonist having formula (I)-a with the relative or absolute stereochemistry shown in the compound having formula (I)-b, or a therapeutically acceptable salt, prodrug, or salt of a prodrug thereof, in which R¹ is 4-methoxyphenyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl; and

a compound, or a therapeutically acceptable salt, prodrug, or salt of a prodrug thereof, which is

trans,trans-2-(4-methoxyphenyl)-4-(1,3-benzodioxol-5-yl)-1-(((N-propylamino)carbonyl)methyl)pyrrolidine-3-carboxylic acid,

trans,trans-2-(4-methoxyphenyl)-4-(1,3-benzodioxol-5-yl)-1-(aminocarbonyl)methyl)pyrrolidine-3-carboxylic acid,

trans,trans-2-(4-methoxyphenyl)-4-(1,3-benzodioxol-5-yl)-1-(((N-propenyl)carbonyl)methyl)pyrrolidine-3-carboxylic acid,

trans,trans-2-(4-methoxyphenyl)-4-(1,3-benzodioxol-5-yl)-1-(((N-butylamino)carbonyl)methyl)pyrrolidine-3-carboxylic acid,

trans,trans-2-(4-methoxyphenyl)-4-(1,3-benzodioxol-5-yl)-1-(((N-methyl-N-propylamino)carbonyl)methyl)pyrrolidine-3-carboxylic acid,

trans,trans-2-(4-methoxyphenyl)-4-(1,3-benzodioxol-5-yl)-1-(((N-2-methylpropyl)carbonyl)methyl)pyrrolidine-3-carboxylic acid,

trans,trans-2-(4-methoxyphenyl)-4-(1,4-benzodioxol-6-yl)-1-(((N-propylamino)carbonyl)methyl)pyrrolidine-3-carboxylic acid,

trans,trans-2-(4-methoxyphenyl)-4-(1,4-benzodioxol-6-yl)-1-(((N-methyl-N-propylamino)carbonyl)methyl)pyrrolidine-3-carboxylic acid,

trans,trans-2-(4-methoxyphenyl)-4-(1,3-benzodioxol-5-yl)-1-(((N-butyl-N-methylamino)carbonyl)methyl)pyrrolidine-3-carboxylic acid,

trans,trans-2-(4-methoxyphenyl)-4-(1,3-benzodioxol-5-yl)-1-(((N,N-bis(3-methylbutyl)amino)carbonyl)methyl)pyrrolidine-3-carboxylic acid,

trans,trans-2-(4-methoxyphenyl)-4-(1,3-benzodioxol-5-yl)-1-(((N,N-dipentylamino)carbonyl)methyl)pyrrolidine-3-carboxylic acid,

trans,trans-2-(4-methoxyphenyl)-4-(1,3-benzodioxol-5-yl)-1-(((N-methyl-N-pentylamino)carbonyl)methyl)pyrrolidine-3-carboxylic acid,

trans,trans-2-(4-methoxyphenyl)-4-(1,3-benzodioxol-5-yl)-1-(((N,N-diisobutylamino)carbonyl)methyl)pyrrolidine-3-carboxylic acid,

trans,trans-2-(4-methoxyphenyl)-4-(1,3-benzodioxol-5-yl)-1-(((N-hexyl-N-methylamino)carbonyl)methyl)pyrrolidine-3-carboxylic acid,

trans,trans-2-(4-methoxyphenyl)-4-(1,3-benzodioxol-5-yl)-1-(((N,N-dibutylamino)carbonyl)methyl)pyrrolidine-3-carboxylic acid,

trans,trans-2-(4-methoxyphenyl)-4-(1,3-benzodioxol-5-yl)-1-(((N,N-diethylamino)carbonyl)methyl)pyrrolidine-3-carboxylic acid,

trans,trans-2-(4-methoxyphenyl)-4-(1,3-benzodioxol-5-yl)-1-(((N,N-dipropylamino)carbonyl)methyl)pyrrolidine-3-carboxylic acid,

trans,trans-2-(4-methoxyphenyl)-4-(1,3-benzodioxol-5-yl)-1-(((N-isobutyl-N-methylamino)carbonyl)methyl)pyrrolidine-3-carboxylic acid,

trans,trans-2-(4-methoxyphenyl)-4-(1,3-benzodioxol-5-yl)-1-(2-(N-((isopropyl)sulfonyl)amino)ethyl)pyrrolidine-3-carboxylic acid,

trans,trans-2-(4-methoxyphenyl)-4-(1,3-benzodioxol-5-yl)-1-(((N-butyl-N-ethylamino)carbonyl)methyl)pyrrolidine-3-carboxylic acid,

trans,trans-2-(4-methoxyphenyl)-4-(1,3-benzodioxol-5-yl)-1-(((N-(2,2-dimethylpropyl)-N-methylamino)carbonyl)methyl)pyrrolidine-3-carboxylic acid,

trans,trans-2-(4-methoxyphenyl)-4-(1,3-benzodioxol-5-yl)-1-(2-(N-((butyl)sulfonyl)-N-methylamino)ethyl)pyrrolidine-3-carboxylic acid,

trans,trans-2-(4-methoxyphenyl)-4-(1,3-benzodioxol-5-yl)-1-(2-(N-methyl-N-((propyl)sulfonyl)amino)ethyl)pyrrolidine-3-carboxylic acid,

(2R,3R,4R)-2-(4-methoxyphenyl)-4-(1,3-benzodioxol-5-yl)-1-(((1R)-1-(N,N-dipropylamino)carbonyl)but-1-yl)pyrrolidine-3-carboxylic acid,

(2S,3S,4S)-2-(4-methoxyphenyl)-4-(1,3-benzodioxol-5-yl)-1-(((1R)-1-(N,N-dipropylamino)carbonyl)but-1-yl)pyrrolidine-3-carboxylic acid,

(2S,3S,4S)-2-(4-methoxyphenyl)-4-(1,3-benzodioxol-5-yl)-1-(((1S)-1-(N,N-dipropylamino)carbonyl)but-1-yl)pyrrolidine-3-carboxylic acid,

(2R,3R,4R)-2-(4-methoxyphenyl)-4-(1,3-benzodioxol-5-yl)-1-(((1S)-1-(N,N-dipropylamino)carbonyl)but-1-yl)pyrrolidine-3-carboxylic acid,

trans,trans-2-(4-methoxyphenyl)-4-(1,3-benzodioxol-5-yl)-1-(((N-butyl-N-propylamino)carbonyl)methyl)pyrrolidine-3-carboxylic acid,

trans,trans-2-(4-methoxyphenyl)-4-(1,3-benzodioxol-5-yl)-1-(2-(N,N-dibutylamino)carbonyl)ethyl)pyrrolidine-3-carboxylic acid,

trans,trans-2-(4-methoxyphenyl)-4-(1,3-benzodioxol-5-yl)-1-(((R,S)-2-(N,N-dibutylamino)carbonyl)ethyl)pyrrolidine-3-carboxylic acid,

trans,trans-2-pentyl-4-(1,3-benzodioxol-5-yl)-1-((N,N-dibutylamino)carbonyl)methyl)pyrrolidine-3-carboxylic acid,

trans,trans-2-pentyl-4-(1,3-benzodioxol-5-yl)-1-(2-(N-propyl-N-((propyl)sulfonyl)amino)ethyl)pyrrolidine-3-carboxylic acid,

trans,trans-2-propyl-4-(1,3-benzodioxol-5-yl)-1-(((N,N-dibutylamino)carbonyl)methyl)pyrrolidine-3-carboxylic acid,

(2R,3R,4S)-2-(4-methoxyphenyl)-4-(1,3-benzodioxol-5-yl)-1-(((N,N-dibutylamino)carbonyl)methyl)pyrrolidine-3-carboxylic acid,

trans,trans-2-(4-methoxyphenyl)-4-(1,3-benzodioxol-5-yl)-1-(2-(N-butyl-N-((propyl)sulfonyl)amino)ethyl)pyrrolidine-3-carboxylic acid,

trans,trans-2-(4-methoxyphenyl)-4-(1,3-benzodioxol-5-yl)-1-(((N,N-dibutylamino)carbonyl)methyl)pyrrolidine-3-carboxylic acid,

trans,trans-2-(4-methoxyphenyl)-4-(1,3-benzodioxol-5-yl)-1-(2-(N-propyl-N-((propyl)sulfonyl)amino)ethyl)pyrrolidine-3-carboxylic acid,

trans,trans-2-(4-methoxyphenyl)-4-(1,3-benzodioxol-5-yl)-1-(2-(N-butyl-N-((butyl)sulfonyl)amino)ethyl)pyrrolidine-3-carboxylic acid,

trans,trans-2-(4-hydroxyphenyl)-4-(1,3-benzodioxol-5-yl)-1-(((N,N-dibutylamino)carbonyl)methyl)pyrrolidine-3-carboxylic acid,

trans,trans-2-(4-methoxyphenyl)-4-(1,3-benzodioxol-5-yl)-1-(2-(N-(2-methylpropyl)-N-((propyl)sulfonyl)amino)ethyl)pyrrolidine-3-carboxylic acid,

trans,trans-2-(4-methoxyphenyl)-4-(1,3-benzodioxol-5-yl)-1-(2-(N-((ethyl)sulfonyl)-N-propylamino)ethyl)pyrrolidine-3-carboxylic acid,

trans,trans-2-(4-methoxyphenyl)-4-(1,3-benzodioxol-5-yl)-1-(2-(N-((butyl)sulfonyl)-N-propylamino)ethyl)pyrrolidine-3-carboxylic acid,

trans,trans-2-(isopropyl)-4-(1,3-benzodioxol-5-yl)-1-(((N,N-dibutylamino)carbonyl)methyl)pyrrolidine-3-carboxylic acid,

trans,trans-2-(4-methoxyphenyl)-4-(1,3-benzodioxol-5-yl)-1-(2-N-((pentyl)sulfonyl)-N-propylamino)ethyl)pyrrolidine-3-carboxylic acid,

trans,trans-2-(4-methoxyphenyl)-4-(1,3-benzodioxol-5-yl)-1-(2-((N-(2-methylpropyl)sulfonyl)-N-propylamino)ethyl)pyrrolidine-3-carboxylic acid,

trans,trans-2-(4-methoxyphenyl)-4-(1,3-benzodioxol-5-yl)-1-(2-(N-((heptyl)sulfonyl)-N-propylamino)ethyl)pyrrolidine-3-carboxylic acid,

trans,trans-2-(4-methoxyphenyl)-4-(1,3-benzodioxol-5-yl)-1-(2-(N-((hexyl)sulfonyl)-N-propylamino)ethyl)pyrrolidine-3-carboxylic acid,

trans,trans-2-(4-methoxyphenyl)-4-(1,3-benzodioxol-5-yl)-1-(2-(N-((pentyl)sulfonyl)-N-ethylamino)ethyl)pyrrolidine-3-carboxylic acid,

trans,trans-2-(3-fluoro-4-methoxyphenyl)-4-(1,3-benzodioxol-5-yl)-1-(((N,N-dibutylamino)carbonyl)methyl)pyrrolidine-3-carboxylic acid,

trans,trans-2-propyl-4-(1,3-benzodioxol-5-yl)-1-(2-(N-((pentyl)sulfonyl)-N-propylamino)ethyl)pyrrolidine-3-carboxylic acid,

trans,trans-2-(3-fluoro-4-methoxyphenyl)-4-(1,3-benzodioxol-5-yl)-1-(2-(N-((pentyl)sulfonyl)-N-propylamino)ethyl)pyrrolidine-3-carboxylic acid,

trans,trans-2-(3-fluoro-4-methoxyphenyl)-4-(1,3-benzodioxol-5-yl)-1-(2-(N-((butyl)sulfonyl)-N-propylamino)ethyl)pyrrolidine-3-carboxylic acid,

trans,trans-2-(4-methoxyphenyl)-4-(1,3-benzodioxol-5-yl)-1-(3-(N-((pentyl)sulfonyl)-N-propylamino)propyl)pyrrolidine-3-carboxylic acid,

trans,trans-2-butyl-4-(1,3-benzodioxol-5-yl)-1-(((N,N-dibutylamino)carbonyl)methyl)pyrrolidine-3-carboxylic acid,

trans,trans-2-(2-methylbutyl)-4-(1,3-benzodioxol-5-yl)-1-(((N,N-dibutylamino)carbonyl)methyl)pyrrolidine-3-carboxylic acid,

trans,trans-2-(3-methylbutyl)-4-(1,3-benzodioxol-5-yl)-1-(((N,N-dibutylamino)carbonyl)methyl)pyrrolidine-3-carboxylic acid,

trans,trans-2-(3-fluoro-4-methoxyphenyl)-4-(1,3-benzodioxol-5-yl)-1-(2-(N-((hexyl)sulfonyl)-N-propylamino)ethyl)pyrrolidine-3-carboxylic acid,

trans,trans-2-hexyl-4-(1,3-benzodioxol-5-yl)-1-(((N,N-dibutylamino)carbonyl)methyl)pyrrolidine-3-carboxylic acid,

trans,trans-2-(3-fluoro-4-methoxyphenyl)-4-(7-methoxy-1,3-benzodioxol-5-yl)-1-(((N,N-dibutylamino)carbonyl)methyl)pyrrolidine-3-carboxylic acid,

trans,trans-2-(3-fluoro-4-methoxyphenyl)-4-(7-methoxy-1,3-benzodioxol-5-yl)-1-(((N-butyl-N-propylamino)carbonyl)methyl)pyrrolidine-3-carboxylic acid,

trans,trans-2-heptyl-4-(1,3-benzodioxol-5-yl)-1-(((N,N-dibutylamino)carbonyl)methyl)pyrrolidine-3-carboxylic acid,

trans,trans-2-(3-fluoro-4-methoxyphenyl)-4-(1,3-benzodioxol-5-yl)-1-(2-(N-((3-methylbutyl)sulfonyl)-N-propylamino)ethyl)pyrrolidine-3-carboxylic acid,

trans,trans-2-(3-fluoro-4-methoxyphenyl)-4-(7-methoxy-1,3-benzodioxol-5-yl)-1-(2-(N-((pentyl)sulfonyl)-N-propylamino)ethyl)pyrrolidine-3-carboxylic acid,

trans,trans-2-(3-fluoro-4-methoxyphenyl)-4-(1,3-benzodioxol-5-yl)-1-(2-(N-(2-methylpropyl)-N-((pentyl)sulfonyl)amino)ethyl)pyrrolidine-3-carboxylic acid,

trans,trans-2-(4-methoxyphenyl)-4-(1,3-benzodioxol-5-yl)-1-((N-(non-5-ylamino)carbonyl)methyl)pyrrolidine-3-carboxylic acid,

trans,trans-2-(3-fluoro-4-methoxyphenyl)-4-(1,3-benzodioxol-5-yl)-1-(2-(N-((2-methylpropyl)sulfonyl)-N-propylamino)ethyl)pyrrolidine-3-carboxylic acid,

trans,trans-2-(4-methoxyphenyl)-4-(1,3-benzodioxol-5-yl)-1-(((N,N-dihexylamino)carbonyl)methyl)pyrrolidine-3-carboxylic acid,

trans,trans-2-(4-methoxyphenyl)-4-(1,3-benzodioxol-5-yl)-1-(((N-butyl-N-(hept-4-yl)amino)carbonyl)methyl)pyrrolidine-3-carboxylic acid,

trans,trans-2-(2-propylpentyl)-4-(1,3-benzodioxol-5-yl)-1-(((N,N-dibutylamino)carbonyl)methyl)pyrrolidine-3-carboxylic acid,

trans,trans-2-(3-methylpentyl)-4-(1,3-benzodioxol-5-yl)-1-(((N,N-dibutylamino)carbonyl)methyl)pyrrolidine-3-carboxylic acid,

trans,trans-2-(2-ethylbutyl)-4-(1,3-benzodioxol-5-yl)-1-(((N,N-dibutylamino)carbonyl)methyl)pyrrolidine-3-carboxylic acid,

trans,trans-2-(3-fluoro-4-methoxyphenyl)-4-(1,3-benzodioxol-5-yl)-1-(2-(N-((butyl)sulfonyl)-N-(2-methylpropyl)amino)ethyl)pyrrolidine-3-carboxylic acid,

trans,trans-2-(3-methyl-(E)-pent-3-en-1-yl)-4-(1,3-benzodioxol-5-yl)-1-(((N,N-dibutylamino)carbonyl)methyl)pyrrolidine-3-carboxylic acid,

trans,trans-2-(2-methylpentyl)-4-(1,3-benzodioxol-5-yl)-1-(((N,N-dibutylamino)carbonyl)methyl)pyrrolidine-3-carboxylic acid,

trans,trans-2-(2,2-dimethylphenyl)-4-(1,3-benzodioxol-5-yl)-1-(((N,N-dibutylamino)carbonyl)methyl)pyrrolidine-3-carboxylic acid,

trans,trans-2-(2,2,4-trimethyl-3-(E)-pent-3-enyl)-4-(1,3-benzodioxol-5-yl)-1-(((N,N-dibutylamino)carbonyl)methyl)pyrrolidine-3-carboxylic acid,

trans,trans-2-(2,2,-dimethylpentyl)-4-(7-methoxy-1,3-benzodioxol-5-yl)-1-(((N,N-dibutylamino)carbonyl)methyl)pyrrolidine-3-carboxylic acid,

trans,trans-2-(2,2-dimethyl-(E)-pent-3-enyl)-4-(7-methoxy-1,3-benzodioxol-5-yl)-1-(((N,N-dibutylamino)carbonyl)methyl)pyrrolidine-3-carboxylic acid,

trans,trans-2-(4-methoxyphenyl)-4-(1,3-benzodioxol-5-yl)-1-(((N-(4-aminobutyl)-N-butylamino)carbonyl)methyl)pyrrolidine-3-carboxylic acid,

trans,trans-2-(4-methoxyphenyl)-4-(1,3-benzodioxol-5-yl)-1-(((N-butyl-N-(4-(dimethylamino)butyl)amino)carbonyl)methyl)pyrrolidine-3-carboxylic acid,

(2R,3R,4S)-2-(3-fluoro-4-methoxyphenyl)-4-(1,3-benzodioxol-5-yl)-1-(2-(N-((pentyl)sulfonyl)-N-propylamino)ethyl)pyrrolidine-3-carboxylic acid,

(2S,3R,4S)-2-(2,2-dimethylpentyl)-4-(7-methoxy-1,3-benzodioxol-5-yl)-1-(((N,N-dibutylamino)carbonyl)methyl)pyrrolidine-3-carboxylic acid,

(2S,3R,4S)-2-(2,2-dimethylpentyl)-4-(1,3-benzodioxol-5-yl)-1-(((N,N-dibutylamino)carbonyl)methyl)pyrrolidine-3-carboxylic acid,

(2S,3R,4S)-2-(4-methoxyphenyl)-4-(1,3-benzodioxol-5-yl)-1-(((N-butyl-N-(4-(dimethylamino)butyl)amino)carbonyl)methyl)pyrrolidine-3-carboxylic acid,

(2S,3R,4S)-2-(2,2-dimethyl-(E)-pent-3-enyl)-4-(1,3-benzodioxol-5-yl)-1-(((N,N-dibutylamino)carbonyl)methyl)pyrrolidine-3-carboxylic acid, and

(2S,3R,4S)-2-(2,2-dimethyl-(E)-pent-3-enyl)-4-(7-methoxy-1,3-benzodioxol-5-yl)-1-(((N,N-dibutylamino)carbonyl)methyl)pyrrolidine-3-carboxylic acid;

preferred compounds of which are

trans,trans-2-(4-methoxyphenyl)-4-(1,3-benzodioxol-5-yl)-1-(((N,N-dibutylamino)carbonyl)methyl)pyrrolidine-3-carboxylic acid,

trans,trans-2-(3-fluoro-4-methoxyphenyl)-4-(1,3-benzodioxol-5-yl)-1-(2-(N-((pentyl)sulfonyl)-N-propylamino)ethyl)pyrrolidine-3-carboxylic acid,

trans,trans-2-(2,2-dimethylpentyl)-4-(7-methoxy-1,3-benzodioxol-5-yl)-1-(((N,N-dibutylamino)carbonyl)methyl)pyrrolidine-3-carboxylic acid,

trans,trans-2-(2,2-dimethylpentyl)-4-(1,3-benzodioxol-5-yl)-1-(((N,N-dibutylamino)carbonyl)methyl)pyrrolidine-3-carboxylic acid,

trans,trans-2-(4-methoxyphenyl)-4-(1,3-benzodioxol-5-yl)-1-(((N-butyl-N-(4-(dimethylamino)butyl)amino)carbonyl)methyl)pyrrolidine-3-carboxylic acid, and

trans,trans-2-(2,2-dimethyl-(E)-pent-3-enyl)-4-(7-methoxy-1,3-benzodioxol-5-yl)-1-(((N,N-dibutylamino)carbonyl)methyl)pyrrolidine-3-carboxylic acid;

more preferred compounds of which include

(2R,3R,4S)-2-(4-methoxyphenyl)-4-(1,3-benzodioxol-5-yl)-1-(((N,N-dibutylamino)carbonyl)methyl)pyrrolidine-3-carboxylic acid,

(2R,3R,4S)-2-(3-fluoro-4-methoxyphenyl)-4-(1,3-benzodioxol-5-yl)-1-(2-(N-((pentyl)sulfonyl)-N-propylamino)ethyl)pyrrolidine-3-carboxylic acid,

(2S,3R,4S)-2-(2,2-dimethylpentyl)-4-(7-methoxy-1,3-benzodioxol-5-yl)-1-(((N,N-dibutylamino)carbonyl)methyl)pyrrolidine-3-carboxylic acid,

(2S,3R,4S)-2-(2,2-dimethylpentyl)-4-(1,3-benzodioxol-5-yl)-1-(((N,N-dibutylamino)carbonyl)methyl)pyrrolidine-3-carboxylic acid,

(2S,3R,4S)-2-(4-methoxyphenyl)-4-(1,3-benzodioxol-5-yl)-1-(((N-butyl-N-(4-(dimethylamino)butyl)amino)carbonyl)methyl)pyrrolidine-3-carboxylic acid,

(2S,3R,4S)-2-(2,2-dimethyl-(E)-pent-3-enyl)-4-(1,3-benzodioxol-5-yl)-1-(((N,N-dibutylamino)carbonyl)methyl)pyrrolidine-3-carboxylic acid, and

(2S,3R,4S)-2-(2,2-dimethyl-(E)-pent-3-enyl)-4-(7-methoxy-1,3-benzodioxol-5-yl)-1-(((N,N-dibutylamino)carbonyl)methyl)pyrrolidine-3-carboxylic acid; and

a most preferred compound of which is

(2R,3R,4S)-2-(4-methoxyphenyl)-4-(1,3-benzodioxol-5-yl)-1-(((N,N-dibutylamino)carbonyl)methyl)pyrrolidine-3-carboxylic acid, also known as atrasentan.

The ET receptor antagonists of this invention comprise asymmetrically substituted carbon atoms in the R or S configuration, in which the terms “R” and “S” are as defined by the IUPAC 1974 Recommendations for Section E, Fundamental Stereochemistry, Pure Appl. Chem. (1976) 45, 13-10. ET receptor antagonists having asymmetrically substituted carbon atoms with equal amounts of R and S configurations are racemic at those carbon atoms. Atoms with an excess of one configuration over the other are assigned the configuration in the higher amount, preferably an excess of about 85%-90%, more preferably an excess of about 95%-99%, and still more preferably an excess greater than about 99%. Accordingly, this invention is meant to embrace racemic mixtures, relative and absolute stereoisomers, and mixtures of relative and absolute stereoisomers of the ET receptor antagonists therein.

The term “relative stereochemistry,” as used herein, refers to the direction of the variable R¹ and R² moieties in relation to the direction of the fixed carboxyl moiety to which each is adjacent. In a preferred embodiment, R¹ and R² are in the opposite direction of the carboxyl moiety and form the “trans,trans-” stereochemistry shown in the compound having formula (I)-b.

The term “absolute stereochemistry,” as used herein, refers to the fixed direction of each fixed or variable moiety regardless of the orientation of the other substituents.

The compounds having formula (I)-a and formula (I)-b may also contain carbon-carbon double bonds in the Z or E configuration, in which the term “Z” represents the larger two of the four substituents on same side of a carbon-carbon double bond and the term “E” represents the larger two of the four substituents on opposite sides of a carbon-carbon double bond. The compounds having formula (I)-a and formula (I)-b may also exist as an equilibrium mixture of Z and E configurations.

Compounds having formula (I)-a and formula (I)-b containing hydroxyl, amino, or carboxylic acids may have attached thereto prodrug-forming moieties. The prodrug-forming moieties are removed by metabolic processes and release the compounds having the freed hydroxyl, amino, or carboxylic acid in vivo. Prodrugs are useful for adjusting such pharmacokinetic properties of the compounds, or their metabolites, as solubility and/or hydrophobicity, absorption in the gastrointestinal tract, bioavailability, tissue penetration, and rate of clearance. Examples of prodrugs of the compounds include ones in which the carboxyl moiety of the compounds have attached thereto a methyl, ethyl, isopropyl, or tert-butyl moiety.

The compounds having formula (I)-a and formula (I)-b may be prepared by synthetic processes or metabolic processes. Metabolic processes include those processes occurring in vitro or in vivo. An example of a metabolite of the compounds is one in which R¹ is 4-methoxyphenyl; R² is 1,3-benzodioxol-5-yl; and R³ is ((N-butylamino)carbonyl)methyl.

The compounds having formula (I)-a and formula (I)-b may exist as acid addition salts, basic addition salts, or zwitterions. Salts of the compounds are prepared during their isolation or following their purification. Acid addition salts of the compounds are those derived from the reaction of the same with an acid. For example, the acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, formate, fumarate, hydrochloride, hydrobromide, hydroiodide, lactate, maleate, mesitylenesulfonate, methanesulfonate, naphthylenesulfonate, nicotinate, oxalate, pectinate, persulfate, picrate, propionate, succinate, tartrate, thiocyanate, trichloroacetic, trifluoroacetic, phosphate, glutamate, bicarbonate, para-toluenesulfonate, lactobionate, and undecanoate salts of the compounds and prodrugs thereof are contemplated as being within the scope of this invention. Because the compounds contain carboxylic acids, basic addition salts may be prepared therefrom by reaction with a base such as the hydroxide, carbonate, or bicarbonate of cations such as lithium, sodium, potassium, calcium, and magnesium. A preferred salt of the compounds is the hydrochloride salt.

The compounds having formula (I)-a and formula (I)-b may be administered with or without an excipient and with or without another chemotherapeutic agent. Excipients include encapsulating materials or formulation additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, and mixtures thereof. Excipients for orally administered compounds in solid dosage forms include agar, alginic acid, cocoa butter, gelatin, isotonic saline, malt, powdered tragacanth, Ringer's solution, talc, water, aluminum hydroxide, magnesium hydroxide, sodium and potassium phosphate salts, cellulose, cellulose acetate, ethyl cellulose, sodium carboxymethyl cellulose, ethyl laureate, ethyl oleate, magnesium stearate, sodium lauryl sulfate, castor oil, corn oil, cottonseed oil, germ oil, groundnut oil, olive oil, peanut oil, safflower oil, sesame oil, soybean oil, benzyl alcohol, benzyl benzoate, 1,3-butylene glycol, ethanol, ethyl acetate, ethyl carbonate, glycerol, isopropanol, propylene glycol, tetrahydrofurfuryl alcohol, corn starch, potato starch, lactose, glucose sucrose, and mixtures thereof. Excipients for ophthalmically and orally administered compounds in liquid dosage forms include water, ethanol, isopropanol, ethyl carbonate, ethyl acetate, benzyl alcohol, benzyl benzoate, propylene glycol, 1,3-butylene glycol, cottonseed oil, groundnut oil, corn oil, germ oil, olive oil, castor oil, sesame oil, glycerol, tetrahydrofurfuryl alcohol, polyethylene glycols, fatty acid esters of sorbitan, and mixtures thereof. Excipients for osmotically administered compounds include water, ethanol, isopropanol, chlorofluorohydrocarbons, and mixtures thereof. Excipients for parenterally administered compounds include water, 1,3-butanediol, Ringer's solution, U.S.P. or isotonic sodium chloride solution, oleic acid, castor oil, corn oil, cottonseed oil, germ oil, groundnut oil, olive oil, peanut oil, safflower oil, sesame oil, soybean oil, liposomes, and mixtures thereof. Excipients for rectally administered compounds include cocoa butter, polyethylene glycol, wax, and mixtures thereof.

The compounds having formula (I)-a and formula (I)-b may be administered as the sole active agent, or they may also be used co-therapeutically with one or more anticancer drugs or methods including hormonal agents such as leuprolide (Lupron®); gonadorelin antagonists such as goserelin (Zoladex®) and abarelix; bicalutamide; nilutamide; flutamide; vitamin D; vitamin D analogues; estrogen and estrogen analogues such as diethylstibestrol; prednisone; hydrocortisone; ketoconazole; cyproterone acetate; progesterone; 5-alpha reductase inhibitors such as finasteride; bone-seeking radionuclides such as samarium (Quadramet®), strontium (Metastron®), and ¹⁸⁶rhenium; external beam radiation such as three dimensional conformal radiation; brachytherapy (the implantation of radioactive seeds in the prostate); monoclonal antibodies such as trastuzumab (Herceptin®); anti-angiogenic drugs such as thrombospondin peptide or kringle 5; matrix metalloproteinase inhibitors; farnesyl transferase inhibitors; lycopenes; urokinase; plasminogen activator inhibitors; plasminogen activator receptor blockers; apoptosis inducers; selective and non-selective alpha blockers; platinum agents such as cis-platinum and carbo-platinum; taxane- class drugs such as docetaxil and paclitaxil; estramustine; gemcytabine; adriamycin; doxorubicin; daunorubicin; mitoxantrone; vinblastine; vincristine; capecitabine; irinotecan; topotecan; 5-fluorouracil; interferons; cytoxan; methotrexate; cytokines such as IL-2; PPAR agonists such as thiazolidine diones; retinoid-type agents; 5-lipooxygenase inhibitors such as zyflo (Zilueton®); COX-2 inhibitors; gene-therapy based therapeutics, including sense and anti-sense polynucleotides; cholesterol lowering drugs such as lovastatin, pravastatin, and simvistatin; bisphosphonates such as etidronate, ibandronate, pamidronate, and risendronate; osteoprotegrin; antibodies, both monoclonal and polyclonal; antibody-coupled radionucleotides; antibody-coupled cytotoxic agents; antibody-coupled radionucleotides; viral-vector delivered agents; vaccines directed at protein, carbohydrate, or nucleic acid targets; aminoglutethimide; and suramin.

These combinations may be administered separately or singly in dosage forms containing both or all drugs. When administered as a combination, the drugs may be formulated as separate compositions, given at the same time or different times, or the therapeutic agents may be given as a single composition.

The compounds having formula (I)-a and formula (I)-b may be administered parenterally (subcutaneously, intravenously, intramuscularly, and intrastemally), orally, osmotically, ophthalmically, rectally, topically, and transdermally. Orally administered compounds in solid dosage forms may be administered as capsules, dragees, granules, pills, powders, and tablets. Ophthalmically and orally administered compounds in liquid dosage forms may be administered as elixirs, emulsions, microemulsions, solutions, suspensions, and syrups. Osmotically and topically administered compounds may be administered as creams, gels, inhalants, lotions, ointments, pastes, powders, solutions, and sprays. Parenterally administered compounds may be administered as aqueous or oleaginous solutions or aqueous or oleaginous suspensions, the latter of which contains crystalline, amorphous, or otherwise insoluble forms of the compounds. Rectally administered compounds may be administered as creams, gels, lotions, ointments, and pastes. A preferred means of administration of the compounds is orally.

The preparation of the compounds having formula (I)-a and formula (I)-b and their binding affinity for ET receptors are disclosed in commonly-owned, U.S. Pat. Nos. 5,731,434, 5,622,971, and 5,767,144 and commonly owned published PCT applications WO/06095, published Feb. 29, 1996; WO 97/30045, published Aug. 21, 1997; and WO 99/06397, published Feb. 11, 1999.

### Determination of Health-Related Quality-Adjusted Time-to-Disease Progression

The health-related QATTP of disease model for this invention expresses progression-free time as an equally preferable amount of time spent in full health. This is achieved by using patient-reported health-related QoL, as measured for the duration of observation or progression-free interval, to weight progression-free time. These data were collected from randomized patients having hormone refractory prostate cancer (HRPCa) with the following validated instruments: the European Organization for Research and Treatment of Cancer Quality of Life Questionnaire (EORTC QLQ-30) and the Functional Assessment of Cancer Therapy (FACT-G) and its prostate cancer-specific module (FACT-P).

Patients received treatment with 10 mg (N=89) or 2.5 mg (N=95) of atrasentan or placebo (N=104) until experiencing a clinical event indicative of disease progression such as palliative opiate treatment of new bone or visceral pain, palliative radiation treatment of new bone pain, or new tumor growth symptoms requiring intervention or treatment with chemotherapy.

Patient-reported health-related QoL data were collected with the EORTC QLQ-30 and the FACT-G and FACT-P, both of which were administered at baseline, at six week intervals and at each patient's final visit. The results from the 10 mg and 2.5 mg treatment groups are reported relative to the placebo group.

A patient's transformed domain score and total score from both the EORTC QLQ-30 and FACT were used to weight the time-to-progression outcome data. Transformed domain scores ranged between 0 and 1, so the reported health-related QATTP of disease outcome was never larger than the actual time-to-progression. The methods used for converting domain scores to weight adjustments are shown in TABLE 1.

TABLE 1
TRANSFORMATION OF HEALTH-RELATED QUALITY OF LIFE INSTRUMENT
DOMAIN SCORES TO WEIGHTED ADJUSTMENTS

| | DOMAIN SCORE | CONVERSION METHOD |
|---|---|---|
| INSTRUMENT AND DOMAIN NAME | RANGE | TO UNIT SCALE |
| EORTC Physical Functioning, | 0-100 | Domain Score/100 |
| Emotional Functioning, Role | | |
| Functioning, Social Functioning, | 0-100 | |
| Cognitive Functioning, and Global | | |
| Score^{a} | | |
| EORTC Pain, Fatigue, Nausea and | 0-100 | 1 − (Domain Score/100) |
| Vomiting, Appetite Loss, Dyspnea, | | |
| Sleep Disturbance, Diarrhea, | | |
| Constipation^{b} | | |
| FACT Physical, Social/Family. Functional Well-being^{a} | 0-28 | $\frac{\begin{matrix}Domain Score - \\ Lowest Domain Score\end{matrix}}{Domain Score Range}$ |
| FACT Emotional Well-being^{a} | 0-20 | $\frac{\begin{matrix}Domain Score - \\ Lowest Domain Score\end{matrix}}{Domain Score Range}$ |
| FACT-G^{a} | 0-112 | $\frac{\begin{matrix}Domain Score - \\ Lowest Domain Score\end{matrix}}{Domain Score Range}$ |
| FACT-P^{a} | 0-48 | $\frac{\begin{matrix}Domain Score - \\ Lowest Domain Score\end{matrix}}{Domain Score Range}$ |
| FACT-Total^{a} | 0-160 | $\frac{\begin{matrix}Domain Score - \\ Lowest Domain Score\end{matrix}}{Domain Score Range}$ |

^{a} A higher score means a better health-related QoL. A higher transformed score means improved health-related QoL. ^{b} A higher score means a worse health-related QoL. A higher transformed score means improved symptoms.

Possible scores for the fourteen EORTC domains each range between 1 and 100. For six domains (physical, emotional, role, social, and cognitive functioning and global score), a higher score means a better health-related QoL. These six scores were transformed to weights by dividing the patient-reported scores by 100.

For the remaining eight EORTC domains, a higher score indicates worse symptoms (a worse health-related QoL). These domains are pain, fatigue, nausea and vomiting, appetite loss, dysnepa, sleep disturbance, diarrhea, and constipation. These eight scores were converted to weight adjustments by dividing them by 100 and subtracting the result from the integer 1 to provide consistent directionality of response. FACT domain scores were converted to weights using the linear affine transformation suggested in SF-36 Health Survey Manual and Interpretation Guide.

Each patient's health-related QATTP of disease was computed as the sum of the health-related QoL weights multiplied by the duration for which that patient experienced that health-related QoL.

If a patient experienced a clinical event between two health-related QoL assessments, the set of health-related QoL domain scores immediately prior to the event were carried forward to the time of the clinical event. The mean and median health-related QATTP of disease outcomes were then estimated using Kaplan-Meier product limit methodology (Journal of the American Statistical Association, vol. 53, 1958, pp 457-481). The area under a Kaplan-Meier survival curve conveys an estimated mean health-related QATTP of disease. This analysis was applied to both intent-to-treat and per protocol population data. All health-related QATTP of disease comparisons between atrasentan and placebo treatment groups were based on a log-rank test with statistical significance at an α of 0.05.

Results of the Kaplan-Meier product limit survival method analysis are reported in TABLE 2 (Intent to Treat) and TABLE 3 (Per Protocol Population). Mean and median health-related QATTP of disease are shown by treatment group. Log-rank tests comparing the differences between treatment groups are also reported.

The Kaplan-Meier product limit method may provide biased results if study data are obtained under certain conditions such as staggered entry of subjects into the study and/or incomplete follow-up (Biometrics. 1989; 5:781-795). Thus, a second analysis was implemented to verify that the conclusions derived from the Kaplan-Meier method would remain robust to the length of follow-up. The assumption was that all patients were followed for one year. Patients who discontinued from the study prior to one year of observation had their last observation for all health-related QoL domains carried forward through the remainder of the year. Similarly, patients who had not completed one year of observation had their health-related QoL data carried forward through one year. If the patient experienced a clinical event within the one year period, the last observation was not carried forward. The Area under the Curve (AUC) value for each domain was computed by multiplying the health-related QoL domain score by the respective duration of that score. Finally, AUC values were aggregated across all subjects within each respective treatment group (atrasentan (10 mg), atrasentan (2.5 mg), and placebo). Aggregated AUC values for each domain were compared for differences between treatment groups using a t-test.

TABLE 2
QUALITY-ADJUSTED TIME-TO-PROGRESSION OF DISEASE
(INTENT TO TREAT DATA)

| | QATTP of Disease | | | | | | P-Value Log Rank Comparison | | |
|---|---|---|---|---|---|---|---|---|---|
| | Median (days) | | | Mean (days) | | | Health-Related QATTP of Disease^{a} | | |
| QoL Domain Score Used for | At. | At. | | At. | At. | | 10 mg | 10 mg | 2.5 mg |
| Adjusting Time-to Progression | (10 mg) | (2.5 mg) | Pl. | (10 mg) | (2.5 mg) | Pl. | vs. 2.5 mg | vs. Pl.^{a} | vs. Pl.^{a} |
| EORTC Physical Functioning | 119 | 118 | 137 | 164 | 172 | 86 | 0.796 | 0.091 | 0.118 |
| EORTC Emotional Functioning | 125 | 133 | 147 | 167 | 177 | 115 | 0.770 | 0.239 | 0.225 |
| EORTC Role Functioning | 123 | 128 | 145 | 180 | 176 | 106 | 0.863 | 0.160 | 0.205 |
| EORTC Social Functioning | 135 | 142 | 151 | 190 | 184 | 112 | 0.722 | 0.106 | 0.209 |
| EORTC Cognitive Functioning | 138 | 151 | 151 | 163 | 180 | 106 | 0.920 | 0.214 | 0.246 |
| EORTC Pain | 127 | 133 | 139 | 172 | 179 | 106 | 0.753 | 0.119 | 0.170 |
| EORTC Fatigue | 104 | 109 | 134 | 153 | 165 | 97 | 0.847 | 0.169 | 0.222 |
| EORTC Nausea & Vomiting | 156 | 162 | 169 | 201 | 195 | 129 | 0.655 | 0.165 | 0.323 |
| EORTC Appetite Loss | 146 | 151 | 161 | 175 | 186 | 118 | 0.616 | 0.157 | 0.309 |
| EORTC Dyspnea | 123 | 141 | 153 | 177 | 173 | 101 | 0.628 | 0.200 | 0.425 |
| EORTC Sleep Disturbance | 123 | 127 | 143 | 158 | 172 | 102 | 0.933 | 0.253 | 0.249 |
| EORTC Diarrhea | 176 | 178 | 169 | 185 | 198 | 129 | 0.733 | 0.201 | 0.270 |
| EORTC Constipation | 132 | 142 | 153 | 189 | 180 | 127 | 0.841 | 0.214 | 0.297 |
| EORTC Global Score | 103 | 104 | 119 | 141 | 159 | 93 | 0.928 | 0.245 | 0.242 |
| FACT Physical Well Being | 127 | 135 | 151 | 184 | 181 | 117 | 0.736 | 0.176 | 0.283 |
| FACT Emotional Well Being | 127 | 133 | 146 | 163 | 180 | 112 | 0.888 | 0.251 | 0.260 |
| FACT Social/Family Well Being | 121 | 126 | 141 | 147 | 160 | 104 | 0.771 | 0.277 | 0.380 |
| FACT Functional Well Being | 98 | 111 | 134 | 140 | 161 | 98 | 0.943 | 0.382 | 0.318 |
| FACT-G | 123 | 129 | 143 | 160 | 172 | 112 | 0.835 | 0.208 | 0.290 |
| FACT-P | 107 | 115 | 122 | 135 | 152 | 87 | 0.770 | 0.202 | 0.273 |
| FACT Total | 117 | 125 | 137 | 152 | 166 | 105 | 0.796 | 0.191 | 0.279 |

^{a}P-Values from Kaplan-Meier log-rank test of differences in health-related QATTP of disease curves.
At. is atrasentan.
Pl. is placebo.

TABLE 3
QUALITY-ADJUSTED TIME-TO-PROGRESSION
(PER PROTOCOL DATA)

| | QATTP of Disease | | | | | | P-Value Log Rank Comparison | | |
|---|---|---|---|---|---|---|---|---|---|
| | Median (days) | | | Mean (days) | | | Health-Related QATTP of Disease^{a} | | |
| QoL Domain Score Used for | At. | At. | | At. | At. | | 10 mg | 10 mg | 2.5 mg |
| Adjusting Time-to-progression | (10 mg) | (2.5 mg) | Pl. | (10 mg) | (2.5 mg) | Pl. | vs. 2.5 mg^{a} | vs. Pl.^{a} | vs. Pl.^{a} |
| EORTC Physical Functioning | 127 | 124 | 85 | 168 | 181 | 128 | 0.932 | 0.019* | 0.014* |
| EORTC Emotional Functioning | 134 | 143 | 110 | 169 | 186 | 135 | 0.856 | 0.038* | 0.021* |
| EORTC Role Functioning | 128 | 142 | 100 | 187 | 185 | 134 | 0.944 | 0.030* | 0.024* |
| EORTC Social Functioning | 141 | 156 | 106 | 196 | 192 | 140 | 0.811 | 0.017* | 0.025* |
| EORTC Cognitive Functioning | 144 | 156 | 106 | 159 | 190 | 142 | 0.944 | 0.040* | 0.031* |
| EORTC Pain | 137 | 142 | 104 | 178 | 188 | 130 | 0.864 | 0.021* | 0.021* |
| EORTC Fatigue | 111 | 127 | 97 | 158 | 174 | 125 | 0.980 | 0.042* | 0.032* |
| EORTC Nausea & Vomiting | 168 | 178 | 127 | 207 | 206 | 158 | 0.792 | 0.029* | 0.042* |
| EORTC Appetite Loss | 146 | 162 | 118 | 179 | 196 | 150 | 0.731 | 0.027* | 0.040* |
| EORTC Dyspnea | 132 | 142 | 98 | 182 | 180 | 145 | 0.738 | 0.060 | 0.119 |
| EORTC Sleep Disturbance | 127 | 137 | 101 | 162 | 179 | 131 | 0.960 | 0.043* | 0.030* |
| EORTC Diarrhea | 184 | 184 | 127 | 188 | 209 | 159 | 0.892 | 0.037* | 0.029* |
| EORTC Constipation | 141 | 151 | 120 | 198 | 190 | 145 | 0.935 | 0.049* | 0.047* |
| EORTC Global Score | 106 | 124 | 90 | 145 | 167 | 113 | 0.975 | 0.057 | 0.038* |
| FACT Physical Well Being | 130 | 148 | 112 | 190 | 191 | 142 | 0.835 | 0.036* | 0.039* |
| FACT Emotional Well Being | 131 | 147 | 107 | 168 | 188 | 137 | 0.995 | 0.053 | 0.035* |
| FACT Social/Family Well Being | 126 | 143 | 102 | 151 | 169 | 131 | 0.912 | 0.059 | 0.049* |
| FACT Functional Well Being | 113 | 111 | 96 | 144 | 168 | 126 | 0.960 | 0.119 | 0.056 |
| FACT-G | 130 | 143 | 105 | 165 | 180 | 135 | 0.957 | 0.047* | 0.040* |
| FACT-P | 111 | 117 | 81 | 139 | 160 | 115 | 0.909 | 0.038* | 0.035* |
| FACT Total | 127 | 135 | 102 | 157 | 174 | 129 | 922 | 0.040* | 0.035* |

^{a}P-Values from Kaplan-Meier log-rank test of differences in health-related QATTP of disease curves.
*Significant at p < 0.05.
At. is atrasentan.
Pl. is placebo.

The data in TABLE 2 show significantly longer (p<0.05) mean health-related QATTP's of disease for all health-related QoL domains except dyspnea, global score, emotional well-being, and social well-being in the 10 mg atrasentan treatment group. In the dyspnea, global score, emotional well-being, and social well-being domains, the trend favored the 10 mg atrasentan treatment group (p<0.10). The 2.5 mg atrasentan treatment group also produced longer mean health-related QATTP of disease. Log rank tests showed these results to be statistically significant for all health-related QoL domains except dyspnea and functional well-being; and there were no statistical differences noted between the atrasentan treatment groups for any health-related QoL domain.

The data in TABLE 3 show both delays and improvement in the mean health-related QATTP's of disease in the both 10 mg and 2.5 mg atrasentan treatment groups.

The AUC analysis results were consistent with the health-related QATTP analyses in TABLES 2 and 3. For the Intent to Treat population (TABLE 2), atrasentan treatment and placebo groups showed no statistical differences. The AUC analysis of the per-protocol population showed strong trends in favor of the atrasentan treatment groups in every health-related QoL domain score when compared to placebo. The responses in the 10 mg and 2.5 mg treatment groups were not statistically differentiable.

The AUC for the health-related QoL domain scores for physical functioning, social functioning, and pain were significantly longer (p<0.05) for atrasentan. Similarly, the 2.5 mg atrasentan group showed significantly improved AUC results except for dyspnea, social/family, functional well-being, and FACT-P domain scores.

The impact of the 10 mg and 2.5 mg atrasentan treatment on the patients' perceived health-related QoL was also addressed. Patient-reported health-related QoL data has validity for two reasons: the perception of health is stated by the patient directly, and multidimensional health-related QoL instruments provide a more complete and balanced assessment of patients' health status. It was found that after adjusting for health-related QoL effects, both 10 mg and 2.5 mg atrasentan therapies offered longer health-related QATTP over placebo in the per protocol population. These gains in the health-related QATTP were robust over a wide range of health-related QoL domain weighting and were consistently observed using the EORTC and FACT as the two health-related QoL instruments. For the intent-to-treat population, there were no statistically significant differences in the QATTP across treatment groups. This finding is consistent with the fact that, for the intent-to-treat population, no statistically significant differences were observed in either the time to disease or PSA progression across treatment groups.

Additional AUC analyses showed that after adjusting for possible bias induced by unequal lengths of follow-up and the staggered entry of subjects, the findings produced by the Kaplan-Meier methods were confirmed.

Thus, both the health-related QoL and the health-related QATTP of disease in patients with prostate cancer are favorably modulated by administration of an ET antagonist, preferably an ET_{A} antagonist such as atrasentan.

What is claimed is:

1. A method for sustaining the health-related quality of life in a patient with prostate cancer comprising administering thereto a therapeutically effective amount of an endothelin receptor antagonist.

2. The method of claim 1 in which the health-related quality of life comprises domains of physical functioning, emotional functioning, social/family functioning, role functioning, cognitive functioning, self-perception, and other domains relating to patients with prostate cancer, the other domains comprising pain, fatigue, nausea and vomiting, change in appetite, dyspnea, sleep disturbance, diarrhea, constipation, urinary function, and change in weight, the domains being assessed by the patient.

3. The method of claim 1 in which the endothelin receptor antagonist is administered at or near the beginning of prostate cancer progression.

4. The method of claim 1 in which the endothelin receptor antagonist is administered toward the end of prostate cancer progression.

5. The method of claim 1 in which the therapeutically effective amount of the endothelin receptor antagonist is between about 1 mg per day to about 25 mg per day.

6. The method of claim 5 in which the endothelin receptor antagonist is administered once or twice per day without missing a day.

7. The method of claim 1 in which the endothelin receptor antagonist is an endothelin A receptor antagonist.

8. The method of claim 7 in which the endothelin A receptor antagonist is a compound having formula (I)-a 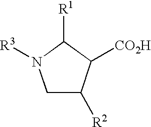
or a compound having formula (I)-a with the relative or absolute stereochemistry shown in the compound having formula (I)-b 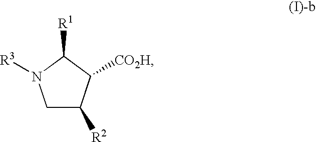
or a therapeutically acceptable salt, prodrug, or salt of prodrug of either, in which
R¹ and R² are independently alkyl, alkenyl, alkynyl, cycloalkyl, aryl, heteroaryl, or alkyl substituted with one cycloalkyl, halo, aryl, heteroaryl, heterocyclyl, —OH, or —O(alkyl) substituent;
R³ is R⁴SO₂R⁵— or R⁴C(O)R⁵—;
R⁴ is alkyl, —(CH₂)alkenyl, —(CH₂)alkynyl, —NR⁶R⁷, alkyl independently substituted with one or two cycloalkyl, aryl, heteroaryl, heterocyclyl, halo, —OH, —O(alkyl), —NH₂, —NH(alkyl), or —N(alkyl)₂ substituents, or alkenyl independently substituted with one or two cycloalkyl, aryl, heteroaryl, heterocyclyl, halo, —OH, —O(alkyl), —NH₂, —NH(alkyl), or —N(alkyl)₂ substituents;
R⁵ is a covalent bond, alkylene, —N(H)(alkylene)-, or —N(alkyl)(alkylene)-, the latter two of which are drawn from left or right, and
R⁶ and R⁷ are independently hydrogen, alkyl, —(CH₂)alkenyl, —(CH₂)alkynyl, cycloalkyl, aryl, or alkyl independently substituted with one or two cycloalkyl, aryl, heteroaryl, heterocyclyl, halo, —OH, —O(alkyl), —OCH₂CF₃, —OCH₂CF₂CF₃, —NH₂, —NH(alkyl), or —N(alkyl)₂ substituents.

9. The method of claim 8 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 2,2-dimethylpentyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

10. The method of claim 8 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 3-fluoro-4-methoxyphenyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

11. The method of claim 8 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 4-methoxyphenyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

12. The method of claim 8 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 2,2-dimethylpentyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

13. The method of claim 8 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 3-fluoro-4-methoxyphenyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

14. The method of claim 8 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 4-methoxyphenyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

15. The method of claim 8 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 2,2-dimethylpentyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

16. The method of claim 8 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 3-fluoro-4-methoxyphenyl; R² is 1,3-benzodioxol-5-yl; and R³ is ((N,N-dibutyl amino)carbonyl)methyl, ((N-butyl-N-(4-(dimethylamino)butyl)amino)carbonyl)methyl, or 2-(N-((pentyl)sulfonyl)-N-propylamino)ethyl.

17. The method of claim 8 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 4-methoxyphenyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

18. The method of claim 8 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 2,2-dimethylpentyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

19. The method of claim 8 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 3-fluoro-4-methoxyphenyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

20. The method of claim 8 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 4-methoxyphenyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

21. The method of claim 8 in which the endothelin A receptor antagonist is
trans,trans-2-(4-methoxyphenyl)-4-(1,3-benzodioxol-5-yl)-1-(((N-propylamino)carbonyl)methyl)pyrrolidine-3-carboxylic acid,
trans,trans-2-(4-methoxyphenyl)-4-(1,3-benzodioxol-5-yl)-1-(aminocarbonyl)methyl)pyrrolidine-3-carboxylic acid,
trans,trans-2-(4-methoxyphenyl)-4-(1,3-benzodioxol-5-yl)-1-(((N-propenyl)carbonyl)methyl)pyrrolidine-3-carboxylic acid,
trans,trans-2-(4-methoxyphenyl)-4-(1,3-benzodioxol-5-yl)-1-(((N-butylamino)carbonyl)methyl)pyrrolidine-3-carboxylic acid,
trans,trans-2-(4-methoxyphenyl)-4-(1,3-benzodioxol-5-yl)-1-(((N-methyl-N-propylamino)carbonyl)methyl)pyrrolidine-3-carboxylic acid,
trans,trans-2-(4-methoxyphenyl)-4-(1,3-benzodioxol-5-yl)-1-(((N-2-methylpropyl)carbonyl)methyl)pyrrolidine-3-carboxylic acid,
trans,trans-2-(4-methoxyphenyl)-4-(1,4-benzodioxol-6-yl)-1-(((N-propylamino)carbonyl)methyl)pyrrolidine-3-carboxylic acid,
trans,trans-2-(4-methoxyphenyl)-4-(1,4-benzodioxol-6-yl)-1-(((N-methyl-N-propylamino)carbonyl)methyl)pyrrolidine-3-carboxylic acid,
trans,trans-2-(4-methoxyphenyl)-4-(1,3-benzodioxol-5-yl)-1-(((N-butyl-N-methylamino)carbonyl)methyl)pyrrolidine-3-carboxylic acid,
trans,trans-2-(4-methoxyphenyl)-4-(1,3-benzodioxol-5-yl)-1-(((N,N-bis(3-methylbutyl)amino)carbonyl)methyl)pyrrolidine-3-carboxylic acid,
trans,trans-2-(4-methoxyphenyl)-4-(1,3-benzodioxol-5-yl)-1(((N,N-dipentylamino)carbonyl)methyl)pyrrolidine-3-carboxylic acid,
trans,trans-2-(4-methoxyphenyl)-4-(1,3-benzodioxol-5-yl)-1-(((N-methyl-N-pentylamino)carbonyl)methyl)pyrrolidine-3-carboxylic acid,
trans,trans-2-(4-methoxyphenyl)-4-(1,3-benzodioxol-5-yl)-1-(((N,N-diisobutylamino)carbonyl)methyl)pyrrolidine-3-carboxylic acid,
trans,trans-2-(4-methoxyphenyl)-4-(1,3-benzodioxol-5-yl)-1-(((N-hexyl-N-methylamino)carbonyl)methyl)pyrrolidine-3-carboxylic acid,
trans,trans-2-(4-methoxyphenyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(((N,N-diethylamino)carbonyl)methyl)pyrrolidine-3-carboxylic acid,
trans,trans-2-(4-methoxyphenyl)-4-(1,3-benzodioxol-5-yl)-1-(((N,N-dipropylamino)carbonyl)methyl)pyrrolidine-3-carboxylic acid,
trans,trans-2-(4-methoxyphenyl)-4-(1,3-benzodioxol-5-yl)-1-(((N-isobutyl-N-methylamino)carbonyl)methyl)pyrrolidine-3-carboxylic acid,
trans,trans-2-(4-methoxyphenyl)-4-(1,3-benzodioxol-5-yl)-1-(2-(N-((isopropyl)sulfonyl)amino)ethyl)pyrrolidine-3-carboxylic acid,
trans,trans-2-(4-methoxyphenyl)-4-(1,3-benzodioxol-5-yl)-1-(((N-butyl-N-ethylamino)carbonyl)methyl)pyrrolidine-3-carboxylic acid,
trans,trans-2-(4-methoxyphenyl)-4-(1,3-benzodioxol-5-yl)-1-(((N-(2,2-dimethylpropyl)-N-methylamino)carbonyl)methyl)pyrrolidine-3-carboxylic acid,
trans,trans-2-(4-methoxyphenyl)-4-(1,3-benzodioxol-5-yl)-1-(2-(N-((butyl)sulfonyl)-N-methylamino)ethyl)pyrrolidine-3-carboxylic acid,
trans,trans-2-(4-methoxyphenyl)-4-(1,3-benzodioxol-5-yl)-1-(2-(N-methyl-N-((propyl)sulfonyl)amino)ethyl)pyrrolidine-3-carboxylic acid,
(2R,3R,4R)-2-(4-methoxyphenyl)-4-(1,3-benzodioxol-5-yl)-1-(((1R)-1-(N,N-dipropylamino)carbonyl)but-1-yl)pyrrolidine-3-carboxylic acid,
(2S,3S,4S)-2-(4-methoxyphenyl)-4-(1,3-benzodioxol-5-yl)-1-(((1R)-1-(N,N-dipropylamino)carbonyl)but-1-yl)pyrrolidine-3-carboxylic acid,
(2S,3S,4S)-2-(4-methoxyphenyl)-4-(1,3-benzodioxol-5-yl)-1-(((1S)-1-(N,N-dipropylamino)carbonyl)but-1-yl)pyrrolidine-3-carboxylic acid,
(2R,3R,4R)-2-(4-methoxyphenyl)-4-(1,3-benzodioxol-5-yl)-1-(((1S)-1-(N,N-dipropylamino)carbonyl)but-1-yl)pyrrolidine-3-carboxylic acid,
trans,trans-2-(4-methoxyphenyl)-4-(1,3-benzodioxol-5-yl)-1-(((N-butyl-N-propylamino)carbonyl)methyl)pyrrolidine-3-carboxylic acid,
trans,trans-2-(4-methoxyphenyl)-4-(1,3-benzodioxol-5-yl)-1-(2-(N,N-dibutylamino)carbonyl)ethyl)pyrrolidine-3-carboxylic acid,
trans,trans-2-(4-methoxyphenyl)-4-(1,3-benzodioxol-5-yl)-1-(((R,S)-2-(N,N-dibutylamino)carbonyl)ethyl)pyrrolidine-3-carboxylic acid,
trans,trans-2-pentyl-4-(1,3-benzodioxol-5-yl)-1-((N,N-dibutylamino)carbonyl)methyl)pyrrolidine-3-carboxylic acid,
trans,trans-2-pentyl-4-(1,3-benzodioxol-5-yl)-1-(2-(N-propyl-N-((propyl)sulfonyl)amino)ethyl)pyrrolidine-3-carboxylic acid,
trans,trans-2-propyl-4-(1,3-benzodioxol-5-yl)-1-(((N,N-dibutylamino)carbonyl)methyl)pyrrolidine-3-carboxylic acid,
(2R,3R,4S)-2-(4-methoxyphenyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(2-(N-butyl-N-((propyl)sulfonyl)amino)ethyl)pyrrolidine-3-carboxylic acid,
trans,trans-2-(4-methoxyphenyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(2-(N-propyl-N-((propyl)sulfonyl)amino)ethyl)pyrrolidine-3-carboxylic acid,
trans,trans-2-(4-methoxyphenyl)-4-(1,3-benzodioxol-5-yl)-1-(2-(N-butyl-N-((butyl)sulfonyl)amino)ethyl)pyrrolidine-3-carboxylic acid,
trans,trans-2-(4-hydroxyphenyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(2-(N-(2-methylpropyl)-N-((propyl)sulfonyl)amino)ethyl)pyrrolidine-3-carboxylic acid,
trans,trans-2-(4-methoxyphenyl)-4-(1,3-benzodioxol-5-yl)-1-(2-(N-((ethyl)sulfonyl)-N-propylamino)ethyl)pyrrolidine-3-carboxylic acid,
trans,trans-2-(4-methoxyphenyl)-4-(1,3-benzodioxol-5-yl)-1-(2-(N-((butyl)sulfonyl)-N-propylamino)ethyl)pyrrolidine-3-carboxylic acid,
trans,trans-2-(isopropyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(2-(N-((pentyl)sulfonyl)-N-propylamino)ethyl)pyrrolidine-3-carboxylic acid,
trans,trans-2-(4-methoxyphenyl)-4-(1,3-benzodioxol-5-yl)-1-(2-((N-(2-methylpropyl)sulfonyl)-N-propylamino)ethyl)pyrrolidine-3-carboxylic acid,
trans,trans-2-(4-methoxyphenyl)-4-(1,3-benzodioxol-5-yl)-1-(2-(N-((heptyl)sulfonyl)-N-propylamino)ethyl)pyrrolidine-3-carboxylic acid,
trans,trans-2-(4-methoxyphenyl)-4-(1,3-benzodioxol-5-yl)-1-(2-N-((hexyl)sulfonyl)-N-propylamino)ethyl)pyrrolidine-3-carboxylic acid,
trans,trans-2-(4-methoxyphenyl)-4-(1,3-benzodioxol-5-yl)-1-(2-(N-((pentyl)sulfonyl)-N-ethylamino)ethyl)pyrrolidine-3-carboxylic acid,
trans,trans-2-(3-fluoro-4-methoxyphenyl)-4-(1,3-benzodioxol-5-yl)-1-(((N,N-dibutylamino)carbonyl)methyl)pyrrolidine-3-carboxylic acid,
trans,trans-2-propyl-4-(1,3-benzodioxol-5-yl)-1-(2-(N-((pentyl)sulfonyl)-N-propylamino)ethyl)pyrrolidine-3-carboxylic acid,
trans,trans-2-(3-fluoro-4-methoxyphenyl)-4-(1,3-benzodioxol-5-yl)-1-(2-(N-((pentyl)sulfonyl)-N-propylamino)ethyl)pyrrolidine-3-carboxylic acid,
trans,trans-2-(3-fluoro-4-methoxyphenyl)-4-(1,3-benzodioxol-5-yl)-1-(2-(N-((butyl)sulfonyl)-N-propylamino)ethyl)pyrrolidine-3-carboxylic acid,
trans,trans-2-(4-methoxyphenyl)-4-(1,3-benzodioxol-5-yl)-1-(3-(N-((pentyl)sulfonyl)-N-propylamino)propyl)pyrrolidine-3-carboxylic acid,
trans,trans-2-butyl-4-(1,3-benzodioxol-5-yl)-1-(((N,N-dibutylamino)carbonyl)methyl)pyrrolidine-3-carboxylic acid,
trans,trans-2-(2-methylbutyl)-4-(1,3-benzodioxol-5-yl)-1-(((N,N-dibutylamino)carbonyl)methyl)pyrrolidine-3-carboxylic acid,
trans,trans-2-(3-methylbutyl)-4-(1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(1,3-benzodioxol-5-yl)-1-(2-(N-((hexyl)sulfonyl)-N-propylamino)ethyl)pyrrolidine-3-carboxylic acid,
trans,trans-2-hexyl-4-(1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(7-methoxy-1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(7-methoxy-1,3-benzodioxol-5-yl)-1-(((N-butyl-N-propylamino)carbonyl)methyl)pyrrolidine-3-carboxylic acid,
trans,trans-2-heptyl-4-(1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(1,3-benzodioxol-5-yl)-1-(2-(N-((3-methylbutyl)sulfonyl)-N-propylamino)ethyl)pyrrolidine-3-carboxylic acid,
trans,trans-2-(3-fluoro-4-methoxyphenyl)-4-(7-methoxy-1,3-benzodioxol-5-yl)-1-(2-(N-((pentyl)sulfonyl)-N-propylamino)ethyl)pyrrolidine-3-carboxylic acid,
trans,trans-2-(3-fluoro-4-methoxyphenyl)-4-(1,3-benzodioxol-5-yl)-1-(2-(N-(2-methylpropyl)-N-((pentyl)sulfonyl)amino)ethyl)pyrrolidine-3-carboxylic acid,
trans,trans-2-(4-methoxyphenyl)-4-(1,3-benzodioxol-5-yl)-1-((N-(non-5-ylamino)carbonyl)methyl)pyrrolidine-3-carboxylic acid,
trans,trans-2-(3-fluoro-4-methoxyphenyl)-4-(1,3-benzodioxol-5-yl)-1-(2-(N-((2-methylpropyl)sulfonyl)-N-propylamino)ethyl)pyrrolidine-3-carboxylic acid,
trans,trans-2-(4-methoxyphenyl)-4-(1,3-benzodioxol-5-yl)-1-(((N,N-dihexylamino)carbonyl)methyl)pyrrolidine-3-carboxylic acid,
trans,trans-2-(4-methoxyphenyl)-4-(1,3-benzodioxol-5-yl)-1-(((N-butyl-N-(hept-4-yl)amino)carbonyl)methyl)pyrrolidine-3-carboxylic acid,
trans,trans-2-(2-propylpentyl)-4-(1,3-benzodioxol-5-yl)-1-(((N,N-dibutylamino)carbonyl)methyl)pyrrolidine-3-carboxylic acid,
trans,trans-2-(3-methylpentyl)-4-(1,3-benzodioxol-5-yl)-1-(((N,N-dibutylamino)carbonyl)methyl)pyrrolidine-3-carboxylic acid,
trans,trans-2-(2-ethylbutyl)-4-(1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(1,3-benzodioxol-5-yl)-1-(2-(N-((butyl)sulfonyl)-N-(2-methylpropyl)amino)ethyl)pyrrolidine-3-carboxylic acid,
trans,trans-2-(3-methyl-(E)-pent-3-en-1-yl)-4-(1,3-benzodioxol-5-yl)-1-(((N,N-dibutylamino)carbonyl)methyl)pyrrolidine-3-carboxylic acid,
trans,trans-2-(2-methylpentyl)-4-(1,3-benzodioxol-5-yl)-1-(((N,N-dibutylamino)carbonyl)methyl)pyrrolidine-3-carboxylic acid,
trans,trans-2-(2,2-dimethylphenyl)-4-(1,3-benzodioxol-5-yl)-1-(((N,N-dibutylamino)carbonyl)methyl)pyrrolidine-3-carboxylic acid,
trans,trans-2-(2,2,4-trimethyl-3-(E)-pent-3-enyl)-4-(1,3-benzodioxol-5-yl)-1-(((N,N-dibutylamino)carbonyl)methyl)pyrrolidine-3-carboxylic acid,
trans,trans-2-(2,2,-dimethylpentyl)-4-(7-methoxy-1,3-benzodioxol-5-yl)-1-(((N,N-dibutylamino)carbonyl)methyl)pyrrolidine-3-carboxylic acid,
trans,trans-2-(2,2-dimethyl-(E)-pent-3-enyl)-4-(7-methoxy-1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(((N-(4-aminobutyl)-N-butylamino)carbonyl)methyl)pyrrolidine-3-carboxylic acid,
trans,trans-2-(4-methoxyphenyl)-4-(1,3-benzodioxol-5-yl)-1-(((N-butyl-N-(4-(dimethylamino)butyl)amino)carbonyl)methyl)pyrrolidine-3-carboxylic acid,
(2R,3R,4S)-2-(3-fluoro-4-methoxyphenyl)-4-(1,3-benzodioxol-5-yl)-1-(2-(N-((pentyl)sulfonyl)-N-propylamino)ethyl)pyrrolidine-3-carboxylic acid,
(2S,3R,4S)-2-(2,2-dimethylpentyl)-4-(7-methoxy-1,3-benzodioxol-5-yl)-1-(((N,N-dibutylamino)carbonyl)methyl)pyrrolidine-3-carboxylic acid,
(2S,3R,4S)-2-(2,2-dimethylpentyl)-4-(1,3-benzodioxol-5-yl)-1-(((N,N-dibutylamino)carbonyl)methyl)pyrrolidine-3-carboxylic acid,
(2S,3R,4S)-2-(4-methoxyphenyl)-4-(1,3-benzodioxol-5-yl)-1-(((N-butyl-N-(4-(dimethylamino)butyl)amino)carbonyl)methyl)pyrrolidine-3-carboxylic acid,
(2S,3R,4S)-2-(2,2-dimethyl-(E)-pent-3-enyl)-4-(1,3-benzodioxol-5-yl)-1-(((N,N-dibutylamino)carbonyl)methyl)pyrrolidine-3-carboxylic acid, or
(2S,3R,4S)-2-(2,2-dimethyl-(E)-pent-3-enyl)-4-(7-methoxy-1,3-benzodioxol-5-yl)-1-(((N,N-dibutylamino)carbonyl)methyl)pyrrolidine-3-carboxylic acid, or a therapeutically acceptable salt, prodrug, or salt of a prodrug thereof.

22. The method of claim 21 in which the endothelin A receptor antagonist is
(2R,3R,4S)-2-(4-methoxyphenyl)-4-(1,3-benzodioxol-5-yl)-1-(((N,N-dibutylamino)carbonyl)methyl)pyrrolidine-3-carboxylic acid,
(2R,3R,4S)-2-(3-fluoro-4-methoxyphenyl)-4-(1,3-benzodioxol-5-yl)-1-(2-(N-((pentyl)sulfonyl)-N-propylamino)ethyl)pyrrolidine-3-carboxylic acid,
(2S,3R,4S)-2-(2,2-dimethylpentyl)-4-(7-methoxy-1,3-benzodioxol-5-yl)-1-(((N,N-dibutylamino)carbonyl)methyl)pyrrolidine-3-carboxylic acid,
(2S,3R,4S)-2-(2,2-dimethylpentyl)-4-(1,3-benzodioxol-5-yl)-1-(((N,N-dibutylamino)carbonyl)methyl)pyrrolidine-3-carboxylic acid,
(2S,3R,4S)-2-(4-methoxyphenyl)-4-(1,3-benzodioxol-5-yl)-1-(((N-butyl-N-(4-(dimethylamino)butyl)amino)carbonyl)methyl)pyrrolidine-3-carboxylic acid,
(2S,3R,4S)-2-(2,2-dimethyl-(E)-pent-3-enyl)-4-(1,3-benzodioxol-5-yl)-1-(((N,N-dibutylamino)carbonyl)methyl)pyrrolidine-3-carboxylic acid, or
(2S,3R,4S)-2-(2,2-dimethyl-(E)-pent-3-enyl)-4-(7-methoxy-1,3-benzodioxol-5-yl)-1-(((N,N-dibutylamino)carbonyl)methyl)pyrrolidine-3-carboxylic acid, or a therapeutically acceptable salt, prodrug, or salt of a prodrug thereof.

23. The method of claim 22 in which the endothelin A receptor antagonist is
(2R,3R,4S)-2-(4-methoxyphenyl)-4-(1,3-benzodioxol-5-yl)-1-(((N,N-dibutylamino)carbonyl)methyl)pyrrolidine-3-carboxylic acid, or a therapeutically acceptable salt, prodrug, or salt of a prodrug thereof.

24. A method for improving the health-related quality of life in a patient with prostate cancer comprising administering thereto a therapeutically effective amount of an endothelin receptor antagonist.

25. The method of claim 24 in which the health-related quality of life comprises domains of physical functioning, emotional functioning, social/family functioning, role functioning, cognitive functioning, self-perception, and other domains relating to patients with prostate cancer, the other domains comprising pain, fatigue, nausea and vomiting, change in appetite, dyspnea, sleep disturbance, diarrhea, constipation, urinary function, and change in weight, the domains being assessed by the patient.

26. The method of claim 24 in which the endothelin receptor antagonist is administered at or near the beginning of prostate cancer progression.

27. The method of claim 24 in which the endothelin receptor antagonist is administered toward the end of prostate cancer progression.

28. The method of claim 24 in which the therapeutically effective amount of the endothelin receptor antagonist is between about 1 mg per day to about 25 mg per day.

29. The method of claim 28 in which the endothelin receptor antagonist is administered once or twice per day without missing a day.

30. The method of claim 24 in which the endothelin receptor antagonist is an endothelin A receptor antagonist.

31. The method of claim 30 in which the endothelin A receptor antagonist is a compound having formula (I)-a 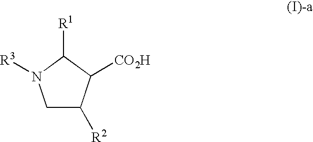
or a compound having formula (I)-a with the relative or absolute stereochemistry shown in the compound having formula (I)-b 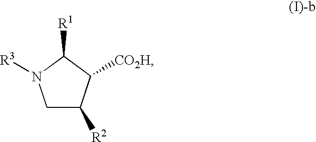
or a therapeutically acceptable salt, prodrug, or salt of prodrug of either, in which
R¹ and R² are independently alkyl, alkenyl, alkynyl, cycloalkyl, aryl, heteroaryl, or alkyl substituted with one cycloalkyl, halo, aryl, heteroaryl, heterocyclyl, —OH, or —O(alkyl) substituent;
R³ is R⁴SO₂R⁵— or R⁴C(O)R⁵—;
R⁴ is alkyl, —(CH₂)alkenyl, —(CH₂)alkynyl, —NR⁶R⁷, alkyl independently substituted with one or two cycloalkyl, aryl, heteroaryl, heterocyclyl, halo, —OH, —O(alkyl), ^{NH}₂, —NH(alkyl), or —N(alkyl)₂ substituents, or alkenyl independently substituted with one or two cycloalkyl, aryl, heteroaryl, heterocyclyl, halo, —OH, —O(alkyl), —NH₂, —NH(alkyl), or —N(alkyl)₂ substituents;
R⁵ is a covalent bond, alkylene, —N(H)(alkylene)-, or —N(alkyl)(alkylene)-, the latter two of which are drawn from left or right, and
R⁶ and R⁷ are independently hydrogen, alkyl, —(CH₂)alkenyl, —(CH₂)alkynyl, cycloalkyl, aryl, or alkyl independently substituted with one or two cycloalkyl, aryl, heteroaryl, heterocyclyl, halo, —OH, —O(alkyl), —OCH₂CF₃, —OCH₂CF₂CF₃, —NH₂, —NH(alkyl), or —N(alkyl)₂ substituents.

32. The method of claim 31 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 2,2-dimethylpentyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

33. The method of claim 31 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 3-fluoro-4-methoxyphenyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

34. The method of claim 31 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 4-methoxyphenyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

35. The method of claim 31 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 2,2-dimethylpentyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

36. The method of claim 31 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 3-fluoro-4-methoxyphenyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

37. The method of claim 31 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 4-methoxyphenyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

38. The method of claim 31 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 2,2-dimethylpentyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

39. The method of claim 31 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 3-fluoro-4-methoxyphenyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

40. The method of claim 31 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 4-methoxyphenyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-(pentyl)sulfonyl)-N-propylamino)ethyl.

41. The method of claim 31 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 2,2-dimethylpentyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

42. The method of claim 31 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 3-fluoro-4-methoxyphenyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

43. The method of claim 31 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 4-methoxyphenyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

44. The method of claim 31 in which the endothelin A receptor antagonist is
trans,trans-2-(4-methoxyphenyl)-4-(1,3-benzodioxol-5-yl)-1-(((N-propylamino)carbonyl)methyl)pyrrolidine-3-carboxylic acid,
trans,trans-2-(4-methoxyphenyl)-4-(1,3-benzodioxol-5-yl)-1-(aminocarbonyl)methyl)pyrrolidine-3-carboxylic acid,
trans,trans-2-(4-methoxyphenyl)-4-(1,3-benzodioxol-5-yl)-1-(((N-propenyl)carbonyl)methyl)pyrrolidine-3-carboxylic acid,
trans,trans-2-(4-methoxyphenyl)-4-(1,3-benzodioxol-5-yl)-1-(((N-butylamino)carbonyl)methyl)pyrrolidine-3-carboxylic acid,
trans,trans-2-(4-methoxyphenyl)-4-(1,3-benzodioxol-5-yl)-1-(((N-methyl-N-propylamino)carbonyl)methyl)pyrrolidine-3-carboxylic acid,
trans,trans-2-(4-methoxyphenyl)-4-(1,3-benzodioxol-5-yl)-1-(((N-2-methylpropyl)carbonyl)methyl)pyrrolidine-3-carboxylic acid,
trans,trans-2-(4-methoxyphenyl)-4-(1,4-benzodioxol-6-yl)-1-(((N-propylamino)carbonyl)methyl)pyrrolidine-3-carboxylic acid,
trans,trans-2-(4-methoxyphenyl)-4-(1,4-benzodioxol-6-yl)-1-(((N-methyl-N-propylamino)carbonyl)methyl)pyrrolidine-3-carboxylic acid,
trans,trans-2-(4-methoxyphenyl)-4-(1,3-benzodioxol-5-yl)-1-(((N-butyl-N-methylamino)carbonyl)methyl)pyrrolidine-3-carboxylic acid,
trans,trans-2-(4-methoxyphenyl)-4-(1,3-benzodioxol-5-yl)-1-(((N,N-bis(3-methylbutyl)amino)carbonyl)methyl)pyrrolidine-3-carboxylic acid,
trans,trans-2-(4-methoxyphenyl)-4-(1,3-benzodioxol-5-yl)-1-(((N,N-dipentylamino)carbonyl)methyl)pyrrolidine-3-carboxylic acid,
trans,trans-2-(4-methoxyphenyl)-4-(1,3-benzodioxol-5-yl)-1-(((N-methyl-N-pentylamino)carbonyl)methyl)pyrrolidine-3-carboxylic acid,
trans,trans-2-(4-methoxyphenyl)-4-(1,3-benzodioxol-5-yl)-1-(((N,N-diisobutylamino)carbonyl)methyl)pyrrolidine-3-carboxylic acid,
trans,trans-2-(4-methoxyphenyl)-4-(1,3-benzodioxol-5-yl)-1-(((N-hexyl-N-methylamino)carbonyl)methyl)pyrrolidine-3-carboxylic acid,
trans,trans-2-(4-methoxyphenyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(((N,N-diethylamino)carbonyl)methyl)pyrrolidine-3-carboxylic acid,
trans,trans-2-(4-methoxyphenyl)-4-(1,3-benzodioxol-5-yl)-1-(((N,N-dipropylamino)carbonyl)methyl)pyrrolidine-3-carboxylic acid,
trans,trans-2-(4-methoxyphenyl)-4-(1,3-benzodioxol-5-yl)-1-(((N-isobutyl-N-methylamino)carbonyl)methyl)pyrrolidine-3-carboxylic acid,
trans,trans-2-(4-methoxyphenyl)-4-(1,3-benzodioxol-5-yl)-1-(2-(N-((isopropyl)sulfonyl)amino)ethyl)pyrrolidine-3-carboxylic acid,
trans,trans-2-(4-methoxyphenyl)-4-(1,3-benzodioxol-5-yl)-1-(((N-butyl-N-ethylamino)carbonyl)methyl)pyrrolidine-3-carboxylic acid,
trans,trans-2-(4-methoxyphenyl)-4-(1,3-benzodioxol-5-yl)-1-(((N-(2,2-dimethylpropyl)-N-methylamino)carbonyl)methyl)pyrrolidine-3-carboxylic acid,
trans,trans-2-(4-methoxyphenyl)-4-(1,3-benzodioxol-5-yl)-1-(2-(N-((butyl)sulfonyl)-N-methylamino)ethyl)pyrrolidine-3-carboxylic acid,
trans,trans-2-(4-methoxyphenyl)-4-(1,3-benzodioxol-5-yl)-1-(2-(N-methyl-N-((propyl)sulfonyl)amino)ethyl)pyrrolidine-3-carboxylic acid,
(2R,3R,4R)-2-(4-methoxyphenyl)-4-(1,3-benzodioxol-5-yl)-1-(((1R)-1-(N,N-dipropylamino)carbonyl)but-1-yl)pyrrolidine-3-carboxylic acid,
(2S,3S,4S)-2-(4-methoxyphenyl)-4-(1,3-benzodioxol-5-yl)-1-(((1R)-1-(N,N-dipropylamino)carbonyl)but-1-yl)pyrrolidine-3-carboxylic acid,
(2S,3S,4S)-2-(4-methoxyphenyl)-4-(1,3-benzodioxol-5-yl)-1-(((1S)-1-(N,N-dipropylamino)carbonyl)but-1-yl)pyrrolidine-3-carboxylic acid,
(2R,3R,4R)-2-(4-methoxyphenyl)-4-(1,3-benzodioxol-5-yl)-1-(((1S)-1-(N,N-dipropylamino)carbonyl)but-1-yl)pyrrolidine-3-carboxylic acid,
trans,trans-2-(4-methoxyphenyl)-4-(1,3-benzodioxol-5-yl)-1-(((N-butyl-N-propylamino)carbonyl)methyl)pyrrolidine-3-carboxylic acid,
trans,trans-2-(4-methoxyphenyl)-4-(1,3-benzodioxol-5-yl)-1-(2-(N,N-dibutylamino)carbonyl)ethyl)pyrrolidine-3-carboxylic acid,
trans,trans-2-(4-methoxyphenyl)-4-(1,3-benzodioxol-5-yl)-1-(((R,S)-2-(N,N-dibutylamino)carbonyl)ethyl)pyrrolidine-3-carboxylic acid,
trans,trans-2-pentyl-4-(1,3-benzodioxol-5-yl)-1-((N,N-dibutylamino)carbonyl)methyl)pyrrolidine-3-carboxylic acid,
trans,trans-2-pentyl4-(1,3-benzodioxol-5-yl)-1-(2-(N-propyl-N-((propyl)sulfonyl)amino)ethyl)pyrrolidine-3-carboxylic acid,
trans,trans-2-propyl-4-(1,3-benzodioxol-5-yl)-1-(((N,N-dibutylamino)carbonyl)methyl)pyrrolidine-3-carboxylic acid,
(2R,3R,4S)-2-(4-methoxyphenyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(2-(N-butyl-N-((propyl)sulfonyl)amino)ethyl)pyrrolidine-3-carboxylic acid,
trans,trans-2-(4-methoxyphenyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(2-(N-propyl-N-((propyl)sulfonyl)amino)ethyl)pyrrolidine-3-carboxylic acid,
trans,trans-2-(4-methoxyphenyl)-4-(1,3-benzodioxol-5-yl)-1-(2-(N-butyl-N-((butyl)sulfonyl)amino)ethyl)pyrrolidine-3-carboxylic acid,
trans,trans-2-(4-hydroxyphenyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(2-(N-(2-methylpropyl)-N-((propyl)sulfonyl)amino)ethyl)pyrrolidine-3-carboxylic acid,
trans,trans-2-(4-methoxyphenyl)-4-(1,3-benzodioxol-5-yl)-1-(2-(N-((ethyl)sulfonyl)-N-propylamino)ethyl)pyrrolidine-3-carboxylic acid,
trans,trans-2-(4-methoxyphenyl)-4-(1,3-benzodioxol-5-yl)-1-(2-(N-((butyl)sulfonyl)-N-propylamino)ethyl)pyrrolidine-3-carboxylic acid,
trans,trans-2-(isopropyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(2-(N-((pentyl)sulfonyl)-N-propylamino)ethyl)pyrrolidine-3-carboxylic acid,
trans,trans-2-(4-methoxyphenyl)-4-(1,3-benzodioxol-5-yl)-1-(2-((N-(2-methylpropyl)sulfonyl)-N-propylamino)ethyl)pyrrolidine-3-carboxylic acid,
trans,trans-2-(4-methoxyphenyl)-4-(1,3-benzodioxol-5-yl)-1-(2-(N-((heptyl)sulfonyl)-N-propylamino)ethyl)pyrrolidine-3-carboxylic acid,
trans,trans-2-(4-methoxyphenyl)-4-(1,3-benzodioxol-5-yl)-1-(2-(N-((hexyl)sulfonyl)-N-propylamino)ethyl)pyrrolidine-3-carboxylic acid,
trans,trans-2-(4-methoxyphenyl)-4-(1,3-benzodioxol-5-yl)-1-(2-(N-((pentyl)sulfonyl)-N-ethylamino)ethyl)pyrrolidine-3-carboxylic acid,
trans,trans-2-(3-fluoro-4-methoxyphenyl)-4-(1,3-benzodioxol-5-yl)-1-(((N,N-dibutylamino)carbonyl)methyl)pyrrolidine-3-carboxylic acid,
trans,trans-2-propyl-4-(1,3-benzodioxol-5-yl)-1-(2-(N-((pentyl)sulfonyl)-N-propylamino)ethyl)pyrrolidine-3-carboxylic acid,
trans,trans-2-(3-fluoro-4-methoxyphenyl)-4-(1,3-benzodioxol-5-yl)-1-(2-(N-((pentyl)sulfonyl)-N-propylamino)ethyl)pyrrolidine-3-carboxylic acid,
trans,trans-2-(3-fluoro-4-methoxyphenyl)-4-(1,3-benzodioxol-5-yl)-1-(2-(N-((butyl)sulfonyl)-N-propylamino)ethyl)pyrrolidine-3-carboxylic acid,
trans,trans-2-(4-methoxyphenyl)-4-(1,3-benzodioxol-5-yl)-1-(3-(N-((pentyl)sulfonyl)-N-propylamino)propyl)pyrrolidine-3-carboxylic acid,
trans,trans-2-butyl-4-(1,3-benzodioxol-5-yl)-1-(((N,N-dibutylamino)carbonyl)methyl)pyrrolidine-3-carboxylic acid,
trans,trans-2-(2-methylbutyl)-4-(1,3-benzodioxol-5-yl)-1-(((N,N-dibutylamino)carbonyl)methyl)pyrrolidine-3-carboxylic acid,
trans,trans-2-(3-methylbutyl)-4-(1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(1,3-benzodioxol-5-yl)-1-(2-(N-((hexyl)sulfonyl)-N-propylamino)ethyl)pyrrolidine-3-carboxylic acid,
trans,trans-2-hexyl-4-(1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(7-methoxy-1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(7-methoxy-1,3-benzodioxol-5-yl)-1-(((N-butyl-N-propylamino)carbonyl)methyl)pyrrolidine-3-carboxylic acid,
trans,trans-2-heptyl-4-(1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(1,3-benzodioxol-5-yl)-1-(2-(N-((3-methylbutyl)sulfonyl)-N-propylamino)ethyl)pyrrolidine-3-carboxylic acid,
trans,trans-2-(3-fluoro-4-methoxyphenyl)-4-(7-methoxy-1,3-benzodioxol-5-yl)-1-(2-(N-((pentyl)sulfonyl)-N-propylamino)ethyl)pyrrolidine-3-carboxylic acid,
trans,trans-2-(3-fluoro-4-methoxyphenyl)-4-(1,3-benzodioxol-5-yl)-1-(2-(N-(2-methylpropyl)-N-((pentyl)sulfonyl)amino)ethyl)pyrrolidine-3-carboxylic acid,
trans,trans-2-(4-methoxyphenyl)-4-(1,3-benzodioxol-5-yl)-1-((N-(non-5-ylamino)carbonyl)methyl)pyrrolidine-3-carboxylic acid,
trans,trans-2-(3-fluoro-4-methoxyphenyl)-4-(1,3-benzodioxol-5-yl)-1-(2-(N-((2-methylpropyl)sulfonyl)-N-propylamino)ethyl)pyrrolidine-3-carboxylic acid,
trans,trans-2-(4-methoxyphenyl)-4-(1,3-benzodioxol-5-yl)-1-(((N,N-dihexylamino)carbonyl)methyl)pyrrolidine-3-carboxylic acid,
trans,trans-2-(4-methoxyphenyl)-4-(1,3-benzodioxol-5-yl)-1-(((N-butyl-N-(hept-4-yl)amino)carbonyl)methyl)pyrrolidine-3-carboxylic acid,
trans,trans-2-(2-propylpentyl)-4-(1,3-benzodioxol-5-yl)-1-(((N,N-dibutylamino)carbonyl)methyl)pyrrolidine-3-carboxylic acid,
trans,trans-2-(3-methylpentyl)-4-(1,3-benzodioxol-5-yl)-1-(((N,N-dibutylamino)carbonyl)methyl)pyrrolidine-3-carboxylic acid,
trans,trans-2-(2-ethylbutyl)-4-(1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(1,3-benzodioxol-5-yl)-1-(2-(N-((butyl)sulfonyl)-N-(2-methylpropyl)amino)ethyl)pyrrolidine-3-carboxylic acid,
trans,trans-2-(3-methyl-(E)-pent-3-en-1-yl)-4-(1,3-benzodioxol-5-yl)-1-(((N,N-dibutylamino)carbonyl)methyl)pyrrolidine-3-carboxylic acid,
trans,trans-2-(2-methylpentyl)-4-(1,3-benzodioxol-5-yl)-1-(((N,N-dibutylamino)carbonyl)methyl)pyrrolidine-3-carboxylic acid,
trans,trans-2-(2,2-dimethylphenyl)-4-(1,3-benzodioxol-5-yl)-1-(((N,N-dibutylamino)carbonyl)methyl)pyrrolidine-3-carboxylic acid,
trans,trans-2-(2,2,4-trimethyl-3-(E)-pent-3-enyl)-4-(1,3-benzodioxol-5-yl)-1-(((N,N-dibutylamino)carbonyl)methyl)pyrrolidine-3-carboxylic acid,
trans,trans-2-(2,2,-dimethylpentyl)-4-(7-methoxy-1,3-benzodioxol-5-yl)-1-(((N,N-dibutylamino)carbonyl)methyl)pyrrolidine-3-carboxylic acid,
trans,trans-2-(2,2-dimethyl-(E)-pent-3-enyl)-4-(7-methoxy-1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(((N-(4-aminobutyl)-N-butylamino)carbonyl)methyl)pyrrolidine-3-carboxylic acid,
trans,trans-2-(4-methoxyphenyl)-4-(1,3-benzodioxol-5-yl)-1-(((N-butyl-N-(4-(dimethylamino)butyl)amino)carbonyl)methyl)pyrrolidine-3-carboxylic acid,
(2R,3R,4S)-2-(3-fluoro-4-methoxyphenyl)-4-(1,3-benzodioxol-5-yl)-1-(2-(N-((pentyl)sulfonyl)-N-propylamino)ethyl)pyrrolidine-3-carboxylic acid,
(2S,3R,4S)-2-(2,2-dimethylpentyl)-4-(7-methoxy-1,3-benzodioxol-5-yl)-1-(((N,N-dibutylamino)carbonyl)methyl)pyrrolidine-3-carboxylic acid,
(2S,3R,4S)-2-(2,2-dimethylpentyl)-4-(1,3-benzodioxol-5-yl)-1-(((N,N-dibutylamino)carbonyl)methyl)pyrrolidine-3-carboxylic acid,
(2S,3R,4S)-2-(4-methoxyphenyl)-4-(1,3-benzodioxol-5-yl)-1-(((N-butyl-N-(4-(dimethylamino)butyl)amino)carbonyl)methyl)pyrrolidine-3-carboxylic acid,
(2S,3R,4S)-2-(2,2-dimethyl-(E)-pent-3-enyl)-4-(1,3-benzodioxol-5-yl)-1-(((N,N-dibutylamino)carbonyl)methyl)pyrrolidine-3-carboxylic acid, or
(2S,3R,4S)-2-(2,2-dimethyl-(E)-pent-3-enyl)-4-(7-methoxy-1,3-benzodioxol-5-yl)-1-(((N,N-dibutylamino)carbonyl)methyl)pyrrolidine-3-carboxylic acid, or a therapeutically acceptable salt, prodrug, or salt of a prodrug thereof.

45. The method of claim 44 in which the endothelin A receptor antagonist is
(2R,3R,4S)-2-(4-methoxyphenyl)-4-(1,3-benzodioxol-5-yl)-1-(((N,N-dibutylamino)carbonyl)methyl)pyrrolidine-3-carboxylic acid,
(2R,3R,4S)-2-(3-fluoro-4-methoxyphenyl)-4-(1,3-benzodioxol-5-yl)-1-(2-(N-((pentyl)sulfonyl)-N-propylamino)ethyl)pyrrolidine-3-carboxylic acid,
(2S,3R,4S)-2-(2,2-dimethylpentyl)-4-(7-methoxy-1,3-benzodioxol-5-yl)-1-(((N,N-dibutylamino)carbonyl)methyl)pyrrolidine-3-carboxylic acid,
(2S,3R,4S)-2-(2,2-dimethylpentyl)-4-(1,3-benzodioxol-5-yl)-1-(((N,N-dibutylamino)carbonyl)methyl)pyrrolidine-3-carboxylic acid,
(2S,3R,4S)-2-(4-methoxyphenyl)-4-(1,3-benzodioxol-5-yl)-1-(((N-butyl-N-(4-(dimethylamino)butyl)amino)carbonyl)methyl)pyrrolidine-3-carboxylic acid,
(2S,3R,4S)-2-(2,2-dimethyl-(E)-pent-3-enyl)-4-(1,3-benzodioxol-5-yl)-1-(((N,N-dibutylamino)carbonyl)methyl)pyrrolidine-3-carboxylic acid, or
(2S,3R,4S)-2-(2,2-dimethyl-(E)-pent-3-enyl)-4-(7-methoxy-1,3-benzodioxol-5-yl)-1-(((N,N-dibutylamino)carbonyl)methyl)pyrrolidine-3-carboxylic acid, or a therapeutically acceptable salt, prodrug, or salt of a prodrug thereof.

46. The method of claim 45 in which the endothelin A receptor antagonist is
(2R,3R,4S)-2-(4-methoxyphenyl)-4-(1,3-benzodioxol-5-yl)-1-(((N,N-dibutylamino)carbonyl)methyl)pyrrolidine-3-carboxylic acid, or a therapeutically acceptable salt, prodrug, or salt of a prodrug thereof.

47. A method for sustaining the health-related quality-adjusted time-to-progression of disease in a patient with prostate cancer comprising administering thereto a therapeutically effective amount of an endothelin receptor antagonist.

48. The method of claim 47 in which the health-related quality of life comprises domains of physical functioning, emotional functioning, social/family functioning, role functioning, cognitive functioning, self-perception, and other domains relating to patients with prostate cancer, the other domains comprising pain, fatigue, nausea and vomiting, change in appetite, dyspnea, sleep disturbance, diarrhea, constipation, urinary function, and change in weight, the domains being assessed by the patient.

49. The method of claim 47 in which the endothelin receptor antagonist is administered at or near the beginning of prostate cancer progression.

50. The method of claim 47 in which the endothelin receptor antagonist is administered toward the end of prostate cancer progression.

51. The method of claim 47 in which the therapeutically effective amount of the endothelin receptor antagonist is between about 1 mg per day to about 25 mg per day.

52. The method of claim 51 in which the endothelin receptor antagonist is administered once or twice per day without missing a day.

53. The method of claim 47 in which the endothelin receptor antagonist is an endothelin A receptor antagonist.

54. The method of claim 53 in which the endothelin A receptor antagonist is a compound having formula (I)-a 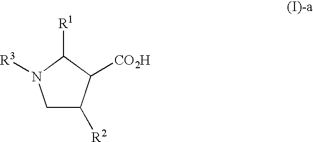
or a compound having formula (I)-a with the relative or absolute stereochemistry shown in the compound having formula (I)-b 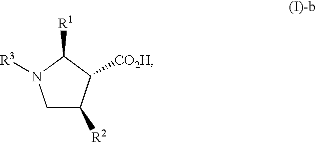
or a therapeutically acceptable salt, prodrug, or salt of prodrug of either, in which
R¹ and R² are independently alkyl, alkenyl, alkynyl, cycloalkyl, aryl, heteroaryl, or alkyl substituted with one cycloalkyl, halo, aryl, heteroaryl, heterocyclyl, —OH, or —O(alkyl) substituent;
R³ is R⁴SO₂R⁵— or R⁴C(O)R⁵—;
R⁴ is alkyl, —(CH₂)alkenyl, —(CH₂)alkynyl, —NR⁶R⁷, alkyl independently substituted with one or two cycloalkyl, aryl, heteroaryl, heterocyclyl, halo, —OH, —O(alkyl), —NH₂, —NH(alkyl), or —N(alkyl)₂ substituents, or alkenyl independently substituted with one or two cycloalkyl, aryl, heteroaryl, heterocyclyl, halo, —OH, —O(alkyl), —NH₂, —NH(alkyl), or —N(alkyl)₂ substituents;
R⁵ is a covalent bond, alkylene, —N(H)(alkylene)-, or —N(alkyl)(alkylene)-, the latter two of which are drawn from left or right, and
R⁶ and R⁷ are independently hydrogen, alkyl, —(CH₂)alkenyl, —(CH₂)alkynyl, cycloalkyl, aryl, or alkyl independently substituted with one or two cycloalkyl, aryl, heteroaryl, heterocyclyl, halo, —OH, —O(alkyl), —OCH₂CF₃, —OCH₂CF₂CF₃, —NH₂, —NH(alkyl), or —N(alkyl)₂ substituents.

55. The method of claim 54 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 2,2-dimethylpentyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

56. The method of claim 54 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 3-fluoro-4-methoxyphenyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

57. The method of claim 54 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 4-methoxyphenyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

58. The method of claim 54 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 2,2-dimethylpentyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

59. The method of claim 54 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 3-fluoro-4-methoxyphenyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-N-((pentyl)sulfonyl)-N-propylamino)ethyl.

60. The method of claim 54 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 4-methoxyphenyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

61. The method of claim 54 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 2,2-dimethylpentyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

62. The method of claim 54 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 3-fluoro-4-methoxyphenyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

63. The method of claim 54 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 4-methoxyphenyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

64. The method of claim 54 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 2,2-dimethylpentyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

65. The method of claim 54 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 3-fluoro-4-methoxyphenyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

66. The method of claim 54 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 4-methoxyphenyl; R2 is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

67. The method of claim 54 in which the endothelin A receptor antagonist is
trans,trans-2-(4-methoxyphenyl)-4-(1,3-benzodioxol-5-yl)-1-(((N-propylamino)carbonyl)methyl)pyrrolidine-3-carboxylic acid,
trans,trans-2-(4-methoxyphenyl)-4-(1,3-benzodioxol-5-yl)-1-(aminocarbonyl)methyl)pyrrolidine-3-carboxylic acid,
trans,trans-2-(4-methoxyphenyl)-4-(1,3-benzodioxol-5-yl)-1-(((N-propenyl)carbonyl)methyl)pyrrolidine-3-carboxylic acid,
trans,trans-2-(4-methoxyphenyl)-4-(1,3-benzodioxol-5-yl)-1-(((N-butylamino)carbonyl)methyl)pyrrolidine-3-carboxylic acid,
trans,trans-2-(4-methoxyphenyl)-4-(1,3-benzodioxol-5-yl)-1-(((N-methyl-N-propylamino)carbonyl)methyl)pyrrolidine-3-carboxylic acid,
trans,trans-2-(4-methoxyphenyl)-4-(1,3-benzodioxol-5-yl)-1-(((N-2-methylpropyl)carbonyl)methyl)pyrrolidine-3-carboxylic acid,
trans,trans-2-(4-methoxyphenyl)-4-(1,4-benzodioxol-6-yl)-1-(((N-propylamino)carbonyl)methyl)pyrrolidine-3-carboxylic acid,
trans,trans-2-(4-methoxyphenyl)-4-(1,4-benzodioxol-6-yl)-1-(((N-methyl-N-propylamino)carbonyl)methyl)pyrrolidine-3-carboxylic acid,
trans,trans-2-(4-methoxyphenyl)-4-(1,3-benzodioxol-5-yl)-1-(((N-butyl-N-methylamino)carbonyl)methyl)pyrrolidine-3-carboxylic acid,
trans,trans-2-(4-methoxyphenyl)-4-(1,3-benzodioxol-5-yl)-1-(((N,N-bis(3-methylbutyl)amino)carbonyl)methyl)pyrrolidine-3-carboxylic acid,
trans,trans-2-(4-methoxyphenyl)-4-(1,3-benzodioxol-5-yl)-1-(((N,N-dipentylamino)carbonyl)methyl)pyrrolidine-3-carboxylic acid,
trans,trans-2-(4-methoxyphenyl)-4-(1,3-benzodioxol-5-yl)-1-(((N-methyl-N-pentylamino)carbonyl)methyl)pyrrolidine-3-carboxylic acid,
trans,trans-2-(4-methoxyphenyl)-4-(1,3-benzodioxol-5-yl)-1-(((N,N-diisobutylamino)carbonyl)methyl)pyrrolidine-3-carboxylic acid,
trans,trans-2-(4-methoxyphenyl)-4-(1,3-benzodioxol-5-yl)-1-(((N-hexyl-N-methylamino)carbonyl)methyl)pyrrolidine-3-carboxylic acid,
trans,trans-2-(4-methoxyphenyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(((N,N-diethylamino)carbonyl)methyl)pyrrolidine-3-carboxylic acid,
trans,trans-2-(4-methoxyphenyl)-4-(1,3-benzodioxol-5-yl)-1-(((N,N-dipropylamino)carbonyl)methyl)pyrrolidine-3-carboxylic acid,
trans,trans-2-(4-methoxyphenyl)-4-(1,3-benzodioxol-5-yl)-1-(((N-isobutyl-N-methylamino)carbonyl)methyl)pyrrolidine-3-carboxylic acid,
trans,trans-2-(4-methoxyphenyl)-4-(1,3-benzodioxol-5-yl)-1-(2-(N-((isopropyl)sulfonyl)amino)ethyl)pyrrolidine-3-carboxylic acid,
trans,trans-2-(4-methoxyphenyl)-4-(1,3-benzodioxol-5-yl)-1-(((N-butyl-N-ethylamino)carbonyl)methyl)pyrrolidine-3-carboxylic acid,
trans,trans-2-(4-methoxyphenyl)-4-(1,3-benzodioxol-5-yl)-1-(((N-(2,2-dimethylpropyl)-N-methylamino)carbonyl)methyl)pyrrolidine-3-carboxylic acid,
trans,trans-2-(4-methoxyphenyl)-4-(1,3-benzodioxol-5-yl)-1-(2-(N-((butyl)sulfonyl)-N-methylamino)ethyl)pyrrolidine-3-carboxylic acid,
trans,trans-2-(4-methoxyphenyl)-4-(1,3-benzodioxol-5-yl)-1-(2-(N-methyl-N-((propyl)sulfonyl)amino)ethyl)pyrrolidine-3-carboxylic acid,
(2R,3R,4R)-2-(4-methoxyphenyl)-4-(1,3-benzodioxol-5-yl)-1-(((1R)-1-(N,N-dipropylamino)carbonyl)but-1-yl)pyrrolidine-3-carboxylic acid,
(2S,3S,4S)-2-(4-methoxyphenyl)-4-(1,3-benzodioxol-5-yl)-1-(((1R)-1-(N,N-dipropylamino)carbonyl)but-1-yl)pyrrolidine-3-carboxylic acid,
(2S,3S,4S)-2-(4-methoxyphenyl)-4-(1,3-benzodioxol-5-yl)-1-(((1S)-1-(N,N-dipropylamino)carbonyl)but-1-yl)pyrrolidine-3-carboxylic acid,
(2R,3R,4R)-2-(4-methoxyphenyl)-4-(1,3-benzodioxol-5-yl)-1-(((1S)-1-(N,N-dipropylamino)carbonyl)but-1-yl)pyrrolidine-3-carboxylic acid,
trans,trans-2-(4-methoxyphenyl)-4-(1,3-benzodioxol-5-yl)-1-(((N-butyl-N-propylamino)carbonyl)methyl)pyrrolidine-3-carboxylic acid,
trans,trans-2-(4-methoxyphenyl)-4-(1,3-benzodioxol-5-yl)-1-(2-(N,N-dibutylamino)carbonyl)ethyl)pyrrolidine-3-carboxylic acid,
trans,trans-2-(4-methoxyphenyl)-4-(1,3-benzodioxol-5-yl)-1-(((R,S)-2-(N,N-dibutylamino)carbonyl)ethyl)pyrrolidine-3-carboxylic acid,
trans,trans-2-pentyl-4-(1,3-benzodioxol-5-yl)-1-((N,N-dibutylamino)carbonyl)methyl)pyrrolidine-3-carboxylic acid,
trans,trans-2-pentyl-4-(1,3-benzodioxol-5-yl)-1-(2-(N-propyl-N-((propyl)sulfonyl)amino)ethyl)pyrrolidine-3-carboxylic acid,
trans,trans-2-propyl-4-(1,3-benzodioxol-5-yl)-1-(((N,N-dibutylamino)carbonyl)methyl)pyrrolidine-3-carboxylic acid,
(2R,3R,4S)-2-(4-methoxyphenyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(2-(N-butyl-N-((propyl)sulfonyl)amino)ethyl)pyrrolidine-3-carboxylic acid,
trans,trans-2-(4-methoxyphenyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(2-(N-propyl-N-((propyl)sulfonyl)amino)ethyl)pyrrolidine-3-carboxylic acid,
trans,trans-2-(4-methoxyphenyl)-4-(1,3-benzodioxol-5-yl)-1-(2-(N-butyl-N-((butyl)sulfonyl)amino)ethyl)pyrrolidine-3-carboxylic acid,
trans,trans-2-(4-hydroxyphenyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(2-(N-(2-methylpropyl)-N-((propyl)sulfonyl)amino)ethyl)pyrrolidine-3-carboxylic acid,
trans,trans-2-(4-methoxyphenyl)-4-(1,3-benzodioxol-5-yl)-1-(2-(N-((ethyl)sulfonyl)-N-propylamino)ethyl)pyrrolidine-3-carboxylic acid,
trans,trans-2-(4-methoxyphenyl)-4-(1,3-benzodioxol-5-yl)-1-(2-(N-((butyl)sulfonyl)-N-propylamino)ethyl)pyrrolidine-3-carboxylic acid,
trans,trans-2-(isopropyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(2-(N-((pentyl)sulfonyl)-N-propylamino)ethyl)pyrrolidine-3-carboxylic acid,
trans,trans-2-(4-methoxyphenyl)-4-(1,3-benzodioxol-5-yl)-1-(2-((N-(2-methylpropyl)sulfonyl)-N-propylamino)ethyl)pyrrolidine-3-carboxylic acid,
trans,trans-2-(4-methoxyphenyl)-4-(1,3-benzodioxol-5-yl)-1-(2-(N-((heptyl)sulfonyl)-N-propylamino)ethyl)pyrrolidine-3-carboxylic acid,
trans,trans-2-(4-methoxyphenyl)-4-(1,3-benzodioxol-5-yl)-1-(2-(N-((hexyl)sulfonyl)-N-propylamino)ethyl)pyrrolidine-3-carboxylic acid,
trans,trans-2-(4-methoxyphenyl)-4-(1,3-benzodioxol-5-yl)-1-(2-(N-((pentyl)sulfonyl)-N-ethylamino)ethyl)pyrrolidine-3-carboxylic acid,
trans,trans-2-(3-fluoro-4-methoxyphenyl)-4-(1,3-benzodioxol-5-yl)-1-(((N,N-dibutylamino)carbonyl)methyl)pyrrolidine-3-carboxylic acid,
trans,trans-2-propyl-4-(1,3-benzodioxol-5-yl)-1-(2-(N-((pentyl)sulfonyl)-N-propylamino)ethyl)pyrrolidine-3-carboxylic acid,
trans,trans-2-(3-fluoro-4-methoxyphenyl)-4-(1,3-benzodioxol-5-yl)-1-(2-(N-((pentyl)sulfonyl)-N-propylamino)ethyl)pyrrolidine-3-carboxylic acid,
trans,trans-2-(3-fluoro-4-methoxyphenyl)-4-(1,3-benzodioxol-5-yl)-1-(2-(N-((butyl)sulfonyl)-N-propylamino)ethyl)pyrrolidine-3-carboxylic acid,
trans,trans-2-(4-methoxyphenyl)-4-(1,3-benzodioxol-5-yl)-1-(3-(N-((pentyl)sulfonyl)-N-propylamino)propyl)pyrrolidine-3-carboxylic acid,
trans,trans-2-butyl-4-(1,3-benzodioxol-5-yl)-1-(((N,N-dibutylamino)carbonyl)methyl)pyrrolidine-3-carboxylic acid,
trans,trans-2-(2-methylbutyl)-4-(1,3-benzodioxol-5-yl)-1-(((N,N-dibutylamino)carbonyl)methyl)pyrrolidine-3-carboxylic acid,
trans,trans-2-(3-methylbutyl)-4-(1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(1,3-benzodioxol-5-yl)-1-(2-(N-((hexyl)sulfonyl)-N-propylamino)ethyl)pyrrolidine-3-carboxylic acid,
trans,trans-2-hexyl-4-(1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(7-methoxy-1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(7-methoxy-1,3-benzodioxol-5-yl)-1-(((N-butyl-N-propylamino)carbonyl)methyl)pyrrolidine-3-carboxylic acid,
trans,trans-2-heptyl-4-(1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(1,3-benzodioxol-5-yl)-1-(2-(N-((3-methylbutyl)sulfonyl)-N-propylamino)ethyl)pyrrolidine-3-carboxylic acid,
trans,trans-2-(3-fluoro-4-methoxyphenyl)-4-(7-methoxy-1,3-benzodioxol-5-yl)-1-(2-(N-((pentyl)sulfonyl)-N-propylamino)ethyl)pyrrolidine-3-carboxylic acid,
trans,trans-2-(3-fluoro-4-methoxyphenyl)-4-(1,3-benzodioxol-5-yl)-1-(2-(N-(2-methylpropyl)-N-((pentyl)sulfonyl)amino)ethyl)pyrrolidine-3-carboxylic acid,
trans,trans-2-(4-methoxyphenyl)-4-(1,3-benzodioxol-5-yl)-1-((N-(non-5-ylamino)carbonyl)methyl)pyrrolidine-3-carboxylic acid,
trans,trans-2-(3-fluoro-4-methoxyphenyl)-4-(1,3-benzodioxol-5-yl)-1-(2-(N-((2-methylpropyl)sulfonyl)-N-propylamino)ethyl)pyrrolidine-3-carboxylic acid,
trans,trans-2-(4-methoxyphenyl)-4-(1,3-benzodioxol-5-yl)-1-(((N,N-dihexylamino)carbonyl)methyl)pyrrolidine-3-carboxylic acid,
trans,trans-2-(4-methoxyphenyl)-4-(1,3-benzodioxol-5-yl)-1-(((N-butyl-N-(hept-4-yl)amino)carbonyl)methyl)pyrrolidine-3-carboxylic acid,
trans,trans-2-(2-propylpentyl)-4-(1,3-benzodioxol-5-yl)-1-(((N,N-dibutylamino)carbonyl)methyl)pyrrolidine-3-carboxylic acid,
trans,trans-2-(3-methylpentyl)-4-(1,3-benzodioxol-5-yl)-1-(((N,N-dibutylamino)carbonyl)methyl)pyrrolidine-3-carboxylic acid,
trans,trans-2-(2-ethylbutyl)-4-(1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(1,3-benzodioxol-5-yl)-1-(2-(N-((butyl)sulfonyl)-N-(2-methyl propyl)amino)ethyl)pyrrolidine-3-carboxylic acid,
trans,trans-2-(3-methyl-(E)-pent-3-en-1-yl)-4-(1,3-benzodioxol-5-yl)-1-(((N,N-dibutylamino)carbonyl)methyl)pyrrolidine-3-carboxylic acid,
trans,trans-2-(2-methylpentyl)-4-(1,3-benzodioxol-5-yl)-1-(((N,N-dibutylamino)carbonyl)methyl)pyrrolidine-3-carboxylic acid,
trans,trans-2-(2,2-dimethylphenyl)-4-(1,3-benzodioxol-5-yl)-1-(((N,N-dibutylamino)carbonyl)methyl)pyrrolidine-3-carboxylic acid,
trans,trans-2-(2,2,4-trimethyl-3-(E)-pent-3-enyl)-4-(1,3-benzodioxol-5-yl)-1-(((N,N-dibutylamino)carbonyl)methyl)pyrrolidine-3-carboxylic acid,
trans,trans-2-(2,2,-dimethylpentyl)-4-(7-methoxy-1,3-benzodioxol-5-yl)-1-(((N,N-dibutylamino)carbonyl)methyl)pyrrolidine-3-carboxylic acid,
trans,trans-2-(2,2-dimethyl-(E)-pent-3-enyl)-4-(7-methoxy-1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(((N-(4-aminobutyl)-N-butylamino)carbonyl)methyl)pyrrolidine-3-carboxylic acid,
trans,trans-2-(4-methoxyphenyl)-4-(1,3-benzodioxol-5-yl)-1-(((N-butyl-N-(4-(dimethylamino)butyl)amino)carbonyl)methyl)pyrrolidine-3-carboxylic acid,
(2R,3R,4S)-2-(3-fluoro-4-methoxyphenyl)-4-(1,3-benzodioxol-5-yl)-1-(2-(N-((pentyl)sulfonyl)-N-propylamino)ethyl)pyrrolidine-3-carboxylic acid,
(2S,3R,4S)-2-(2,2-dimethylpentyl)-4-(7-methoxy-1,3-benzodioxol-5-yl)-1-(((N,N-dibutylamino)carbonyl)methyl)pyrrolidine-3-carboxylic acid,
(2S,3R,4S)-2-(2,2-dimethylpentyl)-4-(1,3-benzodioxol-5-yl)-1-(((N,N-dibutylamino)carbonyl)methyl)pyrrolidine-3-carboxylic acid,
(2S,3R,4S)-2-(4-methoxyphenyl)-4-(1,3-benzodioxol-5-yl)-1-(((N-butyl-N-(4-dimethylamino)butyl)amino)carbonyl)methyl)pyrrolidine-3-carboxylic acid,
(2S,3R,4S)-2-(2,2-dimethyl-(E)-pent-3-enyl)-4-(1,3-benzodioxol-5-yl)-1-(((N,N-dibutylamino)carbonyl)methyl)pyrrolidine-3-carboxylic acid, or
(2S,3R,4S)-2-(2,2-dimethyl-(E)-pent-3-enyl)-4-(7-methoxy-1,3-benzodioxol-5-yl)-1-(((N,N-dibutylamino)carbonyl)methyl)pyrrolidine-3-carboxylic acid, or a therapeutically acceptable salt, prodrug, or salt of a prodrug thereof.

68. The method of claim 67 in which the endothelin A receptor antagonist is
(2R,3R,4S)-2-(4-methoxyphenyl)-4-(1,3-benzodioxol-5-yl)-1-(((N,N-dibutylamino)carbonyl)methyl)pyrrolidine-3-carboxylic acid,
(2R,3R,4S)-2-(3-fluoro-4-methoxyphenyl)-4-(1,3-benzodioxol-5-yl)-1-(2-(N-((pentyl)sulfonyl)-N-propylamino)ethyl)pyrrolidine-3-carboxylic acid,
(2S,3R,4S)-2-(2,2-dimethylpentyl)-4-(7-methoxy-1,3-benzodioxol-5-yl)-1-(((N,N-dibutylamino)carbonyl)methyl)pyrrolidine-3-carboxylic acid,
(2S,3R,4S)-2-(2,2-dimethylpentyl)-4-(1,3-benzodioxol-5-yl)-1-(((N,N-dibutylamino)carbonyl)methyl)pyrrolidine-3-carboxylic acid,
(2S,3R,4S)-2-(4-methoxyphenyl)-4-(1,3-benzodioxol-5-yl)-1-(((N-butyl-N-(4-(dimethylamino)butyl)amino)carbonyl)methyl)pyrrolidine-3-carboxylic acid,
(2S,3R,4S)-2-(2,2-dimethyl-(E)-pent-3-enyl)-4-(1,3-benzodioxol-5-yl)-1-(((N,N-dibutylamino)carbonyl)methyl)pyrrolidine-3-carboxylic acid, or
(2S,3R,4S)-2-(2,2-dimethyl-(E)-pent-3-enyl)-4-(7-methoxy-1,3-benzodioxol-5-yl)-1-(((N,N-dibutylamino)carbonyl)methyl)pyrrolidine-3-carboxylic acid, or a therapeutically acceptable salt, prodrug, or salt of a prodrug thereof.

69. The method of claim 68 in which the endothelin A receptor antagonist is
(2R,3R,4S)-2-(4-methoxyphenyl)-4-(1,3-benzodioxol-5-yl)-1-(((N,N-dibutylamino)carbonyl)methyl)pyrrolidine-3-carboxylic acid, or a therapeutically acceptable salt, prodrug, or salt of a prodrug thereof.

70. A method for improving the quality-adjusted time-to-progression of disease in a patient with prostate cancer comprising administering thereto a therapeutically effective amount of an endothelin receptor antagonist.

71. The method of claim 70 in which the health-related quality of life comprises domains of physical functioning, emotional functioning, social/family functioning, role functioning, cognitive functioning, self-perception, and other domains relating to patients with prostate cancer, the other domains comprising pain, fatigue, nausea and vomiting, change in appetite, dyspnea, sleep disturbance, diarrhea, constipation, urinary function, and change in weight, the domains being assessed by the patient.

72. The method of claim 70 in which the endothelin receptor antagonist is administered at or near the beginning of prostate cancer progression.

73. The method of claim 70 in which the endothelin receptor antagonist is administered toward the end of prostate cancer progression.

74. The method of claim 70 in which the therapeutically effective amount of the endothelin receptor antagonist is between about 1 mg per day to about 25 mg per day.

75. The method of claim 74 in which the endothelin receptor antagonist is administered once or twice per day without missing a day.

76. The method of claim 70 in which the endothelin receptor antagonist is an endothelin A receptor antagonist.

77. The method of claim 76 in which the endothelin A receptor antagonist is a compound having formula (I)-a 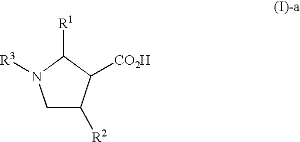
or a compound having formula (I)-a with the relative or absolute stereochemistry shown in the compound having formula (I)-b 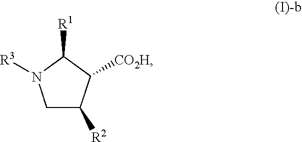
or a therapeutically acceptable salt, prodrug, or salt of prodrug of either, in which
R¹ and R² are independently alkyl, alkenyl, alkynyl, cycloalkyl, aryl, heteroaryl, or alkyl substituted with one cycloalkyl, halo, aryl, heteroaryl, heterocyclyl, —OH, or —O(alkyl) substituent;
R³ is R⁴SO₂R⁴— or R⁴C(O)R⁵—;
R⁴ is alkyl, —(CH₂)alkenyl, —(CH₂)alkynyl, —NR⁶R⁷, alkyl independently substituted with one or two cycloalkyl, aryl, heteroaryl, heterocyclyl, halo, —OH, —O(alkyl), —NH₂, —NH(alkyl), or —N(alkyl)₂ substituents, or alkenyl independently substituted with one or two cycloalkyl, aryl, heteroaryl, heterocyclyl, halo, —OH, —O(alkyl), —NH₂, —NH(alkyl), or —N(alkyl)₂ substituents;
R⁵ is a covalent bond, alkylene, —N(H)(alkylene)-, or —N(alkyl)(alkylene)-, the latter two of which are drawn from left or right, and
R⁶ and R⁷ are independently hydrogen, alkyl, —(CH₂)alkenyl, —(CH₂)alkynyl, cycloalkyl, aryl, or alkyl independently substituted with one or two cycloalkyl, aryl, heteroaryl, heterocyclyl, halo, —OH, —O(alkyl), —OCH₂CF₃, —OCH₂CF₂CF₃, —NH₂, —NH(alkyl), or —N(alkyl)₂ substituents.

78. The method of claim 77 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 2,2-dimethylpentyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

79. The method of claim 77 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 3-fluoro-4-methoxyphenyl; R² is 1,3-benzodioxol-5-yl; and R³is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

80. The method of claim 77 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 4-methoxyphenyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

81. The method of claim 77 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 2,2-dimethylpentyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

82. The method of claim 77 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 3-fluoro-4-methoxyphenyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

83. The method of claim 77 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 4-methoxyphenyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

84. The method of claim 77 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 2,2-dimethylpentyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

85. The method of claim 77 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 3-fluoro-4-methoxyphenyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

86. The method of claim 77 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 4-methoxyphenyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

87. The method of claim 77 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 2,2-dimethylpentyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

88. The method of claim 77 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 3-fluoro-4-methoxyphenyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

89. The method of claim 77 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 4-methoxyphenyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

90. The method of claim 77 in which the endothelin A receptor antagonist is
trans,trans-2-(4-methoxyphenyl)-4-(1,3-benzodioxol-5-yl)-1-(((N-propylamino)carbonyl)methyl)pyrrolidine-3-carboxylic acid,
trans,trans-2-(4-methoxyphenyl)-4-(1,3-benzodioxol-5-yl)-1-(aminocarbonyl)methyl)pyrrolidine-3-carboxylic acid,
trans,trans-2-(4-methoxyphenyl)-4-(1,3-benzodioxol-5-yl)-1-(((N-propenyl)carbonyl)methyl)pyrrolidine-3-carboxylic acid,
trans,trans-2-(4-methoxyphenyl)-4-(1,3-benzodioxol-5-yl)-1-(((N-butylamino)carbonyl)methyl)pyrrolidine-3-carboxylic acid,
trans,trans-2-(4-methoxyphenyl)-4-(1,3-benzodioxol-5-yl)-1-(((N-methyl-N-propylamino)carbonyl)methyl)pyrrolidine-3-carboxylic acid,
trans,trans-2-(4-methoxyphenyl)-4-(1,3-benzodioxol-5-yl)-1-(((N-2-methylpropyl)carbonyl)methyl)pyrrolidine-3-carboxylic acid,
trans,trans-2-(4-methoxyphenyl)-4-(1,4-benzodioxol-6-yl)-1-(((N-propylamino)carbonyl)methyl)pyrrolidine-3-carboxylic acid,
trans,trans-2-(4-methoxyphenyl)-4-(l,4-benzodioxol-6-yl)-1-(((N-methyl-N-propylamino)carbonyl)methyl)pyrrolidine-3-carboxylic acid,
trans,trans-2-(4-methoxyphenyl)-4-(1,3-benzodioxol-5-yl)-1-(((N-butyl-N-methylamino)carbonyl)methyl)pyrrolidine-3-carboxylic acid,
trans,trans-2-(4-methoxyphenyl)-4-(1,3-benzodioxol-5-yl)-1-(((N,N-bis(3-methylbutyl)amino)carbonyl)methyl)pyrrolidine-3-carboxylic acid,
trans,trans-2-(4-methoxyphenyl)-4-(1,3-benzodioxol-5-yl)-1-(((N,N-dipentylamino)carbonyl)methyl)pyrrolidine-3-carboxylic acid,
trans,trans-2-(4-methoxyphenyl)-4-(1,3-benzodioxol-5-yl)-1-(((N-methyl-N-pentylamino)carbonyl)methyl)pyrrolidine-3-carboxylic acid,
trans,trans-2-(4-methoxyphenyl)-4-(1,3-benzodioxol-5-yl)-1-(((N,N-diisobutylamino)carbonyl)methyl)pyrrolidine-3-carboxylic acid,
trans,trans-2-(4-methoxyphenyl)-4-(1,3-benzodioxol-5-yl)-1-(((N-hexyl-N-methylamino)carbonyl)methyl)pyrrolidine-3-carboxylic acid,
trans,trans-2-(4-methoxyphenyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(((N,N-diethylamino)carbonyl)methyl)pyrrolidine-3-carboxylic acid,
trans,trans-2-(4-methoxyphenyl)-4-(1,3-benzodioxol-5-yl)-1-(((N,N-dipropylamino)carbonyl)methyl)pyrrolidine-3-carboxylic acid,
trans,trans-2-(4-methoxyphenyl)-4-(1,3-benzodioxol-5-yl)-1-(((N-isobutyl-N-methylamino)carbonyl)methyl)pyrrolidine-3-carboxylic acid,
trans,trans-2-(4-methoxyphenyl)-4-(1,3-benzodioxol-5-yl)-1-(2-(N-((isopropyl)sulfonyl)amino)ethyl)pyrrolidine-3-carboxylic acid,
trans,trans-2-(4-methoxyphenyl)-4-(1,3-benzodioxol-5-yl)-1-(((N-butyl-N-ethylamino)carbonyl)methyl)pyrrolidine-3-carboxylic acid,
trans,trans-2-(4-methoxyphenyl)-4-(1,3-benzodioxol-5-yl)-1-(((N-(2,2-dimethylpropyl)-N-methylamino)carbonyl)methyl)pyrrolidine-3-carboxylic acid,
trans,trans-2-(4-methoxyphenyl)-4-(1,3-benzodioxol-5-yl)-1-(2-(N-((butyl)sulfonyl)-N-methylamino)ethyl)pyrrolidine-3-carboxylic acid,
trans,trans-2-(4-methoxyphenyl)-4-(1,3-benzodioxol-5-yl)-1-(2-(N-methyl-N-((propyl)sulfonyl)amino)ethyl)pyrrolidine-3-carboxylic acid,
(2R,3R,4R)-2-(4-methoxyphenyl)-4-(1,3-benzodioxol-5-yl)-1-(((1R)-1-(N,N-dipropylamino)carbonyl)but-1-yl)pyrrolidine-3-carboxylic acid,
(2S,3S,4S)-2-(4-methoxyphenyl)-4-(1,3-benzodioxol-5-yl)-1-(((1R)-1-(N,N-dipropylamino)carbonyl)but-1-yl)pyrrolidine-3-carboxylic acid,
(2S,3S,4S)-2-(4-methoxyphenyl)-4-(1,3-benzodioxol-5-yl)-1-(((1S)-1-(N,N-dipropylamino)carbonyl)but-1-yl)pyrrolidine-3-carboxylic acid,
(2R,3R,4R)-2-(4-methoxyphenyl)-4-(1,3-benzodioxol-5-yl)-1-(((1S)-1-(N,N-dipropylamino)carbonyl)but-1-yl)pyrrolidine-3-carboxylic acid,
trans,trans-2-(4-methoxyphenyl)-4-(1,3-benzodioxol-5-yl)-1-(((N-butyl-N-propylamino)carbonyl)methyl)pyrrolidine-3-carboxylic acid,
trans,trans-2-(4-methoxyphenyl)-4-(1,3-benzodioxol-5-yl)-1-(2-(N,N-dibutylamino)carbonyl)ethyl)pyrrolidine-3-carboxylic acid,
trans,trans-2-(4-methoxyphenyl)-4-(1,3-benzodioxol-5-yl)-1-(((R,S)-2-(N,N-dibutylamino)carbonyl)ethyl)pyrrolidine-3-carboxylic acid,
trans,trans-2-pentyl-4-(1,3-benzodioxol-5-yl)-1-((N,N-dibutylamino)carbonyl)methyl)pyrrolidine-3-carboxylic acid,
trans,trans-2-pentyl-4-(1,3-benzodioxol-5-yl)-1-(2-(N-propyl-N-((propyl)sulfonyl)amino)ethyl)pyrrolidine-3-carboxylic acid,
trans,trans-2-propyl-4-(1,3-benzodioxol-5-yl)-1-(((N,N-dibutylamino)carbonyl)methyl)pyrrolidine-3-carboxylic acid,
(2R,3R,4S)-2-(4-methoxyphenyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(2-(N-butyl-N-((propyl)sulfonyl)amino)ethyl)pyrrolidine-3-carboxylic acid,
trans,trans-2-(4-methoxyphenyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(2-(N-propyl-N-((propyl)sulfonyl)amino)ethyl)pyrrolidine-3-carboxylic acid,
trans,trans-2-(4-methoxyphenyl)-4-(1,3-benzodioxol-5-yl)-1-(2-(N-butyl-N-((butyl)sulfonyl)amino)ethyl)pyrrolidine-3-carboxylic acid,
trans,trans-2-(4-hydroxyphenyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(2-(N-(2-methylpropyl)-N-((propyl)sulfonyl)amino)ethyl)pyrrolidine-3-carboxylic acid,
trans,trans-2-(4-methoxyphenyl)-4-(1,3-benzodioxol-5-yl)-1-(2-(N-((ethyl)sulfonyl)-N-propylamino)ethyl)pyrrolidine-3-carboxylic acid,
trans,trans-2-(4-methoxyphenyl)-4-(1,3-benzodioxol-5-yl)-1-(2-(N-((butyl)sulfonyl)-N-propylamino)ethyl)pyrrolidine-3-carboxylic acid,
trans,trans-2-(isopropyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(2-(N-((pentyl)sulfonyl)-N-propylamino)ethyl)pyrrolidine-3-carboxylic acid,
trans,trans-2-(4-methoxyphenyl)-4-(1,3-benzodioxol-5-yl)-1-(2-((N-(2-methylpropyl)sulfonyl)-N-propylamino)ethyl)pyrrolidine-3-carboxylic acid,
trans,trans-2-(4-methoxyphenyl)-4-(1,3-benzodioxol-5-yl)-1-(2-(N-((heptyl)sulfonyl)-N-propylamino)ethyl)pyrrolidine-3-carboxylic acid,
trans,trans-2-(4-methoxyphenyl)-4-(1,3-benzodioxol-5-yl)-1-(2-(N-((hexyl)sulfonyl)-N-propylamino)ethyl)pyrrolidine-3-carboxylic acid,
trans,trans-2-(4-methoxyphenyl)-4-(1,3-benzodioxol-5-yl)-1-(2-(N-((pentyl)sulfonyl)-N-ethylamino)ethyl)pyrrolidine-3-carboxylic acid,
trans,trans-2-(3-fluoro-4-methoxyphenyl)-4-(1,3-benzodioxol-5-yl)-1-(((N,N-dibutylamino)carbonyl)methyl)pyrrolidine-3-carboxylic acid,
trans,trans-2-propyl-4-(1,3-benzodioxol-5-yl)-1-(2-(N-((pentyl)sulfonyl)-N-propylamino)ethyl)pyrrolidine-3-carboxylic acid,
trans,trans-2-(3-fluoro-4-methoxyphenyl)-4-(1,3-benzodioxol-5-yl)-1-(2-(N-((pentyl)sulfonyl)-N-propylamino)ethyl)pyrrolidine-3-carboxylic acid,
trans,trans-2-(3-fluoro-4-methoxyphenyl)-4-(1,3-benzodioxol-5-yl)-1-(2-(N-((butyl)sulfonyl)-N-propylamino)ethyl)pyrrolidine-3-carboxylic acid,
trans,trans-2-(4-methoxyphenyl)-4-(1,3-benzodioxol-5-yl)-1-(3-(N-((pentyl)sulfonyl)-N-propylamino)propyl)pyrrolidine-3-carboxylic acid,
trans,trans-2-butyl-4-(1,3-benzodioxol-5-yl)-1-(((N,N-dibutylamino)carbonyl)methyl)pyrrolidine-3-carboxylic acid,
trans,trans-2-(2-methylbutyl)-4-(1,3-benzodioxol-5-yl)-1-(((N,N-dibutylamino)carbonyl)methyl)pyrrolidine-3-carboxylic acid,
trans,trans-2-(3-methylbutyl)-4-(1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(1,3-benzodioxol-5-yl)-1-(2-(N-((hexyl)sulfonyl)-N-propylamino)ethyl)pyrrolidine-3-carboxylic acid,
trans,trans-2-hexyl-4-(1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(7-methoxy-1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(7-methoxy-1,3-benzodioxol-5-yl)-1-(((N-butyl-N-propylamino)carbonyl)methyl)pyrrolidine-3-carboxylic acid,
trans,trans-2-heptyl-4-(1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(1,3-benzodioxol-5-yl)-1-(2-(N-((3-methylbutyl)sulfonyl)-N-propylamino)ethyl)pyrrolidine-3-carboxylic acid,
trans,trans-2-(3-fluoro-4-methoxyphenyl)-4-(7-methoxy-1,3-benzodioxol-5-yl)-1-(2-(N-((pentyl)sulfonyl)-N-propylamino)ethyl)pyrrolidine-3-carboxylic acid,
trans,trans-2-(3-fluoro-4-methoxyphenyl)-4-(1,3-benzodioxol-5-yl)-1-(2-(N-(2-methylpropyl)-N-((pentyl)sulfonyl)amino)ethyl)pyrrolidine-3-carboxylic acid,
trans,trans-2-(4-methoxyphenyl)-4-(1,3-benzodioxol-5-yl)-1-((N-(non-5-ylamino)carbonyl)methyl)pyrrolidine-3-carboxylic acid,
trans,trans-2-(3-fluoro-4-methoxyphenyl)-4-(1,3-benzodioxol-5-yl)-1-(2-(N-((2-methylpropyl)sulfonyl)-N-propylamino)ethyl)pyrrolidine-3-carboxylic acid,
trans,trans-2-(4-methoxyphenyl)-4-(1,3-benzodioxol-5-yl)-1-(((N,N-dihexylamino)carbonyl)methyl)pyrrolidine-3-carboxylic acid,
trans,trans-2-(4-methoxyphenyl)-4-(1,3-benzodioxol-5-yl)-1-(((N-butyl-N-(hept-4-yl)amino)carbonyl)methyl)pyrrolidine-3-carboxylic acid,
trans,trans-2-(2-propylpentyl)-4-(1,3-benzodioxol-5-yl)-1-(((N,N-dibutylamino)carbonyl)methyl)pyrrolidine-3-carboxylic acid,
trans,trans-2-(3-methylpentyl)-4-(1,3-benzodioxol-5-yl)-1-(((N,N-dibutylamino)carbonyl)methyl)pyrrolidine-3-carboxylic acid,
trans,trans-2-(2-ethylbutyl)-4-(1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(1,3-benzodioxol-5-yl)-1-(2-(N-((butyl)sulfonyl)-N-(2-methylpropyl)amino)ethyl)pyrrolidine-3-carboxylic acid,
trans,trans-2-(3-methyl-(E)-pent-3-en-1-yl)-4-(1,3-benzodioxol-5-yl)-1-(((N,N-dibutylamino)carbonyl)methyl)pyrrolidine-3-carboxylic acid,
trans,trans-2-(2-methylpentyl)-4-(1,3-benzodioxol-5-yl)-1-(((N,N-dibutylamino)carbonyl)methyl)pyrrolidine-3-carboxylic acid,
trans,trans-2-(2,2-dimethylphenyl)-4-(1,3-benzodioxol-5-yl)-1-(((N,N-dibutylamino)carbonyl)methyl)pyrrolidine-3-carboxylic acid,
trans,trans-2-(2,2,4-trimethyl-3-(E)-pent-3-enyl)-4-(1,3-benzodioxol-5-yl)-1-(((N,N-dibutylamino)carbonyl)methyl)pyrrolidine-3-carboxylic acid,
trans,trans-2-(2,2,-dimethylpentyl)-4-(7-methoxy-1,3-benzodioxol-5-yl)-1-(((N,N-dibutylamino)carbonyl)methyl)pyrrolidine-3-carboxylic acid,
trans,trans-2-(2,2-dimethyl-(E)-pent-3-enyl)-4-(7-methoxy-1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(((N-(4-aminobutyl)-N-butylamino)carbonyl)methyl)pyrrolidine-3-carboxylic acid,
trans,trans-2-(4-methoxyphenyl)-4-(1,3-benzodioxol-5-yl)-1-(((N-butyl-N-(4-(dimethylamino)butyl)amino)carbonyl)methyl)pyrrolidine-3-carboxylic acid,
(2R,3R,4S)-2-(3-fluoro-4-methoxyphenyl)-4-(1,3-benzodioxol-5-yl)-1-(2-(N-((pentyl)sulfonyl)-N-propylamino)ethyl)pyrrolidine-3-carboxylic acid,
(2S,3R,4S)-2-(2,2-dimethylpentyl)-4-(7-methoxy-1,3-benzodioxol-5-yl)-1-(((N,N-dibutylamino)carbonyl)methyl)pyrrolidine-3-carboxylic acid,
(2S,3R,4S)-2-(2,2-dimethylpentyl)-4-(1,3-benzodioxol-5-yl)-1-(((N,N-dibutylamino)carbonyl)methyl)pyrrolidine-3-carboxylic acid,
(2S,3R,4S)-2-(4-methoxyphenyl)-4-(1,3-benzodioxol-5-yl)-1-(((N-butyl-N-(4-(dimethylamino)butyl)amino)carbonyl)methyl)pyrrolidine-3-carboxylic acid,
(2S,3R,4S)-2-(2,2-dimethyl-(E)-pent-3-enyl)-4-(1,3-benzodioxol-5-yl)-1-(((N,N-dibutylamino)carbonyl)methyl)pyrrolidine-3-carboxylic acid, or
(2S,3R,4S)-2-(2,2-dimethyl-(E)-pent-3-enyl)-4-(7-methoxy-1,3-benzodioxol-5-yl)-1-(((N,N-dibutylamino)carbonyl)methyl)pyrrolidine-3-carboxylic acid, or a therapeutically acceptable salt, prodrug, or salt of a prodrug thereof.

91. The method of claim 90 in which the endothelin A receptor antagonist is
(2R,3R,4S)-2-(4-methoxyphenyl)-4-(1,3-benzodioxol-5-yl)-1-(((N,N-dibutylamino)carbonyl)methyl)pyrrolidine-3-carboxylic acid,
(2R,3R,4S)-2-(3-fluoro-4-methoxyphenyl)-4-(1,3-benzodioxol-5-yl)-1-(2-(N-((pentyl)sulfonyl)-N-propylamino)ethyl)pyrrolidine-3-carboxylic acid,
(2S,3R,4S)-2-(2,2-dimethylpentyl)-4-(7-methoxy-1,3-benzodioxol-5-yl)-1-(((N,N-dibutylamino)carbonyl)methyl)pyrrolidine-3-carboxylic acid,
(2S,3R,4S)-2-(2,2-dimethylpentyl)-4-(1,3-benzodioxol-5-yl)-1-(((N,N-dibutylamino)carbonyl)methyl)pyrrolidine-3-carboxylic acid,
(2S,3R,4S)-2-(4-methoxyphenyl)-4-(1,3-benzodioxol-5-yl)-1-(((N-butyl-N-(4-(dimethylamino)butyl)amino)carbonyl)methyl)pyrrolidine-3-carboxylic acid,
(2S,3R,4S)-2-(2,2-dimethyl-(E)-pent-3-enyl)-4-(1,3-benzodioxol-5-yl)-1-(((N,N-dibutylamino)carbonyl)methyl)pyrrolidine-3-carboxylic acid, or
(2S,3R,4S)-2-(2,2-dimethyl-(E)-pent-3-enyl)-4-(7-methoxy-1,3-benzodioxol-5-yl)-1-(((N,N-dibutylamino)carbonyl)methyl)pyrrolidine-3-carboxylic acid, or a therapeutically acceptable salt, prodrug, or salt of a prodrug thereof.

92. The method of claim 91 in which the endothelin A receptor antagonist is
(2R,3R,4S)-2-(4-methoxyphenyl)-4-(1,3-benzodioxol-5-yl)-1-(((N,N-dibutylamino)carbonyl)methyl)pyrrolidine-3-carboxylic acid, or a therapeutically acceptable salt, prodrug, or salt of a prodrug thereof.

93. A method for extending the quality-adjusted time-to-progression of disease in a patient with prostate cancer comprising administering thereto a therapeutically effective amount of an endothelin receptor antagonist.

94. The method of claim 93 in which the health-related quality of life comprises domains of physical functioning, emotional functioning, social/family functioning, role functioning, cognitive functioning, self-perception, and other domains relating to patients with prostate cancer, the other domains comprising pain, fatigue, nausea and vomiting, change in appetite, dyspnea, sleep disturbance, diarrhea, constipation, urinary function, and change in weight, the domains being assessed by the patient.

95. The method of claim 93 in which the endothelin receptor antagonist is administered at or near the beginning of prostate cancer progression.

96. The method of claim 93 in which the endothelin receptor antagonist is administered toward the end of prostate cancer progression.

97. The method of claim 93 in which the therapeutically effective amount of the endothelin receptor antagonist is between about 1 mg per day to about 25 mg per day.

98. The method of claim 97 in which the endothelin receptor antagonist is administered once or twice per day without missing a day.

99. The method of claim 93 in which the endothelin receptor antagonist is an endothelin A receptor antagonist.

100. The method of claim 99 in which the endothelin A receptor antagonist is a compound having formula (I)-a 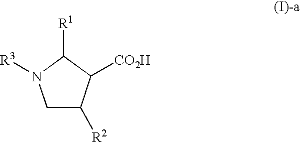
or a compound having formula (I)-a with the relative or absolute stereochemistry shown in the compound having formula (I)-b 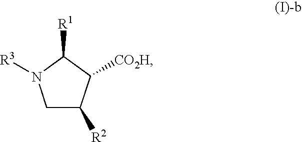
or a therapeutically acceptable salt, prodrug, or salt of prodrug of either, in which
R¹ and R² are independently alkyl, alkenyl, alkynyl, cycloalkyl, aryl, heteroaryl, or alkyl substituted with one cycloalkyl, halo, aryl, heteroaryl, heterocyclyl, —OH, or —O(alkyl) substituent;
R³ is R⁴SO₂R⁵— or R⁴C(O)R⁵—;
R⁴ is alkyl, —(CH₂)alkenyl, —(CH₂)alkynyl, —NR⁶R⁷, alkyl independently substituted with one or two cycloalkyl, aryl, heteroaryl, heterocyclyl, halo, —OH, —O(alkyl), —NH₂, —NH(alkyl), or —N(alkyl)₂ substituents, or alkenyl independently substituted with one or two cycloalkyl, aryl, heteroaryl, heterocyclyl, halo, —OH, —O(alkyl), —NH₂, —NH(alkyl), or —N(alkyl)₂ substituents;
R⁵ is a covalent bond, alkylene, —N(H)(alkylene)-, or —N(alkyl)(alkylene)-, the latter two of which are drawn from left or right, and
R⁶ and R⁷ are independently hydrogen, alkyl, —(CH₂)alkenyl, —(CH₂)alkynyl, cycloalkyl, aryl, or alkyl independently substituted with one or two cycloalkyl, aryl, heteroaryl, heterocyclyl, halo, —OH, —O(alkyl), —OCH₂CF₃, —OCH₂CF₂CF₃, —NH₂, —NH(alkyl), or —N(alkyl)₂ substituents.

101. The method of claim 100 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 2,2-dimethylpentyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

102. The method of claim 100 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 3-fluoro-4-methoxyphenyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

103. The method of claim 100 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 4-methoxyphenyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

104. The method of claim 100 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 2,2-dimethylpentyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

105. The method of claim 100 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 3-fluoro-4-methoxyphenyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

106. The method of claim 100 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 4-methoxyphenyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

107. The method of claim 100 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 2,2-dimethylpentyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

108. The method of claim 100 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 3-fluoro-4-methoxyphenyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

109. The method of claim 100 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 4-methoxyphenyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

110. The method of claim 100 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 2,2-dimethylpentyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

111. The method of claim 100 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 3-fluoro-4-methoxyphenyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

112. The method of claim 100 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 4-methoxyphenyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

113. The method of claim 100 in which the endothelin A receptor antagonist is
trans,trans-2-(4-methoxyphenyl)-4-(1,3-benzodioxol-5-yl)-1-(((N-propylamino)carbonyl)methyl)pyrrolidine-3-carboxylic acid,
trans,trans-2-(4-methoxyphenyl)-4-(1,3-benzodioxol-5-yl)-1-(aminocarbonyl)methyl)pyrrolidine-3-carboxylic acid,
trans,trans-2-(4-methoxyphenyl)-4-(1,3-benzodioxol-5-yl)-1-(((N-propenyl)carbonyl)methyl)pyrrolidine-3-carboxylic acid,
trans,trans-2-(4-methoxyphenyl)-4-(1,3-benzodioxol-5-yl)-1-(((N-butylamino)carbonyl)methyl)pyrrolidine-3-carboxylic acid,
trans,trans-2-(4-methoxyphenyl)-4-(1,3-benzodioxol-5-yl)-1-(((N-methyl-N-propylamino)carbonyl)methyl)pyrrolidine-3-carboxylic acid,
trans,trans-2-(4-methoxyphenyl)-4-(1,3-benzodioxol-5-yl)-1-(((N-2-methylpropyl)carbonyl)methyl)pyrrolidine-3-carboxylic acid,
trans,trans-2-(4-methoxyphenyl)-4-(1,4-benzodioxol-6-yl)-1-(((N-propylamino)carbonyl)methyl)pyrrolidine-3-carboxylic acid,
trans,trans-2-(4-methoxyphenyl)-4-(1,4-benzodioxol-6-yl)-1-(((N-methyl-N-propylamino)carbonyl)methyl)pyrrolidine-3-carboxylic acid,
trans,trans-2-(4-methoxyphenyl)-4-(1,3-benzodioxol-5-yl)-1-((N-butyl-N-methylamino)carbonyl)methyl)pyrrolidine-3-carboxylic acid,
trans,trans-2-(4-methoxyphenyl)-4-(1,3-benzodioxol-5-yl)-1-(((N,N-bis(3-methylbutyl)amino)carbonyl)methyl)pyrrolidine-3-carboxylic acid,
trans,trans-2-(4-methoxyphenyl)-4-(1,3-benzodioxol-5-yl)-1-(((N,N-dipentylamino)carbonyl)methyl)pyrrolidine-3-carboxylic acid,
trans,trans-2-(4-methoxyphenyl)-4-(1,3-benzodioxol-5-yl)-1-(((N-methyl-N-pentylamino)carbonyl)methyl)pyrrolidine-3-carboxylic acid,
trans,trans-2-(4-methoxyphenyl)-4-(1,3-benzodioxol-5-yl)-1-(((N,N-diisobutylamino)carbonyl)methyl)pyrrolidine-3-carboxylic acid,
trans,trans-2-(4-methoxyphenyl)-4-(1,3-benzodioxol-5-yl)-1-(((N-hexyl-N-methylamino)carbonyl)methyl)pyrrolidine-3-carboxylic acid,
trans,trans-2-(4-methoxyphenyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(((N,N-diethylamino)carbonyl)methyl)pyrrolidine-3-carboxylic acid,
trans,trans-2-(4-methoxyphenyl)-4-(1,3-benzodioxol-5-yl)-1-(((N,N-dipropylamino)carbonyl)methyl)pyrrolidine-3-carboxylic acid,
trans,trans-2-(4-methoxyphenyl)-4-(1,3-benzodioxol-5-yl)-1-(((N-isobutyl-N-methylamino)carbonyl)methyl)pyrrolidine-3-carboxylic acid,
trans,trans-2-(4-methoxyphenyl)-4-(1,3-benzodioxol-5-yl)-1-(2-(N-((isopropyl)sulfonyl)amino)ethyl)pyrrolidine-3-carboxylic acid,
trans,trans-2-(4-methoxyphenyl)-4-(1,3-benzodioxol-5-yl)-1-(((N-butyl-N-ethylamino)carbonyl)methyl)pyrrolidine-3-carboxylic acid,
trans,trans-2-(4-methoxyphenyl)-4-(1,3-benzodioxol-5-yl)-1-(((N-(2,2-dimethylpropyl)-N-methylamino)carbonyl)methyl)pyrrolidine-3-carboxylic acid,
trans,trans-2-(4-methoxyphenyl)-4-(1,3-benzodioxol-5-yl)-1-(2-(N-((butyl)sulfonyl)-N-methylamino)ethyl)pyrrolidine-3-carboxylic acid,
trans,trans-2-(4-methoxyphenyl)-4-(1,3-benzodioxol-5-yl)-1-(2-(N-methyl-N-((propyl)sulfonyl)amino)ethyl)pyrrolidine-3-carboxylic acid,
(2R,3R,4R)-2-(4-methoxyphenyl)-4-(1,3-benzodioxol-5-yl)-1-(((1R)-1-(N,N-dipropylamino)carbonyl)but-1-yl)pyrrolidine-3-carboxylic acid,
(2S,3S,4S)-2-(4-methoxyphenyl)-4-(1,3-benzodioxol-5-yl)-1-(((1R)-1-(N,N-dipropylamino)carbonyl)but-1-yl)pyrrolidine-3-carboxylic acid,
(2S,3S,4S)-2-(4-methoxyphenyl)-4-(1,3-benzodioxol-5-yl)-1-(((1S)-1-(N,N-dipropylamino)carbonyl)but-1-yl)pyrrolidine-3-carboxylic acid,
(2R,3R,4R)-2-(4-methoxyphenyl)-4-(1,3-benzodioxol-5-yl)-1-(((1S)-1-(N,N-dipropylamino)carbonyl)but-1-yl)pyrrolidine-3-carboxylic acid,
trans,trans-2-(4-methoxyphenyl)-4-(1,3-benzodioxol-5-yl)-1-(((N-butyl-N-propylamino)carbonyl)methyl)pyrrolidine-3-carboxylic acid,
trans,trans-2-(4-methoxyphenyl)-4-(1,3-benzodioxol-5-yl)-1-(2-(N,N-dibutylamino)carbonyl)ethyl)pyrrolidine-3-carboxylic acid,
trans,trans-2-(4-methoxyphenyl)-4-(1,3-benzodioxol-5-yl)-1-(((R,S)-2-(N,N-dibutylamino)carbonyl)ethyl)pyrrolidine-3-carboxylic acid,
trans,trans-2-pentyl-4-(1,3-benzodioxol-5-yl)-1-((N,N-dibutylamino)carbonyl)methyl)pyrrolidine-3-carboxylic acid,
trans,trans-2-pentyl-4-(1,3-benzodioxol-5-yl)-1-(2-(N-propyl-N-((propyl)sulfonyl)amino)ethyl)pyrrolidine-3-carboxylic acid,
trans,trans-2-propyl-4-(1,3-benzodioxol-5-yl)-1-(((N,N-dibutylamino)carbonyl)methyl)pyrrolidine-3-carboxylic acid,
(2R,3R,4S)-2-(4-methoxyphenyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(2-(N-butyl-N-((propyl)sulfonyl)amino)ethyl)pyrrolidine-3-carboxylic acid,
trans,trans-2-(4-methoxyphenyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(2-(N-propyl-N-((propyl)sulfonyl)amino)ethyl)pyrrolidine-3-carboxylic acid,
trans,trans-2-(4-methoxyphenyl)-4-(1,3-benzodioxol-5-yl)-1-(2-(N-butyl-N-((butyl)sulfonyl)amino)ethyl)pyrrolidine-3-carboxylic acid,
trans,trans-2-(4-hydroxyphenyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(2-(N-(2-methylpropyl)-N-((propyl)sulfonyl)amino)ethyl)pyrrolidine-3-carboxylic acid,
trans,trans-2-(4-methoxyphenyl)-4-(1,3-benzodioxol-5-yl)-1-(2-(N-((ethyl)sulfonyl)-N-propylamino)ethyl)pyrrolidine-3-carboxylic acid,
trans,trans-2-(4-methoxyphenyl)-4-(1,3-benzodioxol-5-yl)-1-(2-(N-((butyl)sulfonyl)-N-propylamino)ethyl)pyrrolidine-3-carboxylic acid,
trans,trans-2-(isopropyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(2-(N-((pentyl)sulfonyl)-N-propylamino)ethyl)pyrrolidine-3-carboxylic acid,
trans,trans-2-(4-methoxyphenyl)-4-(1,3-benzodioxol-5-yl)-1-(2-((N-(2-methylpropyl)sulfonyl)-N-propylamino)ethyl)pyrrolidine-3-carboxylic acid,
trans,trans-2-(4-methoxyphenyl)-4-(1,3-benzodioxol-5-yl)-1 (2-(N-((heptyl)sulfonyl)-N-propylamino)ethyl)pyrrolidine-3-carboxylic acid,
trans,trans-2-(4-methoxyphenyl)-4-(1,3-benzodioxol-5-yl)-1-(2-(N-((hexyl)sulfonyl)-N-propylamino)ethyl)pyrrolidine-3-carboxylic acid,
trans,trans-2-(4-methoxyphenyl)-4-(1,3-benzodioxol-5-yl)-1-(2-(N-((pentyl)sulfonyl)-N-ethylamino)ethyl)pyrrolidine-3-carboxylic acid,
trans,trans-2-(3-fluoro-4-methoxyphenyl)-4-(1,3-benzodioxol-5-yl)-1-(((N,N-dibutylamino)carbonyl)methyl)pyrrolidine-3-carboxylic acid,
trans,trans-2-propyl-4-(1,3-benzodioxol-5-yl)-1-(2(N-((pentyl)sulfonyl)-N-propylamino)ethyl)pyrrolidine-3-carboxylic acid,
trans,trans-2-(3-fluoro-4-methoxyphenyl)-4-(1,3-benzodioxol-5-yl)-1-(2-(N-((pentyl)sulfonyl)-N-propylamino)ethyl)pyrrolidine-3-carboxylic acid,
trans,trans-2-(3-fluoro-4-methoxyphenyl)-4-(1,3-benzodioxol-5-yl)-1-(2-(N-((butyl)sulfonyl)-N-propylamino)ethyl)pyrrolidine-3-carboxylic acid,
trans,trans-2-(4-methoxyphenyl)-4-(1,3-benzodioxol-5- yl)-1-(3-(N-((pentyl)sulfonyl)-N-propylamino)propyl)pyrrolidine-3-carboxylic acid,
trans,trans-2-butyl-4-(1,3-benzodioxol-5-yl)-1-(((N,N-dibutylamino)carbonyl)methyl)pyrrolidine-3-carboxylic acid,
trans,trans-2-(2-methylbutyl)-4-(1,3-benzodioxol-5-yl)-1-(((N,N-dibutylamino)carbonyl)methyl)pyrrolidine-3-carboxylic acid,
trans,trans-2-(3-methylbutyl)-4-(1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(1,3-benzodioxol-5-yl)-1-(2-(N-((hexyl)sulfonyl)-N-propylamino)ethyl)pyrrolidine-3-carboxylic acid,
trans,trans-2-hexyl-4-(1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(7-methoxy-1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(7-methoxy-1,3-benzodioxol-5-yl)-1-(((N-butyl-N-propylamino)carbonyl)methyl)pyrrolidine-3-carboxylic acid,
trans,trans-2-heptyl-4-(1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(1,3-benzodioxol-5-yl)-1-(2-(N-((3-methylbutyl)sulfonyl)-N-propylamino)ethyl)pyrrolidine-3-carboxylic acid,
trans,trans-2-(3-fluoro-4-methoxyphenyl)-4-(7-methoxy-1,3-benzodioxol-5-yl)-1-(2-(N-((pentyl)sulfonyl)-N-propylamino)ethyl)pyrrolidine-3-carboxylic acid,
trans,trans-2-(3-fluoro-4-methoxyphenyl)-4-(1,3-benzodioxol-5-yl)-1-(2-(N-(2-methylpropyl)-N-((pentyl)sulfonyl)amino)ethyl)pyrrolidine-3-carboxylic acid,
trans,trans-2-(4-methoxyphenyl)-4-(1,3-benzodioxol-5-yl)-1-((N-(non-5-ylamino)carbonyl)methyl)pyrrolidine-3-carboxylic acid,
trans,trans-2-(3-fluoro-4-methoxyphenyl)-4-(1,3-benzodioxol-5-yl)-1-(2-(N-((2-methylpropyl)sulfonyl)-N-propylamino)ethyl)pyrrolidine-3-carboxylic acid,
trans,trans-2-(4-methoxyphenyl)-4-(1,3-benzodioxol-5-yl)-1-(((N,N-dihexylamino)carbonyl)methyl)pyrrolidine-3-carboxylic acid,
trans,trans-2-(4-methoxyphenyl)-4-(1,3-benzodioxol-5-yl)-1-(((N-butyl-N-(hept-4-yl)amino)carbonyl)methyl)pyrrolidine-3-carboxylic acid,
trans,trans-2-(2-propylpentyl)-4-(1,3-benzodioxol-5-yl)-1-(((N,N-dibutylamino)carbonyl)methyl)pyrrolidine-3-carboxylic acid,
trans,trans-2-(3-methylpentyl)-4-(1,3-benzodioxol-5-yl)-1-(((N,N-dibutylamino)carbonyl)methyl)pyrrolidine-3-carboxylic acid,
trans,trans-2-(2-ethylbutyl)-4-(1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(1,3-benzodioxol-5-yl)-1-(2-(N-((butyl)sulfonyl)-N-(2-methylpropyl)amino)ethyl)pyrrolidine-3-carboxylic acid,
trans,trans-2-(3-methyl-(E)-pent-3-en-1-yl)-4-(1,33-benzodioxol-5-yl)-1-(((N,N-dibutylamino)carbonyl)methyl)pyrrolidine-3-carboxylic acid,
trans,trans-2-(2-methylpentyl)-4-(1,3-benzodioxol-5-yl)-1-(((N,N-dibutylamino)carbonyl)methyl)pyrrolidine-3-carboxylic acid,
trans,trans-2-(2,2-dimethylphenyl)-4-(1,3-benzodioxol-5-yl)-1-(((N,N-dibutylamino)carbonyl)methyl)pyrrolidine-3-carboxylic acid,
trans,trans-2-(2,2,4-trimethyl-3-(E)-pent-3-enyl)-4-(1,3-benzodioxol-5-yl)-1-(((N,N-dibutylamino)carbonyl)methyl)pyrrolidine-3-carboxylic acid,
trans,trans-2-(2,2,-dimethylpentyl)-4-(7-methoxy-1,3-benzodioxol-5-yl)-1-(((N,N-dibutylamino)carbonyl)methyl)pyrrolidine-3-carboxylic acid,
trans,trans-2-(2,2-dimethyl-(E)-pent-3-enyl)-4-(7-methoxy-1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(((N-(4-aminobutyl)-N-butylamino)carbonyl)methyl)pyrrolidine-3-carboxylic acid,
trans,trans-2-(4-methoxyphenyl)-4-(1,3-benzodioxol-5-yl)-1-(((N-butyl-N-(4-(dimethylamino)butyl)amino)carbonyl)methyl)pyrrolidine-3-carboxylic acid,
(2R,3R,4S)-2-(3-fluoro-4-methoxyphenyl)-4-(1,3-benzodioxol-5-yl)-1-(2-(N-((pentyl)sulfonyl)-N-propylamino)ethyl)pyrrolidine-3-carboxylic acid,
(2S,3R,4S)-2-(2,2-dimethylpentyl)-4-(7-methoxy-1,3-benzodioxol-5-yl)-1-(((N,N-dibutylamino)carbonyl)methyl)pyrrolidine-3-carboxylic acid,
(2S,3R,4S)-2-(2,2-dimethylpentyl)-4-(1,3-benzodioxol-5-yl)-1-(((N,N-dibutylamino)carbonyl)methyl)pyrrolidine-3-carboxylic acid,
(2S,3R,4S)-2-(4-methoxyphenyl)-4-(1,3-benzodioxol-5-yl)-1-(((N-butyl-N-(4-(dimethylamino)butyl)amino)carbonyl)methyl)pyrrolidine-3-carboxylic acid,
(2S,3R,4S)-2-(2,2-dimethyl-(E)-pent-3-enyl)-4-(1,3-benzodioxol-5-yl)-1-(((N,N-dibutylamino)carbonyl)methyl)pyrrolidine-3-carboxylic acid, or
(2S,3R,4S)-2-(2,2-dimethyl-(E)-pent-3-enyl)-4-(7-methoxy-1,3-benzodioxol-5-yl)-1-(((N,N-dibutylamino)carbonyl)methyl)pyrrolidine-3-carboxylic acid, or a therapeutically acceptable salt, prodrug, or salt of a prodrug thereof.

114. The method of claim 113 in which the endothelin A receptor antagonist is
(2R,3R,4S)-2-(4-methoxyphenyl)-4-(1,3-benzodioxol-5-yl)-1-(((N,N-dibutylamino)carbonyl)methyl)pyrrolidine-3-carboxylic acid,
(2R,3R,4S)-2-(3-fluoro-4-methoxyphenyl)-4-(1,3-benzodioxol-5-yl)-1-(2-(N-((pentyl)sulfonyl)-N-propylamino)ethyl)pyrrolidine-3-carboxylic acid,
(2S,3R,4S)-2-(2,2-dimethylpentyl)-4-(7-methoxy-1,3-benzodioxol-5-yl)-1-(((N,N-dibutylamino)carbonyl)methyl)pyrrolidine-3-carboxylic acid,
(2S,3R,4S)-2-(2,2-dimethylpentyl)-4-(1,3-benzodioxol-5-yl)-1-(((N,N-dibutylamino)carbonyl)methyl)pyrrolidine-3-carboxylic acid,
(2S,3R,4S)-2-(4-methoxyphenyl)-4-(1,3-benzodioxol-5-yl)-1-(((N-butyl-N-(4-(dimethylamino)butyl)amino)carbonyl)methyl)pyrrolidine-3-carboxylic acid,
(2S,3R,4S)-2-(2,2-dimethyl-(E)-pent-3-enyl)-4-(1,3-benzodioxol-5-yl)-1-(((N,N-dibutylamino)carbonyl)methyl)pyrrolidine-3-carboxylic acid, or
(2S,3R,4S)-2-(2,2-dimethyl-(E)-pent-3-enyl)-4-(7-methoxy-1,3-benzodioxol-5-yl)-1-(((N,N-dibutylamino)carbonyl)methyl)pyrrolidine-3-carboxylic acid, or a therapeutically acceptable salt, prodrug, or salt of a prodrug thereof.

115. The method of claim 114 in which the endothelin A receptor antagonist is
(2R,3R,4S)-2-(4-methoxyphenyl)-4-(1,3-benzodioxol-5-yl)-1-(((N,N-dibutylamino)carbonyl)methyl)pyrrolidine-3-carboxylic acid, or a therapeutically acceptable salt, prodrug, or salt of a prodrug thereof.

116. A method for determining modulation of the health-related quality-adjusted time-to-progression of disease in a patient undergoing endothelin antagonist chemotherapy for prostate cancer, the method comprising the steps of:
(a) providing a patient population;
(b) administering to each member of the patient population either a therapeutically effective amount of an ET receptor antagonist or placebo;
(c) measuring the health-related QoL domains of each patient over a period of time to provide a health-related QATTP of disease for each patient in the patient population; and
(d) determining the health-related QATTP for each health-related QoL domain and the sum of the mean or median health-related QATTP's of disease for the patient population.

117. The method of claim 116 in which the patient population comprises about 280 patients with prostate cancer.

118. The method of claim 116 in which the endothelin receptor antagonist sustains the health-related quality-adjusted time-to-progression of disease in the patient with prostate cancer.

119. The method of claim 116 in which the endothelin receptor antagonist extends the health-related quality-adjusted time-to-progression of disease the a patient with prostate cancer.

120. The method of claim 116 in which the endothelin receptor antagonist improves the health-related quality-adjusted time-to-progression of disease in a patient with prostate cancer.

121. The method of claim 116 in which the endothelin receptor antagonist is administered at or near the beginning of prostate cancer progression.

122. The method of claim 116 in which the endothelin receptor antagonist is administered toward the end of prostate cancer progression.

123. The method of claim 116 in which the health-related quality of life domains comprise physical functioning, emotional functioning, social/family functioning, role functioning, cognitive functioning, self-perception, and other domains relating to patients with prostate cancer, the other domains comprising pain, fatigue, nausea and vomiting, change in appetite, dyspnea, sleep disturbance, diarrhea, constipation, urinary function, and change in weight, the domains being assessed by the patient.

124. The method of claim 116 in which the period of time is about six weeks after the beginning of the treatment.

125. The method of claim 116 in which the therapeutically effective amount of the endothelin receptor antagonist is between about 1 mg per day to about 25 mg per day.

126. The method of claim 125 in which the endothelin receptor antagonist is administered once or twice per day without missing a day.

127. The method of claim 116 in which the endothelin receptor antagonist is an endothelin A receptor antagonist.

128. The method of claim 127 in which the endothelin A receptor antagonist is a compound having formula (I)-a 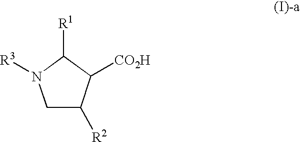
or a compound having formula (I)-a with the relative or absolute stereochemistry shown in the compound having formula (I)-b 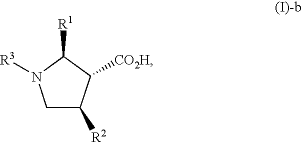
or a therapeutically acceptable salt, prodrug, or salt of prodrug of either, in which
R¹ and R² are independently alkyl, alkenyl, alkynyl, cycloalkyl, aryl, heteroaryl, or alkyl substituted with one cycloalkyl, halo, aryl, heteroaryl, heterocyclyl, —OH, or —O(alkyl) substituent;
R³ is R⁴SO₂R⁵— or R⁴C(O)R⁵—;
R⁴ is alkyl, —(CH₂)alkenyl, —(CH₂)alkynyl, —NR⁶R⁷, alkyl independently substituted with one or two cycloalkyl, aryl, heteroaryl, heterocyclyl, halo, —OH, —O(alkyl), —NH₂, —NH(alkyl), or —N(alkyl)₂ substituents, or alkenyl independently substituted with one or two cycloalkyl, aryl, heteroaryl, heterocyclyl, halo, —OH, —O(alkyl), —NH₂, —NH(alkyl), or —N(alkyl)₂ substituents;
R⁵ is a covalent bond, alkylene, —N(H)(alkylene)-, or —N(alkyl)(alkylene)-, the latter two of which are drawn from left or right, and
R⁶ and R⁷ are independently hydrogen, alkyl, —(CH₂)alkenyl, —(CH₂)alkynyl, cycloalkyl, aryl, or alkyl independently substituted with one or two cycloalkyl, aryl, heteroaryl, heterocyclyl, halo, —OH, —O(alkyl), —OCH₂CF₃, —OCH₂CF₂CF₃, —NH₂, —NH(alkyl), or —N(alkyl)₂ substituents.

129. The method of claim 128 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 2,2-dimethylpentyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

130. The method of claim 128 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 3-fluoro-4-methoxyphenyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

131. The method of claim 128 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 4-methoxyphenyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

132. The method of claim 128 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 2,2-dimethylpentyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

133. The method of claim 128 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 3-fluoro-4-methoxyphenyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

134. The method of claim 128 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 4-methoxyphenyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

135. The method of claim 128 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 2,2-dimethylpentyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

136. The method of claim 128 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 3-fluoro-4-methoxyphenyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

137. The method of claim 128 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 4-methoxyphenyl; R² is 1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

138. The method of claim 128 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 2,2-dimethylpentyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

139. The method of claim 128 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 3-fluoro-4-methoxyphenyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

140. The method of claim 128 in which the endothelin A receptor antagonist is a compound having formula (I)-a with the relative stereochemistry shown in the compound having formula (I)-b, or a therapeutically acceptable salt, prodrug, or salt of a prodrug thereof, in which R¹ is 4-methoxyphenyl; R² is 7-methoxy-1,3-benzodioxol-5-yl; and R³ is ((N,N-dibutylamino)carbonyl)methyl, ((N-butyl-N-(4-(dimethylamino)butyl)amino)carbonyl)methyl, or 2-(N-((pentyl)sulfonyl)-N-propylamino)ethyl.

141. The method of claim 128 in which the endothelin A receptor antagonist is
trans,trans-2-(4-methoxyphenyl)-4-(1,3-benzodioxol-5-yl)-1-(((N -propylamino)carbonyl)methyl)pyrrolidine-3-carboxylic acid,
trans,trans-2-(4-methoxyphenyl)-4-(1,3-benzodioxol-5-yl)-1-(aminocarbonyl)methyl)pyrrolidine-3-carboxylic acid,
trans,trans-2-(4-methoxyphenyl)-4-(1,3-benzodioxol-5-yl)-1-(((N-propenyl)carbonyl)methyl)pyrrolidine-3-carboxylic acid,
trans,trans-2-(4-methoxyphenyl)-4-(1,3-benzodioxol-5-yl)-1-(((N-butylamino)carbonyl)methyl)pyrrolidine-3-carboxylic acid,
trans,trans-2-(4-methoxyphenyl)-4-(1,3-benzodioxol-5-yl)-1-(((N-methyl-N-propylamino)carbonyl)methyl)pyrrolidine-3-carboxylic acid,
trans,trans-2-(4-methoxyphenyl)-4-(1,3-benzodioxol-5-yl)-1-(((N-2-methylpropyl)carbonyl)methyl)pyrrolidine-3-carboxylic acid,
trans,trans-2-(4-methoxyphenyl)-4-(1,4-benzodioxol-6-yl)-1-(((N-propylamino)carbonyl)methyl)pyrrolidine-3-carboxylic acid,
trans,trans-2-(4-methoxyphenyl)-4-(1,4-benzodioxol-6-yl)-1-(((N-methyl-N-propylamino)carbonyl)methyl)pyrrolidine-3-carboxylic acid,
trans,trans-2-(4-methoxyphenyl)-4-(1,3-benzodioxol-5-yl)-1-(((N-butyl-N-methylamino)carbonyl)methyl)pyrrolidine-3-carboxylic acid,
trans,trans-2-(4-methoxyphenyl)-4-(1,3-benzodioxol-5-yl)-1-(((N,N-bis(3-methylbutyl)amino)carbonyl)methyl)pyrrolidine-3-carboxylic acid,
trans,trans-2-(4-methoxyphenyl)-4-(1,3-benzodioxol-5-yl)-1-(((N,N-dipentylamino)carbonyl)methyl)pyrrolidine-3-carboxylic acid,
trans,trans-2-(4-methoxyphenyl)-4-(1,3-benzodioxol-5-yl)-1-(((N-methyl-N-pentylamino)carbonyl)methyl)pyrrolidine-3-carboxylic acid,
trans,trans-2-(4-methoxyphenyl)-4-(1,3-benzodioxol-5-yl)-1-(((N,N-diisobutylamino)carbonyl)methyl)pyrrolidine-3-carboxylic acid,
trans,trans-2-(4-methoxyphenyl)-4-(1,3-benzodioxol-5-yl)-1-(((N-hexyl-N-methylamino)carbonyl)methyl)pyrrolidine-3-carboxylic acid,
trans,trans-2-(4-methoxyphenyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(((N,N-diethylamino)carbonyl)methyl)pyrrolidine-3-carboxylic acid,
trans,trans-2-(4-methoxyphenyl)-4-(1,3-benzodioxol-5-yl)-1-(((N,N-dipropylamino)carbonyl)methyl)pyrrolidine-3-carboxylic acid,
trans,trans-2-(4-methoxyphenyl)-4-(1,3-benzodioxol-5-yl)-1-(((N-isobutyl-N-methylamino)carbonyl)methyl)pyrrolidine-3-carboxylic acid,
trans,trans-2-(4-methoxyphenyl)-4-(1,3-benzodioxol-5-yl)-1-(2-(N-((isopropyl)sulfonyl)amino)ethyl)pyrrolidine-3-carboxylic acid,
trans,trans-2-(4-methoxyphenyl)-4-(1,3-benzodioxol-5-yl)-1-(((N-butyl-N-ethylamino)carbonyl)methyl)pyrrolidine-3-carboxylic acid,
trans,trans-2-(4-methoxyphenyl)-4-(1,3-benzodioxol-5-yl)-1-(((N-(2,2-dimethylpropyl)-N-methylamino)carbonyl)methyl)pyrrolidine-3-carboxylic acid,
trans,trans-2-(4-methoxyphenyl)-4-(1,3-benzodioxol-5-yl)-1-(2-(N-((butyl)sulfonyl)-N-methylamino)ethyl)pyrrolidine-3-carboxylic acid,
trans,trans-2-(4-methoxyphenyl)-4-(1,3-benzodioxol-5-yl)-1-(2-(N-methyl-N-((propyl)sulfonyl)amino)ethyl)pyrrolidine-3-carboxylic acid,
(2R,3R,4R)-2-(4-methoxyphenyl)-4-(1,3-benzodioxol-5-yl)-1-(((1R)-1-(N,N-dipropylamino)carbonyl)but-1-yl)pyrrolidine-3-carboxylic acid,
(2S,3S,4S)-2-(4-methoxyphenyl)-4-(1,3-benzodioxol-5-yl)-1-(((1R)-1-(N,N-dipropylamino)carbonyl)but-1-yl)pyrrolidine-3-carboxylic acid,
(2S,3S,4S)-2-(4-methoxyphenyl)-4-(1,3-benzodioxol-5-yl)-1-(((1S)-1-(N,N-dipropylamino)carbonyl)but-1-yl)pyrrolidine-3-carboxylic acid,
(2R,3R,4R)-2-(4-methoxyphenyl)-4-(1,3-benzodioxol-5-yl)-1-(((1S)-1-(N,N-dipropylamino)carbonyl)but-1-yl)pyrrolidine-3-carboxylic acid,
trans,trans-2-(4-methoxyphenyl)-4-(1,3-benzodioxol-5-yl)-1-(((N-butyl-N-propylamino)carbonyl)methyl)pyrrolidine-3-carboxylic acid,
trans,trans-2-(4-methoxyphenyl)-4-(1,3-benzodioxol-5-yl)-1-(2-(N,N-dibutylamino)carbonyl)ethyl)pyrrolidine-3-carboxylic acid,
trans,trans-2-(4-methoxyphenyl)-4-(1,3-benzodioxol-5-yl)-1-(((R,S)-2-(N,N-dibutylamino)carbonyl)ethyl)pyrrolidine-3-carboxylic acid,
trans,trans-2-pentyl-4-(1,3-benzodioxol-5-yl)-1-((N,N-dibutylamino)carbonyl)methyl)pyrrolidine-3-carboxylic acid,
trans,trans-2-pentyl-4-(1,3-benzodioxol-5-yl)-1-(2-(N-propyl-N-((propyl)sulfonyl)amino)ethyl)pyrrolidine-3-carboxylic acid,
trans,trans-2-propyl-4-(1,3-benzodioxol-5-yl)-1-(((N,N-dibutylamino)carbonyl)methyl)pyrrolidine-3-carboxylic acid,
(2R,3R,4S)-2-(4-methoxyphenyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(2-(N-butyl-N-((propyl)sulfonyl)amino)ethyl)pyrrolidine-3-carboxylic acid,
trans,trans-2-(4-methoxyphenyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(2-(N-propyl-N-((propyl)sulfonyl)amino)ethyl)pyrrolidine-3-carboxylic acid,
trans,trans-2-(4-methoxyphenyl)-4-(1,3-benzodioxol-5-yl)-1-(2-(N-butyl-N-((butyl)sulfonyl)amino)ethyl)pyrrolidine-3-carboxylic acid,
trans,trans-2-(4-hydroxyphenyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(2-(N-(2-methylpropyl)-N-((propyl)sulfonyl)amino)ethyl)pyrrolidine-3-carboxylic acid,
trans,trans-2-(4-methoxyphenyl)-4-(1,3-benzodioxol-5-yl)-1-(2-(N-((ethyl)sulfonyl)-N-propylamino)ethyl)pyrrolidine-3-carboxylic acid,
trans,trans-2-(4-methoxyphenyl)-4-(1,3-benzodioxol-5-yl)-1-(2-(N-((butyl)sulfonyl)-N-propylamino)ethyl)pyrrolidine-3-carboxylic acid,
trans,trans-2-(isopropyl)-4-(1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(2-(N-((pentyl)sulfonyl)-N-propylamino)ethyl)pyrrolidine-3-carboxylic acid,
trans,trans-2-(4-methoxyphenyl)-4-(1,3-benzodioxol-5-yl)-1-(2-((N-(2-methylpropyl)sulfonyl)-N-propylamino)ethyl)pyrrolidine-3-carboxylic acid,
trans,trans-2-(4-methoxyphenyl)-4-(1,3-benzodioxol-5-yl)-1-(2-(N-((heptyl)sulfonyl)-N-propylamino)ethyl)pyrrolidine-3-carboxylic acid,
trans,trans-2-(4-methoxyphenyl)-4-(1,3-benzodioxol-5-yl)-1-(2-(N-((hexyl)sulfonyl)-N-propylamino)ethyl)pyrrolidine-3-carboxylic acid,
trans,trans-2-(4-methoxyphenyl)-4-(1,3-benzodioxol-5-yl)-1-(2-(N-((pentyl)sulfonyl)-N-ethylamino)ethyl)pyrrolidine-3-carboxylic acid,
trans,trans-2-(3-fluoro-4-methoxyphenyl)-4-(1,3-benzodioxol-5-yl)-1-(((N,N-dibutylamino)carbonyl)methyl)pyrrolidine-3-carboxylic acid,
trans,trans-2-propyl-4-(1,3-benzodioxol-5-yl)-1-(2-(N-((pentyl)sulfonyl)-N-propylamino)ethyl)pyrrolidine-3-carboxylic acid,
trans,trans-2-(3-fluoro-4-methoxyphenyl)-4-(1,3-benzodioxol-5-yl)-1-(2-(N-((pentyl)sulfonyl)-N-propylamino)ethyl)pyrrolidine-3-carboxylic acid,
trans,trans-2-(3-fluoro-4-methoxyphenyl)-4-(1,3-benzodioxol-5-yl)-1-(2-(N-((butyl)sulfonyl)-N-propylamino)ethyl)pyrrolidine-3-carboxylic acid,
trans,trans-2-(4-methoxyphenyl)-4-(1,3-benzodioxol-5-yl)-1-(3-(N-((pentyl)sulfonyl)-N-propylamino)propyl)pyrrolidine-3-carboxylic acid,
trans,trans-2-butyl-4-(1,3-benzodioxol-5-yl)-1-(((N,N-dibutylamino)carbonyl)methyl)pyrrolidine-3-carboxylic acid,
trans,trans-2-(2-methylbutyl)-4-(1,3-benzodioxol-5-yl)-1-(((N,N-dibutylamino)carbonyl)methyl)pyrrolidine-3-carboxylic acid,
trans,trans-2-(3-methylbutyl)-4-(1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(1,3-benzodioxol-5-yl)-1-(2-(N-((hexyl)sulfonyl)-N-propylamino)ethyl)pyrrolidine-3-carboxylic acid,
trans,trans-2-hexyl-4-(1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(7-methoxy-1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(7-methoxy-1,3-benzodioxol-5-yl)-1-(((N-butyl-N-propylamino)carbonyl)methyl)pyrrolidine-3-carboxylic acid,
trans,trans-2-heptyl-4-(1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(1,3-benzodioxol-5-yl)-1-(2-(N-((3-methylbutyl)sulfonyl)-N-propylamino)ethyl)pyrrolidine-3-carboxylic acid,
trans,trans-2-(3-fluoro-4-methoxyphenyl)-4-(7-methoxy-1,3-benzodioxol-5-yl)-1-(2-(N-((pentyl)sulfonyl)-N-propylamino)ethyl)pyrrolidine-3-carboxylic acid,
trans,trans-2-(3-fluoro-4-methoxyphenyl)-4-(1,3-benzodioxol-5-yl)-1-(2-(N-(2-methylpropyl)-N-((pentyl)sulfonyl)amino)ethyl)pyrrolidine-3-carboxylic acid,
trans,trans-2-(4-methoxyphenyl)-4-(1,3-benzodioxol-5-yl)-1-((N-(non-5-ylamino)carbonyl)methyl)pyrrolidine-3-carboxylic acid,
trans,trans-2-(3-fluoro-4-methoxyphenyl)-4-(1,3-benzodioxol-5-yl)-1-(2-(N-((2-methylpropyl)sulfonyl)-N-propylamino)ethyl)pyrrolidine-3-carboxylic acid,
trans,trans-2-(4-methoxyphenyl)-4-(1,3-benzodioxol-5-yl)-1-(((N,N-dihexylamino)carbonyl)methyl)pyrrolidine-3-carboxylic acid,
trans,trans-2-(4-methoxyphenyl)-4-(1,3-benzodioxol-5-yl)-1-(((N-butyl-N-(hept-4-yl)amino)carbonyl)methyl)pyrrolidine-3-carboxylic acid,
trans,trans-2-(2-propylpentyl)-4-(1,3-benzodioxol-5-yl)-1-(((N,N-dibutylamino)carbonyl)methyl)pyrrolidine-3-carboxylic acid,
trans,trans-2-(3-methylpentyl)-4-(1,3-benzodioxol-5-yl)-1-(((N,N-dibutylamino)carbonyl)methyl)pyrrolidine-3-carboxylic acid,
trans,trans-2-(2-ethylbutyl)-4-(1,3-benzodioxol-5-yl)-1-(((N,N-dibutylamino)carbonyl)methyl)pyrrolidine-3-carboxylic acid,
trans,trans-2-(3-fluoro-4-methoxyphenyl)-4-(1,3-benzodioxol-5-yl)-1-(2-(N-((butyl)sulfonyl)-N-(2-methylpropyl)amino)ethyl)pyrrolidine-3-carboxylic acid,
trans,trans-2-(3-methyl-(E)-pent-3-en-1-yl)-4-(1,3-benzodioxol-5-yl)-1-(((N,N-dibutylamino)carbonyl)methyl)pyrrolidine-3-carboxylic acid,
trans,trans-2-(2-methylpentyl)-4-(1,3-benzodioxol-5-yl)-1-(((N,N-dibutylamino)carbonyl)methyl)pyrrolidine-3-carboxylic acid,
trans,trans-2-(2,2-dimethylphenyl)-4-(1,3-benzodioxol-5-yl)-1-(((N,N-dibutylamino)carbonyl)methyl)pyrrolidine-3-carboxylic acid,
trans,trans-2-(2,2,4-trimethyl-3-(E)-pent-3-enyl)-4-(1,3-benzodioxol-5-yl)-1-(((N,N-dibutylamino)carbonyl)methyl)pyrrolidine-3-carboxylic acid,
trans,trans-2-(2,2,-dimethylpentyl)-4-(7-methoxy-1,3-benzodioxol-5-yl)-1-(((N,N-dibutylamino)carbonyl)methyl)pyrrolidine-3-carboxylic acid,
trans,trans-2-(2,2-dimethyl-(E)-pent-3-enyl)-4-(7-methoxy-1,3-benzodioxol-5-yl)-1-(((N,N-dibutylamino)carbonyl)methyl)pyrrolidine-3-carboxylic acid,
trans,trans-2-(4-methoxyphenyl)-4-(1,3-benzodioxol-5-yl)-1-(((N-(4-aminobutyl)-N-butylamino)carbonyl)methyl)pyrrolidine-3-carboxylic acid,
trans,trans-2-(4-methoxyphenyl)-4-(1,3-benzodioxol-5-yl)-1-(((N-butyl-N-(4-(dimethylamino)butyl)amino)carbonyl)methyl)pyrrolidine-3-carboxylic acid,
(2R,3R,4S)-2-(3-fluoro-4-methoxyphenyl)-4-(1,3-benzodioxol-5-yl)-1-(2-(N-((pentyl)sulfonyl)-N-propylamino)ethyl)pyrrolidine-3-carboxylic acid,
(2S,3R,4S)-2-(2,2-dimethylpentyl)-4-(7-methoxy-1,3-benzodioxol-5-yl)-1-(((N,N-dibutylamino)carbonyl)methyl)pyrrolidine-3-carboxylic acid,
(2S,3R,4S)-2-(2,2-dimethylpentyl)-4-(1,3-benzodioxol-5-yl)-1-(((N,N-dibutylamino)carbonyl)methyl)pyrrolidine-3-carboxylic acid,
(2S,3R,4S)-2-(4-methoxyphenyl)-4-(1,3-benzodioxol-5-yl)-1-(((N-butyl-N-(4-(dimethylamino)butyl)amino)carbonyl)methyl)pyrrolidine-3-carboxylic acid,
(2S,3R,4S)-2-(2,2-dimethyl-(E)-pent-3-enyl)-4-(1,3-benzodioxol-5-yl)-1-(((N,N-dibutylamino)carbonyl)methyl)pyrrolidine-3-carboxylic acid, or
(2S,3R,4S)-2-(2,2-dimethyl-(E)-pent-3-enyl)-4-(7-methoxy-1,3-benzodioxol-5-yl)-1-(((N,N-dibutylamino)carbonyl)methyl)pyrrolidine-3-carboxylic acid, or a therapeutically acceptable salt, prodrug, or salt of a prodrug thereof.

142. The method of claim 141 in which the endothelin A receptor antagonist is
(2R,3R,4S)-2-(4-methoxyphenyl)-4-(1,3-benzodioxol-5-yl)-1-(((N,N-dibutylamino)carbonyl)methyl)pyrrolidine-3-carboxylic acid,
(2R,3R,4S)-2-(3-fluoro-4-methoxyphenyl)-4-(1,3-benzodioxol-5-yl)-1-(2-(N-((pentyl)sulfonyl)-N-propylamino)ethyl)pyrrolidine-3-carboxylic acid,
(2S,3R,4S)-2-(2,2-dimethylpentyl)-4-(7-methoxy-1,3-benzodioxol-5-yl)-1-(((N,N-dibutylamino)carbonyl)methyl)pyrrolidine-3-carboxylic acid,
(2S,3R,4S)-2-(2,2-dimethylpentyl)-4-(1,3-benzodioxol-5-yl)-1-(((N,N-dibutylamino)carbonyl)methyl)pyrrolidine-3-carboxylic acid,
(2S,3R,4S)-2-(4-methoxyphenyl)-4-(1,3-benzodioxol-5-yl)-1-(((N-butyl-N-(4-(dimethylamino)butyl)amino)carbonyl)methyl)pyrrolidine-3-carboxylic acid,
(2S,3R,4S)-2-(2,2-dimethyl-(E)-pent-3-enyl)-4-(1,3-benzodioxol-5-yl)-1-(((N,N-dibutylamino)carbonyl)methyl)pyrrolidine-3-carboxylic acid, or
(2S,3R,4S)-2-(2,2-dimethyl-(E)-pent-3-enyl)-4-(7-methoxy-1,3-benzodioxol-5-yl)-1-(((N,N-dibutylamino)carbonyl)methyl)pyrrolidine-3-carboxylic acid, or a therapeutically acceptable salt, prodrug, or salt of a prodrug thereof.

143. The method of claim 142 in which the endothelin A receptor antagonist is
(2R,3R,4S)-2-(4-methoxyphenyl)-4-(1,3-benzodioxol-5-yl)-1-(((N,N-dibutylamino)carbonyl)methyl)pyrrolidine-3-carboxylic acid, or a therapeutically acceptable salt, prodrug, or salt of a prodrug thereof.
